(19) United States
(12) Patent Application Publication
(10) Pub. No.: US 2024/0052009 A1
(43) Pub. Date: Feb. 15, 2024

(54) STABILIZED TCR CONSTRUCTS AND METHODS OF USE

(71) Applicant: ZYMEWORKS BC INC., Vancouver (CA)

(72) Inventors: Eric ESCOBAR-CABRERA, Vancouver, BC (CA); Lee FREIBURGER, Vancouver, BC (CA); Daniel T. PATTON, Vancouver, BC (CA); Leonard G. PRESTA, Vancouver, BC (CA); Patrick FARBER, Vancouver, BC (CA); Charles Michael STEVENS, Vancouver, BC (CA)

(21) Appl. No.: 18/258,057

(22) PCT Filed: Dec. 21, 2021
(86) PCT No.: PCT/CA2021/051855

§ 371 Date: Jun. 16, 2023

(51) Int. Cl. C07K 14/725; C07K 16/28

(52) U.S. Cl. CPC C07K 14/7051; C07K 16/2809; C07K 2319/30; A61K 38/00

(57) ABSTRACT

Stabilized TCR constructs comprising a TCR alpha chain polypeptide having a variable alpha (Vα) domain and a constant alpha (Cα) domain and a TCR beta chain polypeptide having a variable beta (Vβ) domain and a constant beta (Cβ) domain. The TCR constructs are stabilized by the introduction into the Cα domain and/or the Cβ domain of stabilizing mutations such as non-naturally occurring disulfide bonds between the Cα domain and the Cβ domain (an interchain disulfide bond), non-naturally occurring intrachain disulfide bonds, point mutations, loop truncation mutations, and combinations thereof. TCR fusion proteins comprising one or more of the TCR constructs and a scaffold and/or other biologically active moiety are also described.

### FIELD

The present disclosure relates to the field of soluble T-cell receptors for use as therapeutics and, in particular, to stabilized TCR constructs and TCR fusion proteins.

### BACKGROUND

T-cell receptors (TCRs) are proteins found on the surface of T-cells. TCRs modulate the immune response through binding with Class I and Class II major histocompatibility complexes (MHC) present on the surface of cells. An MHC presenting a peptide sequence which activates a T-cell via the TCR triggers an immune response. In the case of cancer, a mutated or overexpressed peptide sequence can be presented on the surface of the cancerous cell. TCRs can differentiate between peptides with a single amino acid mutation and thus provide an opportunity to specifically target these mutant peptide-MHC complexes.

TCRs belong to the immunoglobulin super-family (IgSF) of proteins and share certain structural similarities with antibodies. Similar to the Fab section of an antibody, a TCR includes two unique chains, each containing one variable domain and one constant domain, with highly variable loops (CDRs) in the variable domain providing the binding selectivity of the TCR.

TCRs are membrane-bound proteins that contain a transmembrane domain. There has been interest in developing a soluble form of TCRs as therapeutics, but soluble TCRs are inherently unstable proteins with low expression and stability. Modifications to improve the stability of soluble TCRs have been described. International Patent Publication No. WO 2004/074322 describes a stabilized soluble TCR that comprises a disulfide bond between constant domain residues which is not present in the native TCR. International Patent Publication No. WO 2016/070814 describes a high-stability soluble TCR comprising an artificial interchain disulfide bond linking the constant domains of the TCRα and β chains, and International Patent Publication No. WO 2016/184258 describes a stabilized soluble heterodimeric TCR containing an artificial interchain disulfide bond between the variable region of the α chain and the constant region of the β chain. Point mutations that improve stability of soluble TCRs have also been described (see, Shusta, et al., 2000, Nature Biotechnol., 18:754-759, and Gunnarsen, et al., 2013, Scientific Reports, 3:1162).

Fusion of the extracellular portion of a TCR to different domains of human immunoglobulins (Ig) has also been used as a strategy to increase the expression and/or improve stability of the TCR (see, Lunde, et al., 2010, BMC Biotechnol., 10:61; Ozawa, et al., 2012, Biochem. Biophys. Res. Commun., 422:245-249; and Wu, et al., 2015, MAbs, 7:364-376).

International Patent Publication No. WO 1999/018129 describes a single-chain TCR (sc-TCR) in which the alpha and beta chain of the TCR are connected with a flexible linker. The sc-TCR was shown to have improved stability. sc-TCR fusion proteins are also described which include covalently linked TCR Vα and Vβ chains fused to an immunoglobin light chain constant region. U.S. Pat. Nos. 6,534,633 and 8,105,830 describe an sc-TCR covalently linked through a peptide linker sequence to at least one single-chain antibody (sc-Ab).

This background information is provided for the purpose of making known information believed by the applicant to be of possible relevance to the present disclosure. No admission is necessarily intended, nor should be construed, that any of the preceding information constitutes prior art against the claimed invention.

### SUMMARY

Described herein are stabilized TCR constructs and methods of use. In one aspect, the disclosure relates to a TCR construct comprising a TCR alpha chain polypeptide and a TCR beta chain polypeptide, the TCR alpha chain polypeptide comprising a variable alpha (Vα) domain and a constant alpha (Cα) domain and the TCR beta chain polypeptide comprising a variable beta (Vβ) domain and a constant beta (Cβ) domain, where the Cα domain and Cβ domain comprise stabilizing mutations, the stabilizing mutations comprising a first interchain disulfide bond between the Cα domain and the Cβ domain and one or more additional stabilizing mutations, the one or more additional stabilizing mutations selected from: a) an interchain disulfide bond formed between: i) a cysteine residue comprised by an amino acid extension at the C-terminus of the Cβ domain of the TCR beta chain polypeptide, wherein the amino acid extension is 1 to about 10 amino acids in length, and ii) a cysteine residue at position TRAC 128 in the Cα domain of the TCR alpha chain polypeptide; b) an interchain disulfide bond between cysteine residue substitutions at positions TRAC 84.2 and TRBC 79; c) an interchain disulfide bond between cysteine residue substitutions at positions TRAC 122 and TRBC 12; d) an interchain disulfide bond between cysteine residue substitutions at positions TRAC 124 and TRBC 11; e) an interchain disulfide bond between cysteine residue substitutions at positions TRAC 125 and TRBC 11; f) an interchain disulfide bond between cysteine residue substitutions at positions TRAC 126 and TRBC 11; g) an interchain disulfide bond between cysteine residue substitutions at positions TRAC 127 and TRBC 11; h) an interchain disulfide bond between cysteine residue substitutions at positions TRAC 128 and TRBC 11; i) an intrachain disulfide bond between cysteine residue substitutions at positions TRAC 39 and TRAC 85; j) an intrachain disulfide bond between cysteine residue substitutions at positions TRAC 26 and TRAC 85.1; k) an amino acid substitution at position TRAC 4 from Val to Ala, Thr, Ile, Leu or Met; l) an amino acid substitution at position TRAC 26 from Thr to Ala, Val, Ile, Leu or Met; m) an amino acid substitution at position TRAC 39 from Val to Ala, Thr, Ile, Leu or Met; n) an amino acid substitution at position TRAC 85 from Ala to Ser, Thr, Val, Ile or Met; o) an amino acid substitution at position TRAC 105 from Ala to Ser, Thr, Glu, Gln, Asp, Asn, His, Lys or Arg; p) an amino acid substitution at position TRAC 120 from Phe to Tyr or His; q) an amino acid substitution at position TRBC 6 from Val to Ala, Thr, Ile, Leu or Met; r) an amino acid substitution at position TRBC 36 from His to Phe, Tyr or Trp; s) an amino acid substitution at position TRBC 86 from Ser to Ala or Thr; t) an amino acid substitution at position TRBC 45.3 from Val to Ser, Thr, Glu, Gln, Asp, Asn, His, Lys or Arg; u) a deletion of 1 to 4 consecutive amino acids of the DE loop in the Cβ domain of the TCR construct, and v) a replacement of the amino acids at positions TRBC 84.4 to 85.4 with an amino acid sequence of 2 to 4 amino acids, wherein the amino acid sequence allows for formation of a beta-turn, where the numbering of amino acids is IMGT numbering, and where the TCR construct has an increased TCR melting temperature (Tm) as compared to a corresponding TCR construct comprising the first non-naturally occurring disulfide bond alone.

In another aspect, the present disclosure relates to a TCR construct comprising a TCR alpha chain polypeptide and a TCR beta chain polypeptide, the TCR alpha chain polypeptide comprising a variable alpha (Vα) domain and a constant alpha (Cα) domain and the TCR beta chain polypeptide comprising a variable beta (Vβ) domain and a constant beta (Cβ) domain, where the Cα domain and/or Cβ domain comprise one or more stabilizing mutations selected from: a) an interchain disulfide bond formed between: i) a cysteine residue comprised by an amino acid extension at the C-terminus of the Cβ domain of the TCR beta chain polypeptide, wherein the amino acid extension is 1 to about 10 amino acids in length, and ii) a cysteine residue at position TRAC 128 in the Cα domain of the TCR alpha chain polypeptide; b) an interchain disulfide bond between cysteine residue substitutions at positions TRAC 84.2 and TRBC 79; c) an interchain disulfide bond between cysteine residue substitutions at positions TRAC 124 and TRBC 11; d) an interchain disulfide bond between cysteine residue substitutions at positions TRAC 125 and TRBC 11; e) an interchain disulfide bond between cysteine residue substitutions at positions TRAC 126 and TRBC 11; f) an interchain disulfide bond between cysteine residue substitutions at positions TRAC 127 and TRBC 11; g) an interchain disulfide bond between cysteine residue substitutions at positions TRAC 128 and TRBC 11; h) an intrachain disulfide bond between cysteine residue substitutions at positions TRAC 39 and TRAC 85; i) an intrachain disulfide bond between cysteine residue substitutions at positions TRAC 26 and TRAC 85.1; j) an amino acid substitution at position TRAC 4 from Val to Ala, Thr, Ile, Leu or Met; k) an amino acid substitution at position TRAC 26 from Thr to Ala, Val, Ile, Leu or Met; l) an amino acid substitution at position TRAC 39 from Val to Ala, Thr, Ile, Leu or Met; m) an amino acid substitution at position TRAC 85 from Ala to Ser, Thr, Val, Ile or Met; n) an amino acid substitution at position TRAC 105 from Ala to Ser, Thr, Glu, Gln, Asp, Asn, His, Lys or Arg; o) an amino acid substitution at position TRAC 120 from Phe to Tyr or His; p) an amino acid substitution at position TRBC 6 from Val to Ala, Thr, Ile, Leu or Met; q) an amino acid substitution at position TRBC 36 from His to Phe, Tyr or Trp; r) an amino acid substitution at position TRBC 86 from Ser to Ala or Thr; s) an amino acid substitution at position TRBC 45.3 from Val to Ser, Thr, Glu, Gln, Asp, Asn, His, Lys or Arg; t) a deletion of 1 to 4 consecutive amino acids of the DE loop in the Cβ domain of the TCR construct, and u) a replacement of the amino acids at positions TRBC 84.4 to 85.4 with an amino acid sequence of 2 to 4 amino acids, wherein the amino acid sequence allows for formation of a beta-turn, where the numbering of amino acids is IMGT numbering, and wherein the TCR construct has an increased TCR melting temperature (Tm) as compared to a corresponding TCR construct that does not comprise the one or more stabilizing mutations.

In another aspect, the present disclosure relates to a TCR construct comprising a TCR alpha chain polypeptide and a TCR beta chain polypeptide, the TCR alpha chain polypeptide comprising a variable alpha (Vα) domain and a constant alpha (Cα) domain and the TCR beta chain polypeptide comprising a variable beta (Vβ) domain and a constant beta (Cβ) domain, where the Cα domain and Cβ domain together comprise two or more stabilizing mutations selected from: a) an interchain disulfide bond formed between: i) a cysteine residue comprised by an amino acid extension at the C-terminus of the Cβ domain of the TCR beta chain polypeptide, wherein the amino acid extension is 1 to about 10 amino acids in length, and ii) a cysteine residue at position TRAC 128 in the Cα domain of the TCR alpha chain polypeptide; b) an interchain disulfide bond between cysteine residue substitutions at positions TRAC 84 and TRBC 79; c) an interchain disulfide bond between cysteine residue substitutions at positions TRAC 84.2 and TRBC 79; d) an interchain disulfide bond between cysteine residue substitutions at positions TRAC 124 and TRBC 11; e) an interchain disulfide bond between cysteine residue substitutions at positions TRAC 122 and TRBC 12; f) an interchain disulfide bond between cysteine residue substitutions at positions TRAC 125 and TRBC 11; g) an interchain disulfide bond between cysteine residue substitutions at positions TRAC 126 and TRBC 11; h) an interchain disulfide bond between cysteine residue substitutions at positions TRAC 127 and TRBC 11; i) an interchain disulfide bond between cysteine residue substitutions at positions TRAC 128 and TRBC 11; j) an intrachain disulfide bond between cysteine residue substitutions at positions TRAC 39 and TRAC 85; k) an intrachain disulfide bond between cysteine residue substitutions at positions TRAC 26 and TRAC 85.1; 1) an amino acid substitution at position TRAC 4 from Val to Ala, Thr, Ile, Leu or Met; m) an amino acid substitution at position TRAC 26 from Thr to Ala, Val, Ile, Leu or Met; n) an amino acid substitution at position TRAC 39 from Val to Ala, Thr, Ile, Leu or Met; o) an amino acid substitution at position TRAC 85 from Ala to Ser, Thr, Val, Ile or Met; p) an amino acid substitution at position TRAC 105 from Ala to Ser, Thr, Glu, Gln, Asp, Asn, His, Lys or Arg; q) an amino acid substitution at position TRAC 120 from Phe to Tyr or His; r) an amino acid substitution at position TRBC 6 from Val to Ala, Thr, Ile, Leu or Met; s) an amino acid substitution at position TRBC 36 from His to Phe, Tyr or Trp; t) an amino acid substitution at position TRBC 86 from Ser to Ala or Thr; u) an amino acid substitution at position TRBC 45.3 from Val to Ser, Thr, Glu, Gln, Asp, Asn, His, Lys or Arg; v) a deletion of 1 to 4 consecutive amino acids of the DE loop in the Cβ domain of the TCR construct, and w) a replacement of the amino acids at positions TRBC 84.4 to 85.4 with an amino acid sequence of 2 to 4 amino acids, wherein the amino acid sequence allows for formation of a beta-turn, where the numbering of amino acids is IMGT numbering, and where the TCR construct has an increased TCR melting temperature (Tm) as compared to a corresponding TCR construct that does not comprise the two or more stabilizing mutations.

In another aspect, the present disclosure relates to a TCR construct comprising a TCR alpha chain polypeptide and a TCR beta chain polypeptide, the TCR alpha chain polypeptide comprising a variable alpha (Vα) domain and a constant alpha (Cα) domain and the TCR beta chain polypeptide comprising a variable beta (Vβ) domain and a constant beta (Cβ) domain, the TCR construct comprising a combination of amino acid mutations as set forth for any one of the variants shown in Table 2, wherein the numbering of amino acids is IMGT numbering.

In another aspect, the present disclosure relates to a TCR construct comprising a TCR alpha chain polypeptide and a TCR beta chain polypeptide, the TCR alpha chain polypeptide comprising a variable alpha (Vα) domain and a constant alpha (Cα) domain and the TCR beta chain polypeptide comprising a variable beta (Vβ) domain and a constant beta (Cβ) domain, the TCR construct comprising a combination of amino acid mutations as set forth for any one of the variants shown in Table 3, wherein the numbering of amino acids is IMGT numbering.

In another aspect, the present disclosure relates to a TCR fusion protein comprising one or more TCR constructs described herein and a scaffold, wherein at least one of the TCR constructs is fused to the scaffold.

In another aspect, the present disclosure relates to a pharmaceutical composition comprising a TCR construct or TCR fusion protein as described herein and a pharmaceutically acceptable carrier or diluent.

In another aspect, the present disclosure relates to a polynucleotide or set of polynucleotides encoding a TCR construct or TCR fusion protein as described herein.

In another aspect, the present disclosure relates to a method of preparing a TCR construct or TCR fusion protein as described herein comprising transfecting a cell with a polynucleotide or set of polynucleotides encoding the TCR construct or TCR fusion protein, and culturing the cell under conditions suitable for expression of the TCR construct or TCR fusion protein.

In another aspect, the present disclosure relates to a method of treating a disease or disorder in a subject in need thereof, the method comprising administering to the subject an effective amount of a TCR construct or a TCR fusion protein as described herein.

In another aspect, the present disclosure relates to the use of a TCR construct or a TCR fusion protein as described herein in therapy.

### BRIEF DESCRIPTION OF THE DRAWINGS

FIG. 1 presents a schematic representation of exemplary TCR fusion proteins and controls as described herein and in the Examples. (A) control one-armed antibody (OA-scFv); (B) control antibody comprising two scFv arms (Dual scFv); (C) TCR-scFv; (D) One-armed alpha fusion; (E) One-armed alpha fusion-DS; (F) Dual alpha fusion-DS; (G) Dual-fusion; (H) One-armed beta fusion; (I) One-armed beta fusion-DS; (J) Dual beta fusion-DS; (K) Bispecific alpha fusion; (L) Bispecific beta fusion; (M) Chimera Bispecific; (N) Bispecific tandem beta-fusion; (O) 2×1 Bispecific beta-fusion; (P) 3×1 Bispecific beta-fusion; (Q) 4×1 Bispecific C-terminal beta-fusion; (R) 4×1 Bispecific light chain beta-fusion; (S) One-armed alpha fusion, and (T) One-armed beta fusion. The black bar between TRAC and TRBC domains represents an interchain disulfide bond (“DS”).

FIG. 2 presents differential scanning calorimetry (DSC) thermograms for TCR fusion proteins comprising exemplary stabilizing mutations as described herein (v21230: IC Disulfide; v22712: TRBC/6.VAL->LEU+IC Disulfide, and v28881: TRBC/6.VAL->ILE+IC Disulfide). Each peak on the DSC thermogram corresponds to a thermal transition. There are three expected thermal transitions: TCR, CH2 (˜71° C.) and CH3 (˜80° C.). The transition of the TCR includes the TRAV-TRBV and TRAC-TRBC interfaces.

FIG. 3 presents a comparison of the UPLC-SEC traces for an exemplary TCR fusion protein described herein (variant v21230, containing the IC Disulfide) after expression at (A) 37° C., and (B) 32° C. The monodispersed species elute at 2.6 min. The high molecular weight species (aggregates) elute prior to the monodispersed species. The peaks at 2.8 and 3.25 min correspond to the mis-paired anti-CD3 scFv homodimer and unpaired anti-CD3 scFv, respectively.

FIG. 4 presents differential scanning calorimetry (DSC) thermograms showing the change in Tm for an exemplary TCR fusion protein comprising the TRAC-Hinge Disulfide described herein (v22752: IC Disulfide+TRAC-Hinge Disulfide) compared to a TCR fusion protein comprising the IC Disulfide alone (v21230). Each peak on the DSC thermogram corresponds to a thermal transition as described for FIG. 2.

FIG. 5 shows binding of exemplary TCR fusion proteins to target cell surface peptide-MHC complex by flow cytometry, (A) variant v21230 (control comprising the IC Disulfide), and variants v22705, v22707 and v22709; (B) variant v21230 (control comprising the IC Disulfide), and variants v22712, v22716, v22720 and v22722; (C) variant v21230 (control comprising the IC Disulfide), and variants v22729, v22730, v22748 and v22752, and (D) variant v21230 (control comprising the IC Disulfide), and variants v22772, v22837, v22840 and v22842. (See Tables 4.1 and 8.1 for variant descriptions).

FIG. 6 presents the EC₅₀ values measured for binding of exemplary TCR fusion proteins to target cell surface peptide-MHC complex (assessed by flow cytometry). The dashed line corresponds to the EC₅₀ of control variant v21230 which contains the IC Disulfide.

FIG. 7 presents the results of assessment of the avidity effects using a low affinity TCR component in exemplary TCR fusion proteins having different formats. Variants v30972 (bispecific tandem beta-fusion), v30964 (bispecific beta-fusion), v30975 (one-armed beta fusion) and v30968 (2×1 bispecific beta-fusion) comprise a low affinity anti-gp100 TCR component. Variants v29011 (one-armed beta fusion) and v31327 (bispecific beta-fusion) comprise a higher affinity anti-gp100 TCR component. Variant v30968 comprises 2 copies of the TCR component, all other variants comprise one copy of the TCR component.

FIG. 8 presents UPLC-SEC traces for two exemplary TCR fusion proteins (A) variant v32548, which is in a “4×1 tandem” format comprising anti-NY-ESO1 TCRs, and (B) variant v32549, which is in a “4×1 LC” format comprising anti-NY-ESO1 TCRs.

FIG. 9 presents the amino acid sequences of the TCR alpha chain constant region (TRAC; SEQ ID NO:1) and the TCR beta chain constant regions (TRBC1; SEQ ID NO:2 and TRBC2; SEQ ID NO:3), together with the standard IMGT numbering. Differences between the sequences of TRBC1 and TRBC2 are shown in bold italic font.

FIG. 10 shows the cytotoxic effects of TCR fusion proteins having different formats as measured by a T2 T-cell dependent cytotoxicity assay. The TCR fusion proteins comprised a stabilized affinity matured 1G4-33A anti-NY-ESO1 TCR and an anti-CD3 Fab or scFv. Variant v31185 is a control one-armed TCR construct that lacks the anti-CD3 component.

FIG. 11 presents a table of TCR fusion protein variants (Table 2) comprising exemplary combinations of stabilizing mutations.

FIG. 12 presents a table of TCR fusion protein variants (Table 3) comprising exemplary combinations of stabilizing mutations.

### DETAILED DESCRIPTION

The present disclosure relates to stabilized TCR constructs. The TCR constructs comprise a TCR alpha chain polypeptide having a variable alpha (Vα) domain and a constant alpha (Cα) domain and a TCR beta chain polypeptide having a variable beta (Vβ) domain and a constant beta (Cβ) domain and are stabilized by the introduction of stabilizing mutations into the Cα domain and/or the Cβ domain. The stabilizing mutations may include one or more non-naturally occurring interchain disulfide bonds, one or more non-naturally occurring intrachain disulfide bonds, one or more point mutations, one or more loop truncation mutations, or combinations thereof.

In certain embodiments, the stabilizing mutations include the introduction a non-naturally occurring disulfide bond between the Cα domain and the Cβ domain (an interchain disulfide bond), together with one or more additional stabilizing mutations. The additional stabilizing mutations may include additional non-naturally occurring interchain disulfide bonds, non-naturally occurring intrachain disulfide bonds, point mutations, loop truncation mutations, or combinations thereof.

Also disclosed herein are TCR fusion proteins comprising one or more TCR constructs as described herein fused to a scaffold, such as an immunoglobulin (Ig) Fc region. The Ig Fc region may be, for example, an IgG or IgA Fc region.

The TCR constructs and TCR fusion proteins may find use, for example, as therapeutic or diagnostic agents.

### Definitions

Unless defined otherwise, all technical and scientific terms used herein have the same meaning as commonly understood by one of ordinary skill in the art.

As used herein, the term “about” refers to an approximately +/−10% variation from a given value. It is to be understood that such a variation is always included in any given value provided herein, whether or not it is specifically referred to.

The use of the word “a” or “an” when used herein in conjunction with the term “comprising” may mean “one,” but it is also consistent with the meaning of “one or more,” “at least one” and “one or more than one” unless the context clearly dictates otherwise.

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

When two components of a TCR construct or TCR fusion protein described herein are “fused,” it is meant that the components are linked by peptide bonds, either directly or via a peptide linker.

The terms “derived from” and “based on” when used with reference to a recombinant amino acid sequence mean that the recombinant amino acid sequence is substantially identical to the sequence of the corresponding wild-type amino acid sequence. For example, an Ig Fc amino acid sequence that is derived from (or based on) a wild-type Ig Fc sequence is substantially identical (for example, shares at least 90%, at least 95%, at least 96%, at least 97%, at least 98% or at least 99% sequence identity) with the wild-type Ig Fc sequence.

Where a range of values is provided herein, for example where a value is defined as being “between” an upper limit value and a lower limit value, it is understood that the range encompasses both the upper limit value and the lower limit value as well as each intervening value.

It is contemplated that any embodiment discussed herein regarding a TCR construct can be implemented with respect to any fusion protein, method, use or composition disclosed herein.

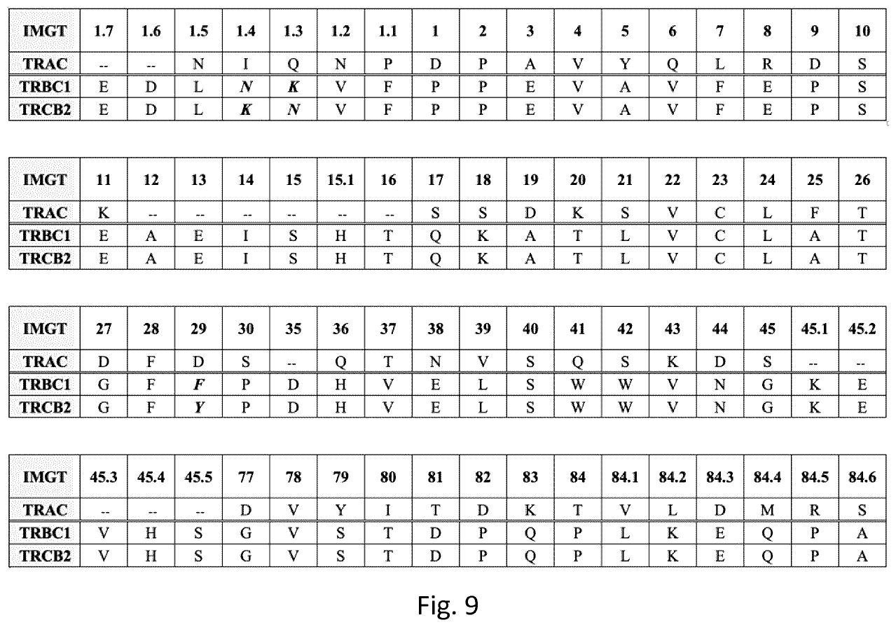

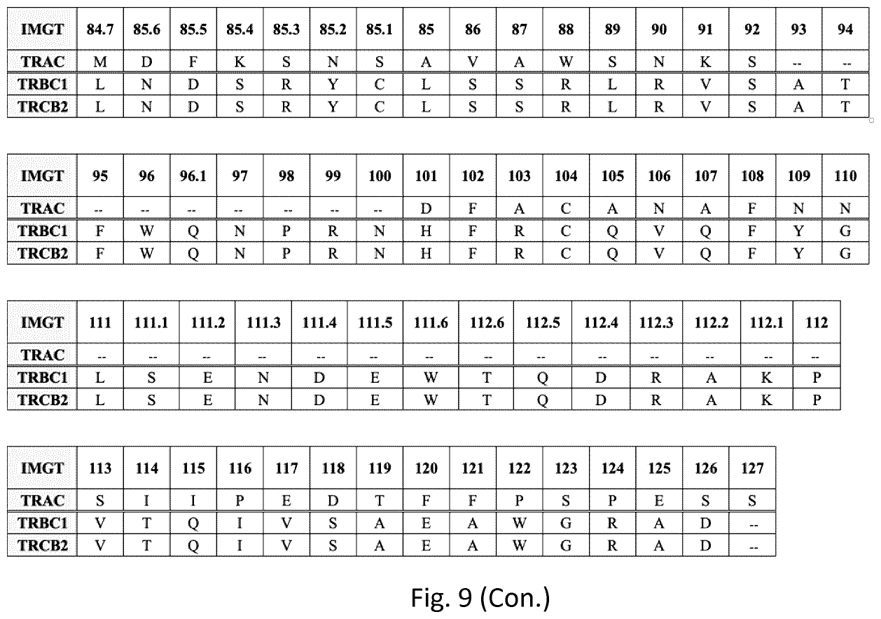

Amino acid residues in the extracellular TCR domains are numbered according to the IMGT numbering system (Lefranc, et al., 2005, Developmental and Comparative Immunology, 29:185-203. See also FIG. 9).

### TCR Constructs

The TCR constructs of the present disclosure are based on an αβTCR heterodimer and comprise a TCR alpha chain polypeptide having a variable alpha (Vα) domain and a constant alpha (Cα) domain and a TCR beta chain polypeptide having a variable beta (Vβ) domain and a constant beta (Cβ) domain. The TCR constructs are stabilized by the introduction of stabilizing mutations into the Cα domain and/or the Cβ domain.

Human wild-type ββTCRs comprise a Cα domain (T-cell receptor alpha constant (TRAC)) and a Cβ domain (either T-cell receptor beta constant 1 (TRBC1) or T-cell receptor beta constant 2 (TRBC2)). The sequences of TRBC1 and TRBC2 differ in only 3 residues: position TRBC/1.4 is Asn in TRBC1 and Lys in TRBC2; position TRBC/1.3 is Lys in TRBC1 and Asn in TRBC2, and position TRBC/29 is Phe in TRBC1 and Tyr in TRBC2. The amino acid sequences of the human TRAC (SEQ ID NO:1), TRBC1 (SEQ ID NO:2) and TRBC2 (SEQ ID NO:3) are shown in FIG. 9.

As used herein, the term “Ca. domain” refers to the amino acid sequence of a TRAC Cα domain and excludes any transmembrane sequence. The Cα domain comprised by the TCR constructs described herein may optionally comprise the naturally-occurring cysteine residue at position TRAC/128. In some embodiments, the Cα domain comprised by the TCR construct has the amino acid sequence of the human TRAC Cα domain as set forth in SEQ ID NO:1 (i.e. ending at position TRAC/127). In some embodiments, for example when the TCR construct comprises a disulfide bond that involves the naturally-occurring cysteine residue at position TRAC/128, the Cα domain comprised by the TCR construct has the amino acid sequence of the human TRAC Cα domain as set forth in SEQ ID NO:4 (i.e. including the cysteine residue at position TRAC/128).

As used herein, the term “Cβ domain” refers to the amino acid sequence of a TRBC Cβ domain and excludes any transmembrane sequence. In some embodiments, the Cβ domain comprised by the TCR construct has the amino acid sequence of the human TRBC1 or TRBC2 Cβ domain ending at position TRBC/126. In some embodiments, the Cβ domain comprised by the TCR construct has the amino acid sequence of the human TRBC1 Cβ domain as set forth in SEQ ID NO:2. In some embodiments, the Cβ domain comprised by the TCR construct has the amino acid sequence of the human TRBC2 Cβ domain as set forth in SEQ ID NO:3. In some embodiments, the Cβ domain comprised by the TCR construct has the amino acid sequence of the human TRBC1 Cβ domain in which position 85.1 has been mutated from cysteine to alanine, as shown in SEQ ID NO:43.

In certain embodiments, the TCR beta chain polypeptide further comprises a cysteine residue at the C-terminus of the Cβ domain that forms a non-naturally occurring disulfide bond with a cysteine residue in the Cα domain of the TCR alpha chain polypeptide as described herein. The cysteine residue at the C-terminus of the Cβ domain of the TCR beta chain polypeptide may be a single amino acid addition or the cysteine residue may be part of a short amino acid extension at the C-terminus of the Cβ domain of the TCR beta chain polypeptide as described herein. In some embodiments, the cysteine residue may be part of a short amino acid extension, for example between 1 and about 10 amino acids in length, at the C-terminus of the Cβ domain of the TCR beta chain polypeptide. In some embodiments, the cysteine residue may be part of a short amino acid extension, for example between 1 and about 10 amino acids in length, at the C-terminus of the Cβ domain of the TCR beta chain polypeptide where the amino acid extension comprises all or a portion, for example 3 or more consecutive amino acids, of the sequence of an IgG1 hinge region, such as EPKSCDKTHT [SEQ ID NO:16], or EPKSCDKTHTCPPCP [SEQ ID NO:21].

The stabilizing mutations introduced into the Cα domain and/or the Cβ domain of the TCR constructs of the present disclosure may include non-naturally occurring interchain disulfide bonds, non-naturally occurring intrachain disulfide bonds, point mutations, loop truncation mutations, and combinations thereof, as described in detail below. The stabilizing mutations comprised by the TCR constructs improve the stability of the TCR construct as compared to a TCR construct that does not comprise the stabilizing mutation(s). Improving the stability of the TCR construct in this context may include improving the thermal stability of the TCR construct, improving the colloidal stability of the TCR construct, or both.

In certain embodiments, the TCR constructs of the present disclosure show an improvement in thermal stability as compared to a corresponding TCR construct that does not comprise the stabilizing mutation(s). Thermal stability of the TCR constructs may be assessed, for example, by measuring the melting temperature (Tm) of the TCR construct. Accordingly, in some embodiments, the TCR constructs have an increased Tm as compared to a corresponding TCR construct that does not comprise the stabilizing mutation(s).

In certain embodiments, the TCR constructs of the present disclosure have a Tm that is increased by 0.5° C. or more as compared to the Tm of a corresponding TCR construct that does not comprise the stabilizing mutation(s). In some embodiments, the TCR constructs have a Tm that is increased by 1° C. or more as compared to the Tm of a corresponding TCR construct that does not comprise the stabilizing mutation(s), for example, by 2° C. or more, 3° C. or more, 4° C. or more, or 5° C. or more, as compared to the Tm of a corresponding TCR construct that does not comprise the stabilizing mutation(s).

In certain embodiments, the TCR constructs of the present disclosure have a Tm that is increased by between 0.5° C. and about 10° C. as compared to the Tm of a corresponding TCR construct that does not comprise the stabilizing mutation(s). In some embodiments, the TCR constructs have a Tm that is increased by between 1° C. and about 10° C. as compared to the Tm of a corresponding TCR construct that does not comprise the stabilizing mutation(s), for example, between 1° C. and about 9° C., between 1° C. and about 8° C., or between 1° C. and about 7° C., as compared to the Tm of a corresponding TCR construct that does not comprise the stabilizing mutation(s). In some embodiments, the TCR constructs have a Tm that is increased by between 2° C. and about 10° C., between 2° C. and about 9° C., between 2° C. and about 8° C., or between 2° C. and about 7° C., as compared to the Tm of a corresponding TCR construct that does not comprise the stabilizing mutation(s).

In certain embodiments, the TCR constructs of the present disclosure comprise a non-naturally occurring disulfide bond between the Cα domain and the Cβ domain (a “first non-naturally occurring interchain disulfide bond”), together with one or more additional stabilizing mutations as described herein and have an increased Tm as compared to a corresponding TCR construct comprising the first non-naturally occurring interchain disulfide bond alone.

In certain embodiments, the TCR constructs comprise a first non-naturally occurring interchain disulfide bond and one or more additional stabilizing mutations and have a Tm that is increased by 0.5° C. or more as compared to the Tm of a corresponding TCR construct comprising the first non-naturally occurring interchain disulfide bond alone, for example, by 1° C. or more, by 2° C. or more, 3° C. or more, 4° C. or more, or 5° C. or more, as compared to the Tm of a corresponding TCR construct comprising the first non-naturally occurring interchain disulfide bond alone.

In certain embodiments, the TCR constructs comprise a first non-naturally occurring interchain disulfide bond and one or more additional stabilizing mutations and have a Tm that is increased by between 0.5° C. and about 10° C. as compared to the Tm of a corresponding TCR construct comprising the first non-naturally occurring interchain disulfide bond alone, for example, by between 1° C. and about 10° C., by between 1° C. and about 9° C., between 1° C. and about 8° C., or between 1° C. and about 7° C., as compared to the Tm of a corresponding TCR construct comprising the first non-naturally occurring interchain disulfide bond alone. In some embodiments, the TCR constructs comprising a first non-naturally occurring interchain disulfide bond and one or more additional stabilizing mutations have a Tm that is increased by between 2° C. and about 10° C., between 2° C. and about 9° C., between 2° C. and about 8° C., or between 2° C. and about 7° C., as compared to the Tm of a corresponding TCR construct comprising the first non-naturally occurring interchain disulfide bond alone.

The Tm of the TCR constructs may be measured, for example, by circular dichroism (CD), differential scanning calorimetry (DSC) or differential scanning fluorimetry (DSF) using standard techniques. In certain embodiments, the TCR constructs have an increased Tm as compared to the stipulated corresponding control TCR construct (for example, a corresponding TCR construct that does not comprise the stabilizing mutation(s) or a corresponding TCR construct comprising a first non-naturally occurring interchain disulfide bond alone), where the Tm is measured by DSC.

In certain embodiments, the TCR constructs of the present disclosure show an improvement in colloidal stability as compared to a corresponding TCR construct that does not comprise the stabilizing mutation(s). Colloidal stability may be assessed, for example, by measuring the amount of high molecular weight (HMW) species (or aggregation) of the TCR construct that occurs during expression of the TCR construct. Accordingly, in some embodiments, the TCR constructs of the present disclosure show decreased amounts of HMW species (aggregation) as compared to a corresponding TCR construct that does not comprise the stabilizing mutation(s) when expressed under the same conditions. In certain embodiments, the TCR constructs comprise a first non-naturally occurring interchain disulfide bond and one or more additional stabilizing mutations and show decreased amounts of HMW species (aggregation) as compared to a corresponding TCR construct comprising the first non-naturally occurring interchain disulfide bond alone when expressed under the same conditions.

The amount of HMW species present in a preparation of a TCR construct may be assessed by various standard techniques known in the art. For example, the amount of HMW species in a preparation of a TCR construct may be assessed by size-exclusion chromatography (SEC), for example using UPLC-SEC, or dynamic light scattering (DLS). In certain embodiments, the TCR constructs show decreased amounts of HMW species as compared to the stipulated corresponding control TCR construct, where the amount of HMW species is assessed by UPLC-SEC.

A non-naturally occurring interchain disulfide bond between cysteine substitutions at positions TRAC/84.THR-TRBC/79.SER that stabilizes soluble αβTCRs has been previously described (Boulter, et al., 2003, PEDS, 16:707-711). This TRAC/84.THR-TRBC/79.SER disulfide bond is referred to herein as the “IC Disulfide.” In certain embodiments, the TCR constructs of the present disclosure have an increased Tm as compared to a corresponding TCR construct comprising the IC Disulfide alone. In certain embodiments, the TCR constructs of the present disclosure comprise a first non-naturally occurring disulfide bond and one or more additional stabilizing mutations as described herein, where the first non-naturally occurring disulfide bond is the IC Disulfide, and have an increased Tm as compared to a corresponding TCR construct comprising the IC Disulfide alone.

In certain embodiments, the TCR constructs of the present disclosure have a Tm that is increased by 0.5° C. or more as compared to the Tm of a corresponding TCR construct comprising the IC Disulfide alone. In some embodiments, the TCR constructs have a Tm that is increased by 1° C. or more as compared to the Tm of a corresponding TCR construct comprising the IC Disulfide alone, for example, by 2° C. or more, 3° C. or more, 4° C. or more, or 5° C. or more, as compared to the Tm of a corresponding TCR construct comprising the IC Disulfide alone.

In certain embodiments, the TCR constructs of the present disclosure have a Tm that is increased by between 0.5° C. and about 10° C. as compared to the Tm of a corresponding TCR construct comprising the IC Disulfide alone, for example, by between 1° C. and about 10° C., by between 1° C. and about 9° C., between 1° C. and about 8° C., or between 1° C. and about 7° C., as compared to the Tm of a corresponding TCR construct comprising the IC Disulfide alone. In some embodiments, the TCR constructs have a Tm that is increased by between 2° C. and about 10° C., between 2° C. and about 9° C., between 2° C. and about 8° C., or between 2° C. and about 7° C., as compared to the Tm of a corresponding TCR construct comprising the IC Disulfide alone.

In certain embodiments, the TCR constructs of the present disclosure show an amount of HMW species that is substantially the same, or decreased, as compared to a corresponding TCR construct comprising the IC Disulfide alone when expressed under the same conditions. By “substantially the same” in the context of amount of HMW species it is meant that the amount of HMW species measured for the test TCR construct is +10% of the amount of HMW species measured for a corresponding TCR construct comprising the IC Disulfide alone when expressed under the same conditions. In certain embodiments, the amount of H/W species is measured by UPLC-SEC.

In certain embodiments, the TCR constructs of the present disclosure comprise the IC Disulfide and one or more additional stabilizing mutations and have one or both of the following properties: (i) an increased Tm as compared to a corresponding TCR construct comprising the IC Disulfide alone, and/or (ii) a decreased amount of HMW species as compared to a corresponding TCR construct comprising the IC Disulfide alone when expressed under the same conditions.

In certain embodiments, the TCR constructs of the present disclosure comprise a first non-naturally occurring interchain disulfide bond and one or more additional stabilizing mutations and have one or both of the following properties: (i) an increased Tm as compared to a corresponding TCR construct comprising the first non-naturally occurring interchain disulfide bond alone, and/or (ii) a decreased amount of HMW species as compared to a corresponding TCR construct comprising the first non-naturally occurring interchain disulfide bond alone when expressed under the same conditions.

### Stabilizing Mutations

The stabilizing mutations comprised by the TCR constructs of the present disclosure were identified by an iterative process of structure and computational guided design and experimental screening as described herein. In silico modelling using a TCR model based on a Protein Database (PDB) structure was employed to identify mutation designs in the TRAC and TRBC domains that could potentially improve the thermal and/or colloidal stability of the TCR, and these were subsequently tested experimentally.

Mutation designs identified by this approach that successfully improved the thermal and/or colloidal stability of the TCR constructs included disulfide bonds (non-naturally occurring interchain disulfide bonds and/or non-naturally occurring intrachain disulfide bonds), point mutations, loop truncation mutations, and combinations thereof.

### I. Disulfide Bonds

Stabilizing disulfide bonds identified by the above approach include non-naturally occurring interchain disulfide bonds and non-naturally occurring intrachain disulfide bonds. In this context, a non-naturally occurring interchain disulfide bond is a disulfide bond between a cysteine residue in the Cα domain of the TCR construct and a cysteine residue in the Cβ domain of the TCR construct that does not occur in a wild-type TCR. One or both of the cysteine residues of the non-naturally occurring interchain disulfide bond are introduced by substitution of the wild-type residue at a given position with a cysteine residue (a “cysteine residue substitution” or “cysteine substitution) or by addition of a cysteine residue at the C-terminus of the Cα domain or the Cβ domain.

A non-naturally occurring intrachain disulfide bond is a disulfide bond between two cysteine residues in the Cα domain of the TCR construct or between two cysteine residues in the Cβ domain of the TCR construct that does not occur in a wild-type TCR. One or both of the cysteine residues of the non-naturally occurring intrachain disulfide bond is introduced by substitution of the wild-type residue at a given position with a cysteine residue (a “cysteine residue substitution” or “cysteine substitution) or by addition of a cysteine residue at the C-terminus of the Cα domain or the Cβ domain.

### a) Interchain Disulfide Bonds

In certain embodiments, the TCR constructs of the present disclosure include at least one non-naturally occurring interchain disulfide bond.

In certain embodiments, non-naturally occurring interchain disulfide bonds that may be comprised by the TCR constructs include a “hinge disulfide bond.” A “hinge disulfide bond,” as used herein, refers to a disulfide bond formed between a cysteine residue added at the C-terminus of the Cβ domain of the TCR beta chain polypeptide and a cysteine residue in the Cα domain of the TCR alpha chain polypeptide.

The cysteine residue added at the C-terminus of the Cβ domain of the TCR beta chain polypeptide may be a single amino acid addition or the cysteine residue may be part of a short amino acid extension at the C-terminus of the Cβ domain of the TCR beta chain polypeptide. In those embodiments in which the cysteine residue is added at the C-terminus of the Cβ domain of the TCR beta chain polypeptide as part of an amino acid extension, the amino acid extension is typically 10 amino acids or less in length. In some embodiments, the cysteine residue added at the C-terminus of the Cβ domain of the TCR beta chain polypeptide is part of an amino acid extension that is 10 amino acids or less in length, for example, 9 amino acids or less in length, 8 amino acids or less in length, 7 amino acids or less in length, 6 amino acids or less in length, or 5 amino acids or less in length.

In some embodiments, the cysteine residue added at the C-terminus of the Cβ domain of the TCR beta chain polypeptide is part of an amino acid extension that is between 1 and about 10 amino acids in length. In some embodiments, the cysteine residue added at the C-terminus of the Cβ domain of the TCR beta chain polypeptide is part of an amino acid extension that is between 1 and about 9 amino acids in length, for example, between 1 and about 8 amino acids in length, between 1 and about 7 amino acids in length, between 1 and about 6 amino acids in length, or between 1 and about 5 amino acids in length. In some embodiments, the cysteine residue added at the C-terminus of the Cβ domain of the TCR beta chain polypeptide is part of an amino acid extension that is between 2 and about 10 amino acids in length, for example, between 2 and about 9 amino acids in length, for example, between 2 and about 8 amino acids in length, between 2 and about 7 amino acids in length, between 2 and about 6 amino acids in length, or between 2 and about 5 amino acids in length.

In certain embodiments, the amino acid extension comprises a cysteine residue and one or two “linkers” where the linker(s) are composed of amino acids other than cysteine. Thus, in some embodiments, the Cβ domain of the TCR beta chain polypeptide comprises an amino acid extension at the C-terminus that has one of the following structures (from N-terminus to C-terminus):
Linker-Cys,
Cys-Linker or
Linker-Cys-Linker.

When present, the linker allows the cysteine residue of the amino acid extension to assume the correct conformation relative to its cognate cysteine residue in the Cα domain of the TCR alpha chain polypeptide such that the desired disulfide bond is formed. The linker peptide may have one of a number of amino acid sequences known in the art to function successfully as a linker or spacer in polypeptide and protein sequences.

In some embodiments, the linker peptide may comprise the following amino acid residues: Gly, Ser, Ala or Thr, or a combination thereof. Examples of useful linkers include, but are not limited to, glycine-serine linkers, such as (GS)ₙ, (GSGGS)ₙ, (GGGGS)ₙ, and (GGGS)ₙ (where n is an integer between 1 and 4), as well as glycine-alanine linkers and alanine-serine linkers having similar configurations.

In some embodiments, the amino acid extension may comprise all or a portion of a hinge region sequence from an immunoglobulin or from a TCR. In such embodiments, the cysteine residue included in the amino acid extension may occur naturally in the hinge region sequence or it may be introduced by amino acid substitution. Non-limiting examples of hinge region sequences, of which all or a portion (for example, at least 2, 3, 4, 5, 6 or more contiguous amino acids) may be comprised by the amino acid extension, include: ESSCDVKLVEKSFET (SEQ ID NO:5) (TCRα); DCGFTS (SEQ ID NO:6) (TCRβ); DVITMDPKDNCSKDAN (SEQ ID NO:7) (TCRγ); DHVKPKETENTKQPSKSCHKPK (SEQ ID NO:8) (TCRδ); EPKSCDKTHTCPPCP (SEQ ID NO:9) (IgG1); ERKCCVECPPCP (SEQ ID NO:10) (IgG2); ELKTPLGDTTHTCPRCP (SEQ ID NO:11) (IgG3-H1); EPKSCDTPPPCPRCP (SEQ ID NO:12) (IgG3-H2, IgG3-H3 and IgG3-H4); ESKYGPPCPSCP (SEQ ID NO:13) (IgG4); VPPPPP (SEQ ID NO:14) (IgA2).

Immunoglobulin hinge region sequences may be divided into “upper,” “core” and “lower” hinge regions. For example, for IgG1, the full hinge region sequence is: EPKSCDKTHTCPPCPAPELLGG (SEQ ID NO:15). The upper hinge region of the IgG hinge is generally defined as extending from Glu216 to Thr225 (EU numbering) (i.e. EPKSCDKTHT (SEQ ID NO:16)), the core hinge region is generally defined as extending from Cys226 to Pro230 (EU numbering) (i.e. CPPCP (SEQ ID NO:17)), and the lower hinge region is generally defined as extending from Ala231 to Pro238 (EU numbering) (i.e. APELLGG (SEQ ID NO:18)) (see, Burton, 1985, Molec. Immunol., 22:161-206).

In certain embodiments, the amino acid extension comprises all or a portion of an immunoglobulin hinge region sequence. In some embodiments, the amino acid extension comprises all or a portion of an immunoglobulin upper and/or core hinge region sequence. In some embodiments, the amino acid extension comprises all or a portion of an immunoglobulin upper hinge region sequence.

In some embodiments, the amino acid extension comprises all or a portion of an IgG1 hinge region sequence. In some embodiments, the amino acid extension comprises all or a portion of an IgG1 upper and/or core hinge region sequence. In some embodiments, the amino acid extension comprises all or a portion of an IgG1 upper hinge region sequence. In some embodiments, the amino acid extension comprises the sequence: EPKSC [SEQ ID NO:19]. In some embodiments, the amino acid extension comprises the sequence: EPKSCDKTHT [SEQ ID NO:16].

The cysteine residue in the Cα domain of the TCR alpha chain polypeptide that forms the hinge disulfide with the cysteine residue added at the C-terminus of the Cβ domain of the TCR beta chain polypeptide may be a naturally occurring cysteine residue (for example, the naturally occurring cysteine residue at position TRAC 128) or it may be a cysteine substitution at a position proximate to the C-terminus of the TCR alpha chain polypeptide. By “proximate to” in this context, it is meant within 10 amino acids of the C-terminus of the TCR alpha chain polypeptide.

In certain embodiments, the cysteine residue in the Cα domain of the TCR alpha chain polypeptide that forms the hinge disulfide with the cysteine residue added at the C-terminus of the Cβ domain of the TCR beta chain polypeptide is the naturally occurring cysteine residue at position TRAC 128.

In certain embodiments, the TCR construct comprises a non-naturally occurring interchain disulfide bond that is formed between:
i) a cysteine residue comprised by an amino acid extension at the C-terminus of the Cβ domain of the TCR beta chain polypeptide, where the amino acid extension is between 1 and about 10 amino acids in length, and
ii) the naturally occurring cysteine residue at position TRAC 128 in the Cα domain of the TCR alpha chain polypeptide.

In certain embodiments, the TCR construct comprises a non-naturally occurring interchain disulfide bond that is formed between:
i) a cysteine residue in an amino acid extension at the C-terminus of the Cβ domain of the TCR beta chain polypeptide, where the amino acid extension has the sequence: EPKSC [SEQ ID NO:19] or EPKSCDKTHT [SEQ ID NO:16], and
ii) the naturally occurring cysteine residue at position TRAC 128 in the Cα domain of the TCR alpha chain polypeptide.

Other non-naturally occurring interchain disulfide bonds that may be comprised by the TCR constructs include:
i) a disulfide bond between cysteine residue substitutions at positions TRAC 84.2 and TRBC 79;
ii) a disulfide bond between cysteine residue substitutions at positions TRAC 122 and TRBC 12;
iii) a disulfide bond between cysteine residue substitutions at positions TRAC 124 and TRBC 11;
iv) a disulfide bond between cysteine residue substitutions at positions TRAC 125 and TRBC 11;
v) a disulfide bond between cysteine residue substitutions at positions TRAC 126 and TRBC 11;
vi) a disulfide bond between cysteine residue substitutions at positions TRAC 127 and TRBC 11, and
vii) a disulfide bond between cysteine residue substitutions at positions TRAC 128 and TRBC 11.

In certain embodiments, non-naturally occurring interchain disulfide bonds comprised by the TCR constructs may include the IC Disulfide (i.e. a disulfide bond between cysteine residue substitutions at position TRAC 84 in the TCR alpha chain polypeptide and position TRBC 79 in the TCR beta chain polypeptide).

In certain embodiments, the TCR construct comprises a non-naturally occurring interchain disulfide bond selected from:
(a) a disulfide bond formed between i) a cysteine residue comprised by an amino acid extension at the C-terminus of the Cβ domain of the TCR beta chain polypeptide, where the amino acid extension is between 1 and about 10 amino acids in length (for example, an amino acid extension comprising all or a portion of a hinge region sequence from an immunoglobulin or from a TCR, such as an amino acid extension having the sequence: EPKSC [SEQ ID NO:19] or EPKSCDKTHT [SEQ ID NO:16]), and ii) the naturally occurring cysteine residue at position TRAC 128 in the Cα domain of the TCR alpha chain polypeptide;
b) a disulfide bond between cysteine residue substitutions at positions TRAC 84 and TRBC 79;
c) a disulfide bond between cysteine residue substitutions at positions TRAC 84.2 and TRBC 79;
d) a disulfide bond between cysteine residue substitutions at positions TRAC 122 and TRBC 12;
e) a disulfide bond between cysteine residue substitutions at positions TRAC 124 and TRBC 11;
f) a disulfide bond between cysteine residue substitutions at positions TRAC 125 and TRBC 11;
g) a disulfide bond between cysteine residue substitutions at positions TRAC 126 and TRBC 11;
h) a disulfide bond between cysteine residue substitutions at positions TRAC 127 and TRBC 11, and
i) a disulfide bond between cysteine residue substitutions at positions TRAC 128 and TRBC 11.

In certain embodiments, the TCR construct comprises a non-naturally occurring interchain disulfide bond selected from:
(a) a disulfide bond formed between i) a cysteine residue comprised by an amino acid extension at the C-terminus of the Cβ domain of the TCR beta chain polypeptide, where the amino acid extension is between 1 and about 10 amino acids in length (for example, an amino acid extension comprising all or a portion of a hinge region sequence from an immunoglobulin or from a TCR, such as an amino acid extension having the sequence: EPKSC [SEQ ID NO:19] or EPKSCDKTHT [SEQ ID NO:16]), and ii) the naturally occurring cysteine residue at position TRAC 128 in the Cα domain of the TCR alpha chain polypeptide;
b) a disulfide bond between cysteine residue substitutions at positions TRAC 84.2 and TRBC 79;
c) a disulfide bond between cysteine residue substitutions at positions TRAC 124 and TRBC 11;
d) a disulfide bond between cysteine residue substitutions at positions TRAC 125 and TRBC 11;
e) a disulfide bond between cysteine residue substitutions at positions TRAC 126 and TRBC 11;
f) a disulfide bond between cysteine residue substitutions at positions TRAC 127 and TRBC 11, and
g) a disulfide bond between cysteine residue substitutions at positions TRAC 128 and TRBC 11.

### b) Intrachain Disulfide Bonds

Intrachain disulfide bonds that may be included in the TCR constructs as stabilizing mutations include disulfide bonds between two cysteine residues in the Cα domain of the TCR construct and disulfide bonds between two cysteine residues in the Cβ domain of the TCR construct. Typically, at least one of the cysteine residues that make up the intrachain disulfide bond is a cysteine substitution of a naturally occurring residue in the Cα domain or the Cβ domain.

In certain embodiments, the TCR constructs of the present disclosure comprise one or more intrachain disulfide bonds. In some embodiments, the TCR constructs comprise an intrachain disulfide bond between two cysteine residues in the Cα domain of the TCR construct. In some embodiments, the TCR constructs comprise an intrachain disulfide bond between two cysteine residues in the Cα domain of the TCR construct where both cysteine residues involved in the disulfide bond are cysteine substitutions.

Non-naturally occurring intrachain disulfide bonds that may be included in the TCR constructs in certain embodiments include: i) a disulfide bond between cysteine residue substitutions at positions TRAC 39 and TRAC 85, and ii) a disulfide bond between cysteine residue substitutions at positions TRAC 26 and TRAC 85.1.

In certain embodiments in which the TCR constructs of the present disclosure comprise an interchain disulfide bond, the TCR construct may also comprise one or more intrachain disulfide bonds as additional stabilizing mutations.

### II. Point Mutations and Loop Truncation Mutations

A number of stabilizing point and loop truncation mutations were identified by the approach outlined above and described in the Examples. The TCR constructs of the present disclosure may include one or more of these stabilizing point mutations and/or loop truncation mutations.

A “point mutation,” as used herein, refers to a substitution of an amino acid that occurs in the wild-type sequence with a different amino acid. In certain embodiments, the TCR constructs comprise one or more stabilizing point mutations which are amino acid substitutions at one or more of the following positions: TRAC 4, TRAC 26, TRAC 39, TRAC 85, TRAC 105, TRAC 120, TRBC 6, TRBC 36, TRBC 86 and TRBC 45.3.

In certain embodiments, the TCR constructs comprise one or more stabilizing point mutations selected from:
i) an amino acid substitution at position TRAC 4 from Val to Ala, Thr, Ile, Leu or Met;
ii) an amino acid substitution at position TRAC 26 from Thr to Ala, Val, Ile, Leu or Met;
iii) an amino acid substitution at position TRAC 39 from Val to Ala, Thr, Ile, Leu or Met;
iv) an amino acid substitution at position TRAC 85 from Ala to Ser, Thr, Val, Ile or Met;
v) an amino acid substitution at position TRAC 105 from Ala to Ser, Thr, Glu, Gln, Asp, Asn, His, Lys or Arg;
vi) an amino acid substitution at position TRAC 120 from Phe to Tyr or His;
vii) an amino acid substitution at position TRBC 6 from Val to Ala, Thr, Ile, Leu or Met;
viii) an amino acid substitution at position TRBC 36 from His to Phe, Tyr or Trp;
ix) an amino acid substitution at position TRBC 86 from Ser to Ala or Thr, and
x) an amino acid substitution at position TRBC 45.3 from Val to Ser, Thr, Glu, Gln, Asp, Asn, His, Lys or Arg.

In certain embodiments, the amino acid substitution at position TRAC 4 is from Val to Ile. In certain embodiments, the amino acid substitution at position TRAC 26 is from Thr to Ile. In certain embodiments, the amino acid substitution at position TRAC 39 is from Val to Ile. In certain embodiments, the amino acid substitution at position TRAC 85 is from Ala to Val. In certain embodiments, the amino acid substitution at position TRAC 105 is from Ala to Ser. In certain embodiments, the amino acid substitution at position TRAC 120 is from Phe to Tyr. In certain embodiments, the amino acid substitution at position TRBC 6 is from Val to Ile or Leu. In certain embodiments, the amino acid substitution at position TRBC 36 is from His to Phe. In certain embodiments, the amino acid substitution at position TRBC 86 is from Ser to Thr. In certain embodiments, the amino acid substitution at position TRBC 45.3 is from Val to Thr.

In certain embodiments, the TCR constructs comprise one or more stabilizing point mutations selected from:
i) an amino acid substitution at position TRAC 4 from Val to Ile;
ii) an amino acid substitution at position TRAC 26 from Thr to Ile;
iii) an amino acid substitution at position TRAC 39 from Val to Ile;
iv) an amino acid substitution at position TRAC 85 from Ala to Val;
v) an amino acid substitution at position TRAC 105 from Ala to Ser;
vi) an amino acid substitution at position TRAC 120 from Phe to Tyr;
vii) an amino acid substitution at position TRBC 6 from Val to Ile or Leu;
viii) an amino acid substitution at position TRBC 36 from His to Phe;
ix) an amino acid substitution at position TRBC 86 from Ser to Thr, and
x) an amino acid substitution at position TRBC 45.3 from Val to Thr.

A “loop truncation mutation,” as used herein, refers to a mutation that shortens a naturally occurring loop in the wild-type TCR sequence by deletion of one or more amino acids in the loop, or by replacement of all or a part of the loop with a shorter sequence of amino acids. In certain embodiments, the TCR constructs may comprise a loop truncation mutation that shortens the DE loop in the Cβ domain of the TCR construct. The DE loop in the Cβ domain is 13 amino acids in length and extends from position TRBC 84.1 to position TRBC 85.1. In some embodiments, the loop truncation mutation shortens the DE loop in the Cβ domain of the TCR construct by between 1 and 10 amino acids. In some embodiments, the loop truncation mutation shortens the DE loop in the Cβ domain of the TCR construct by between 1 and 9 amino acids, between 1 and 8 amino acids, between 1 and 7 amino acids, between 1 and 6 amino acids, between 1 and 5 amino acids or between 1 and 4 amino acids.

In certain non-human species, the DE loop in the TCR Cβ domain differs in amino acid sequence to the DE loop in the human TCR Cβ domain and notably is 3 or 4 residues shorter than the human TCR DE loop. Accordingly, in certain embodiments, the TCR constructs comprise a loop truncation mutation that is a deletion of one or more amino acids, for example, between 1 and 4 consecutive amino acids, or between 1 and 3 consecutive amino acids, of the DE loop in the Cβ domain of the TCR construct. In some embodiments, the TCR construct comprises a loop truncation mutation that is a deletion of the amino acids at positions TRBC 84.5 to 85.6.

In certain embodiments, the TCR constructs comprise a loop truncation mutation that is a replacement of all or a part of the DE loop in the Cβ domain of the TCR construct with a shorter sequence of amino acids such that the DE loop is shortened by between 1 and 8 amino acids, for example, between 1 and 7 amino acids or between 1 and 6 amino acids.

In some embodiments, the TCR constructs comprise a loop truncation mutation that is a replacement of between 5 and 13 consecutive amino acids of the DE loop with a shorter sequence of amino acids. In some embodiments, the TCR constructs comprise a loop truncation mutation that is a replacement of between 5 and 10 consecutive amino acids, for example, between 5 and 9, between 5 and 8, or between 5 and 7 consecutive amino acids, of the DE loop with a shorter sequence of amino acids.

In some embodiments, the shorter amino acid sequence is selected such that it provides a beta-turn motif, either alone or in combination with flanking amino acids at the site of insertion, and thus allows for formation of a beta-turn. Amino acid sequences that allow for the formation of a beta-turn can be readily identified using, for example, sequence analyzing software and programs known in the art. In such embodiments, the shorter amino acid sequence is typically between 2 and 4 amino acids in length. In some embodiments, the TCR constructs comprise a loop truncation mutation that is a replacement of between 5 and 13 consecutive amino acids, for example, between 5 and 10 consecutive amino acids, between 5 and 9, between 5 and 8, or between 5 and 7 consecutive amino acids, of the DE loop with a shorter sequence of amino acids that allows for formation of a beta-turn. In some embodiments, the TCR constructs comprise a loop truncation mutation that is a replacement of between 5 and 13 consecutive amino acids, for example, between 5 and 10 consecutive amino acids, between 5 and 9, between 5 and 8, or between 5 and 7 consecutive amino acids, of the DE loop with a shorter sequence of between 2 and 4 amino acids that allows for formation of a beta-turn. In some embodiments, the TCR construct comprises a loop truncation mutation that is a replacement of the amino acids at positions TRBC 84.4 to 85.4 with a shorter sequence of between 2 and 4 amino acids that allows for formation of a beta-turn.

In some embodiments, the TCR construct comprises a loop truncation mutation that is a replacement of the amino acids at positions TRBC 84.4 to 85.4 with the amino acids Gly-Asn.

In certain embodiments, the TCR constructs comprise one or more loop truncation mutations selected from:
a deletion of between 1 and 4 consecutive amino acids of the DE loop in the Cβ domain of the TCR construct, and
a replacement of between 5 and 7 consecutive amino acids of the DE loop in the Cβ domain of the TCR construct with an amino acid sequence of between 2 and 4 amino acids that allows for formation of a beta-turn.

In certain embodiments, the TCR constructs comprise one or more loop truncation mutations selected from:
a deletion of between 1 and 4 consecutive amino acids of the DE loop in the Cβ domain of the TCR construct, and
a replacement of the amino acids at positions TRBC 84.4 to 85.4 with an amino acid sequence of between 2 and 4 amino acids that allows for formation of a beta-turn.

In certain embodiments, the TCR constructs comprise one or more loop truncation mutations selected from:
a deletion of the amino acids at positions TRBC 84.5 to 85.6, and
a replacement of the amino acids at positions TRBC 84.4 to 85.4 with the amino acids Gly-Asn.

### III. Combinations of Stabilizing Mutations

The TCR constructs of the present disclosure may comprise one or a combination of the stabilizing mutations described herein.

In certain embodiments, the TCR constructs comprise one or more of the following stabilizing mutations:
a) a disulfide bond formed between: i) a cysteine residue comprised by an amino acid extension at the C-terminus of the Cβ domain of the TCR beta chain polypeptide, where the amino acid extension is between 1 and about 10 amino acids in length, and ii) the naturally occurring cysteine residue at position TRAC 128 in the Cα domain of the TCR alpha chain polypeptide;
b) a disulfide bond between cysteine residue substitutions at positions TRAC 84.2 and TRBC 79;
c) a disulfide bond between cysteine residue substitutions at positions TRAC 124 and TRBC 11;
d) a disulfide bond between cysteine residue substitutions at positions TRAC 125 and TRBC 11;
e) a disulfide bond between cysteine residue substitutions at positions TRAC 126 and TRBC 11;
f) a disulfide bond between cysteine residue substitutions at positions TRAC 127 and TRBC 11;
g) a disulfide bond between cysteine residue substitutions at positions TRAC 128 and TRBC 11;
h) a disulfide bond between cysteine residue substitutions at positions TRAC 39 and TRAC 85;
i) a disulfide bond between cysteine residue substitutions at positions TRAC 26 and TRAC 85.1;
j) an amino acid substitution at position TRAC 4 from Val to Ala, Thr, Ile, Leu or Met;
k) an amino acid substitution at position TRAC 26 from Thr to Ala, Val, Ile, Leu or Met;
l) an amino acid substitution at position TRAC 39 from Val to Ala, Thr, Ile, Leu or Met;
m) an amino acid substitution at position TRAC 85 from Ala to Ser, Thr, Val, Ile or Met;
n) an amino acid substitution at position TRAC 105 from Ala to Ser, Thr, Glu, Gln, Asp, Asn, His, Lys or Arg;
o) an amino acid substitution at position TRAC 120 from Phe to Tyr or His;
p) an amino acid substitution at position TRBC 6 from Val to Ala, Thr, Ile, Leu or Met;
q) an amino acid substitution at position TRBC 36 from His to Phe, Tyr or Trp;
r) an amino acid substitution at position TRBC 86 from Ser to Ala or Thr;
s) an amino acid substitution at position TRBC 45.3 from Val to Ser, Thr, Glu, Gln, Asp, Asn, His, Lys or Arg;
t) a deletion of between 1 and 4 consecutive amino acids of the DE loop in the Cβ domain of the TCR construct, and
u) a replacement of the amino acids at positions TRBC 84.4 to 85.4 with an amino acid sequence of between 2 and 4 amino acids that allows for formation of a beta-turn.

In some embodiments, the amino acid extension at the C-terminus of the Cβ domain of the TCR beta chain polypeptide comprises all or a portion of a hinge region sequence from an immunoglobulin or from a TCR. In some embodiments, the amino acid extension at the C-terminus of the Cβ domain of the TCR beta chain polypeptide has the sequence: EPKSC [SEQ ID NO:19] or EPKSCDKTHT [SEQ ID NO:16].

In some embodiments, the amino acid substitution at position TRAC 4 is from Val to Ile. In some embodiments, the amino acid substitution at position TRAC 26 is from Thr to Ile. In some embodiments, the amino acid substitution at position TRAC 39 is from Val to Ile. In some embodiments, the amino acid substitution at position TRAC 85 is from Ala to Val. In some embodiments, the amino acid substitution at position TRAC 105 is from Ala to Ser. In some embodiments, the amino acid substitution at position TRAC 120 is from Phe to Tyr. In some embodiments, the amino acid substitution at position TRBC 6 is from Val to Leu or Ile. In some embodiments, the amino acid substitution at position TRBC 36 is from His to Phe. In some embodiments, the amino acid substitution at position TRBC 86 is from Ser to Thr. In some embodiments, the amino acid substitution at position TRBC 45.3 is from Val to Thr.

In some embodiments, the deletion of between 1 and 4 consecutive amino acids of the DE loop in the Cβ domain of the TCR construct is a deletion of the amino acids at positions TRBC 84.5 to 85.6.

In some embodiments, the replacement of the amino acids at positions TRBC 84.4 to 85.4 is with the amino acids Gly-Asn.

Combinations of any of the foregoing embodiments for TCR constructs are also contemplated and each combination forms a separate embodiment for the purposes of the present disclosure.

In some embodiments, the TCR constructs comprise one or more of the following stabilizing mutations:
a) a disulfide bond formed between: i) a cysteine residue comprised by an amino acid extension at the C-terminus of the Cβ domain of the TCR beta chain polypeptide, where the amino acid extension has the sequence: EPKSC [SEQ ID NO:19] or EPKSCDKTHT [SEQ ID NO:16], and ii) the naturally occurring cysteine residue at position TRAC 128 in the Cα domain of the TCR alpha chain polypeptide;
b) a disulfide bond between cysteine residue substitutions at positions TRAC 84.2 and TRBC 79;
c) a disulfide bond between cysteine residue substitutions at positions TRAC 124 and TRBC 11;
d) a disulfide bond between cysteine residue substitutions at positions TRAC 125 and TRBC 11;
e) a disulfide bond between cysteine residue substitutions at positions TRAC 126 and TRBC 11;
f) a disulfide bond between cysteine residue substitutions at positions TRAC 127 and TRBC 11;
g) a disulfide bond between cysteine residue substitutions at positions TRAC 128 and TRBC 11;
h) a disulfide bond between cysteine residue substitutions at positions TRAC 39 and TRAC 85;
i) a disulfide bond between cysteine residue substitutions at positions TRAC 26 and TRAC 85.1;
j) an amino acid substitution at position TRAC 4 from Val to Ile;
k) an amino acid substitution at position TRAC 26 from Thr to Ile;
l) an amino acid substitution at position TRAC 39 from Val to Ile;
m) an amino acid substitution at position TRAC 85 from Ala to Val;
n) an amino acid substitution at position TRAC 105 from Ala to Ser;
o) an amino acid substitution at position TRAC 120 from Phe to Tyr;
p) an amino acid substitution at position TRBC 6 from Val to Leu or Ile;
q) an amino acid substitution at position TRBC 36 from His to Phe;
r) an amino acid substitution at position TRBC 86 from Ser to Thr;
s) an amino acid substitution at position TRBC 45.3 from Val to Thr;
t) a deletion of the amino acids at positions TRBC 84.5 to 85.6, and
u) a replacement of the amino acids at positions TRBC 84.4 to 85.4 with the amino acids Gly-Asn.

In certain embodiments, the TCR constructs of the present disclosure comprise a combination of two or more of the stabilizing mutations described herein. Combinations of stabilizing mutations that may be comprised by the TCR constructs include combinations of non-naturally occurring interchain disulfide bonds, combinations of non-naturally occurring intrachain disulfide bonds, combinations of point mutations and loop truncation mutations, and combinations comprising different categories of stabilizing mutations.

In certain embodiments, the TCR constructs comprise two or more of the following stabilizing mutations:
a) a disulfide bond formed between: i) a cysteine residue comprised by an amino acid extension at the C-terminus of the Cβ domain of the TCR beta chain polypeptide, where the amino acid extension is between 1 and about 10 amino acids in length, and ii) the naturally occurring cysteine residue at position TRAC 128 in the Cα domain of the TCR alpha chain polypeptide;
b) a disulfide bond between cysteine residue substitutions at positions TRAC 84 and TRBC 79;
c) a disulfide bond between cysteine residue substitutions at positions TRAC 84.2 and TRBC 79;
d) a disulfide bond between cysteine residue substitutions at positions TRAC 122 and TRBC 12;
e) a disulfide bond between cysteine residue substitutions at positions TRAC 124 and TRBC 11;
f) a disulfide bond between cysteine residue substitutions at positions TRAC 125 and TRBC 11;
g) a disulfide bond between cysteine residue substitutions at positions TRAC 126 and TRBC 11;
h) a disulfide bond between cysteine residue substitutions at positions TRAC 127 and TRBC 11;
i) a disulfide bond between cysteine residue substitutions at positions TRAC 128 and TRBC 11;
j) a disulfide bond between cysteine residue substitutions at positions TRAC 39 and TRAC 85;
k) a disulfide bond between cysteine residue substitutions at positions TRAC 26 and TRAC 85.1;
l) an amino acid substitution at position TRAC 4 from Val to Ala, Thr, Ile, Leu or Met;
m) an amino acid substitution at position TRAC 26 from Thr to Ala, Val, Ile, Leu or Met;
n) an amino acid substitution at position TRAC 39 from Val to Ala, Thr, Ile, Leu or Met;
o) an amino acid substitution at position TRAC 85 from Ala to Ser, Thr, Val, Ile or Met;
p) an amino acid substitution at position TRAC 105 from Ala to Ser, Thr, Glu, Gln, Asp, Asn, His, Lys or Arg;
q) an amino acid substitution at position TRAC 120 from Phe to Tyr or His;
r) an amino acid substitution at position TRBC 6 from Val to Ala, Thr, Ile, Leu or Met;
s) an amino acid substitution at position TRBC 36 from His to Phe, Tyr or Trp;
t) an amino acid substitution at position TRBC 86 from Ser to Ala or Thr;
u) an amino acid substitution at position TRBC 45.3 from Val to Ser, Thr, Glu, Gln, Asp, Asn, His, Lys or Arg;
v) a deletion of between 1 and 4 consecutive amino acids of the DE loop in the Cβ domain of the TCR construct, and
w) a replacement of the amino acids at positions TRBC 84.4 to 85.4 with an amino acid sequence of between 2 and 4 amino acids that allows for formation of a beta-turn.

In some embodiments, the amino acid extension at the C-terminus of the Cβ domain of the TCR beta chain polypeptide comprises all or a portion of a hinge region sequence from an immunoglobulin or from a TCR. In some embodiments, the amino acid extension at the C-terminus of the Cβ domain of the TCR beta chain polypeptide has the sequence: EPKSC [SEQ ID NO:19] or EPKSCDKTHT [SEQ ID NO:16].

In some embodiments, the amino acid substitution at position TRAC 4 is from Val to Ile. In some embodiments, the amino acid substitution at position TRAC 26 is from Thr to Ile. In some embodiments, the amino acid substitution at position TRAC 39 is from Val to Ile. In some embodiments, the amino acid substitution at position TRAC 85 is from Ala to Val. In some embodiments, the amino acid substitution at position TRAC 105 is from Ala to Ser. In some embodiments, the amino acid substitution at position TRAC 120 is from Phe to Tyr. In some embodiments, the amino acid substitution at position TRBC 6 is from Val to Leu or Ile. In some embodiments, the amino acid substitution at position TRBC 36 is from His to Phe. In some embodiments, the amino acid substitution at position TRBC 86 is from Ser to Thr. In some embodiments, the amino acid substitution at position TRBC 45.3 is from Val to Thr.

In some embodiments, the deletion of between 1 and 4 consecutive amino acids of the DE loop in the Cβ domain of the TCR construct is a deletion of the amino acids at positions TRBC 84.5 to 85.6.

In some embodiments, the replacement of the amino acids at positions TRBC 84.4 to 85.4 is with the amino acids Gly-Asn.

Combinations of any of the foregoing embodiments for TCR constructs are also contemplated and each combination forms a separate embodiment for the purposes of the present disclosure.

In some embodiments, the TCR constructs comprise two or more of the following stabilizing mutations:
a) a disulfide bond formed between: i) a cysteine residue comprised by an amino acid extension at the C-terminus of the Cβ domain of the TCR beta chain polypeptide, where the amino acid extension has the sequence: EPKSC [SEQ ID NO:19] or EPKSCDKTHT [SEQ ID NO:16], and ii) the naturally occurring cysteine residue at position TRAC 128 in the Cα domain of the TCR alpha chain polypeptide;
b) a disulfide bond between cysteine residue substitutions at positions TRAC 84 and TRBC 79;
c) a disulfide bond between cysteine residue substitutions at positions TRAC 84.2 and TRBC 79;
d) a disulfide bond between cysteine residue substitutions at positions TRAC 122 and TRBC 12;
e) a disulfide bond between cysteine residue substitutions at positions TRAC 124 and TRBC 11;
f) a disulfide bond between cysteine residue substitutions at positions TRAC 125 and TRBC 11;
g) a disulfide bond between cysteine residue substitutions at positions TRAC 126 and TRBC 11;
h) a disulfide bond between cysteine residue substitutions at positions TRAC 127 and TRBC 11;
i) a disulfide bond between cysteine residue substitutions at positions TRAC 128 and TRBC 11;
j) a disulfide bond between cysteine residue substitutions at positions TRAC 39 and TRAC 85;
k) a disulfide bond between cysteine residue substitutions at positions TRAC 26 and TRAC 85.1;
l) an amino acid substitution at position TRAC 4 from Val to Ile;
m) an amino acid substitution at position TRAC 26 from Thr to Ile;
n) an amino acid substitution at position TRAC 39 from Val to Ile;
o) an amino acid substitution at position TRAC 85 from Ala to Val;
p) an amino acid substitution at position TRAC 105 from Ala to Ser;
q) an amino acid substitution at position TRAC 120 from Phe to Tyr;
r) an amino acid substitution at position TRBC 6 from Val to Ile or Leu;
s) an amino acid substitution at position TRBC 36 from His to Phe;
t) an amino acid substitution at position TRBC 86 from Ser to Thr;
u) an amino acid substitution at position TRBC 45.3 from Val to Thr;
v) a deletion of the amino acids at positions TRBC 84.5 to 85.6, and
w) a replacement of the amino acids at positions TRBC 84.4 to 85.4 with the amino acids Gly-Asn.

In certain embodiments, the TCR constructs comprise a combination of a first non-naturally occurring interchain disulfide bond and one or more additional stabilizing mutations, where the additional stabilizing mutations may be additional non-naturally occurring interchain disulfide bonds, non-naturally occurring intrachain disulfide bonds, point mutations, loop truncation mutations, or combinations thereof, as described herein.

In some embodiments in which the TCR construct comprises a first non-naturally occurring interchain disulfide bond and one or more additional stabilizing mutations, the first non-naturally occurring interchain disulfide bond may be a known stabilizing interchain disulfide bond, or it may be one of the stabilizing interchain disulfide bonds described herein. Examples of known stabilizing interchain disulfide bonds include, but are not limited to, the interchain disulfide bonds listed in Table 1.

TABLE 1
TCR Stability Enhancing Interchain Disulfide Bonds

| Position on Alpha Chain | Position on Beta Chain | Reference |
|---|---|---|
| TRAC 84 Thr -> Cys | TRBC 79 Ser -> Cys | Boulter, et al., 2003, |
| | | PEDS, 16:707-711 |
| TRAC 48 Thr -> Cys | TRBC 57 Ser -> Cys | WO 2004/074322¹ |
| TRAC 45 Thr -> Cys | TRBC 77 Ser -> Cys | WO 2004/074322¹ |
| TRAC 10 Tyr -> Cys | TRBC 17 Ser -> Cys | WO 2004/074322¹ |
| TRAC 45 Thr -> Cys | TRBC 59 Asp -> Cys | WO 2004/074322¹ |
| TRAC 15 Ser -> Cys | TRBC 15 Glu -> Cys | WO 2004/074322¹ |
| TRAC 53 Arg -> Cys | TRBC 54 Ser -> Cys | WO 2016/070814¹ |
| TRAC 89 Pro -> Cys | TRBC 19 Ala -> Cys | WO 2016/070814¹ |
| TRAC 10 Tyr -> Cys | TRBC 20 Glu -> Cys | WO 2016/070814¹ |
| TRAV 46 | TRBC 82 Pro -> Cys | WO 2016/184258 |
| TRAV 47 | TRBC 83 Gln -> Cys | WO 2016/184258 |
| TRAV 46 | TRBC 83 Gln -> Cys | WO 2016/184258 |
| TRAV 47 | TRBC 82 Pro -> Cys | WO 2016/184258 |
| TRAC 84.5 ARG -> Cys | TRBC 45.5 Ser -> Cys | WO 2016/070814¹ |
| TRAC 122 PRO -> Cys | TRBC 12 Ala -> Cys | WO 2016/070814¹ |
| TRAC 5 Tyr -> Cys | TRBC 13 Glu -> Cys | WO 2016/070814¹ |
| TRAV 49 | TRBV 120 | Reiter et al., 1995, |
| | | Immunity, 2(3):281-7 |
| TRAV 120 | TRBV 49 | Reiter et al., 1995, |
| | | Immunity, 2(3):281-7 |

¹TRAC and TRBC numbering used in this reference is as used in Garboczi et al, 1996, Nature, 384(6605): 134-141

In certain embodiments, the first non-naturally occurring interchain disulfide bond comprised by the TCR construct is the IC Disulfide (i.e. a disulfide bond between cysteine residue substitutions at position TRAC 84 in the TCR alpha chain polypeptide and position TRBC 79 in the TCR beta chain polypeptide).

In certain embodiments, the first non-naturally occurring interchain disulfide bond comprised by the TCR construct is selected from:
a) a disulfide bond formed between: i) a cysteine residue comprised by an amino acid extension at the C-terminus of the Cβ domain of the TCR beta chain polypeptide, where the amino acid extension is between 1 and about 10 amino acids in length, and ii) the naturally occurring cysteine residue at position TRAC 128 in the Cα domain of the TCR alpha chain polypeptide;
b) a disulfide bond between cysteine residue substitutions at positions TRAC 84.2 and TRBC 79;
c) a disulfide bond between cysteine residue substitutions at positions TRAC 122 and TRBC 12;
d) a disulfide bond between cysteine residue substitutions at positions TRAC 124 and TRBC 11;
e) a disulfide bond between cysteine residue substitutions at positions TRAC 125 and TRBC 11;
f) a disulfide bond between cysteine residue substitutions at positions TRAC 126 and TRBC 11;
g) a disulfide bond between cysteine residue substitutions at positions TRAC 127 and TRBC 11, and
h) a disulfide bond between cysteine residue substitutions at positions TRAC 128 and TRBC 11.

In some embodiments, the amino acid extension at the C-terminus of the Cβ domain of the TCR beta chain polypeptide comprises all or a portion of a hinge region sequence from an immunoglobulin or from a TCR.

In some embodiments, the first non-naturally occurring interchain disulfide bond comprised by the TCR construct is selected from:
a) a disulfide bond formed between: i) a cysteine residue in an amino acid extension at the C-terminus of the Cβ domain of the TCR beta chain polypeptide, where the amino acid extension has the sequence: EPKSC [SEQ ID NO:19] or EPKSCDKTHT [SEQ ID NO:16], and ii) the naturally occurring cysteine residue at position TRAC 128 in the Cα domain of the TCR alpha chain polypeptide;
b) a disulfide bond between cysteine residue substitutions at positions TRAC 84.2 and TRBC 79;
c) a disulfide bond between cysteine residue substitutions at positions TRAC 122 and TRBC 12;
d) a disulfide bond between cysteine residue substitutions at positions TRAC 124 and TRBC 11;
e) a disulfide bond between cysteine residue substitutions at positions TRAC 125 and TRBC 11;
f) a disulfide bond between cysteine residue substitutions at positions TRAC 126 and TRBC 11;
g) a disulfide bond between cysteine residue substitutions at positions TRAC 127 and TRBC 11, and
h) a disulfide bond between cysteine residue substitutions at positions TRAC 128 and TRBC 11.

In certain embodiments, the first non-naturally occurring interchain disulfide bond comprised by the TCR construct is selected from:
a) a disulfide bond formed between: i) a cysteine residue comprised by an amino acid extension at the C-terminus of the Cβ domain of the TCR beta chain polypeptide, where the amino acid extension is between 1 and about 10 amino acids in length, and ii) the naturally occurring cysteine residue at position TRAC 128 in the Cα domain of the TCR alpha chain polypeptide, and
b) a disulfide bond between cysteine residue substitutions at positions TRAC 84.2 and TRBC 79.

In some embodiments, the amino acid extension at the C-terminus of the Cβ domain of the TCR beta chain polypeptide comprises all or a portion of a hinge region sequence from an immunoglobulin or from a TCR.

In some embodiments, the first non-naturally occurring interchain disulfide bond comprised by the TCR construct is selected from:
a) a disulfide bond formed between: i) a cysteine residue in an amino acid extension at the C-terminus of the Cβ domain of the TCR beta chain polypeptide, where the amino acid extension has the sequence: EPKSC [SEQ ID NO:19] or EPKSCDKTHT [SEQ ID NO:16], and ii) the naturally occurring cysteine residue at position TRAC 128 in the Cα domain of the TCR alpha chain polypeptide, and
b) a disulfide bond between cysteine residue substitutions at positions TRAC 84.2 and TRBC 79.

In certain embodiments, the first non-naturally occurring interchain disulfide bond comprised by the TCR construct is selected from:
a) a disulfide bond formed between: i) a cysteine residue comprised by an amino acid extension at the C-terminus of the Cβ domain of the TCR beta chain polypeptide, where the amino acid extension is between 1 and about 10 amino acids in length, and ii) the naturally occurring cysteine residue at position TRAC 128 in the Cα domain of the TCR alpha chain polypeptide; b) a disulfide bond between cysteine residue substitutions at positions TRAC 84.2 and TRBC 79, and
c) a disulfide bond between cysteine residue substitutions at positions TRAC 84 and TRBC 79 (the IC Disulfide).

In some embodiments, the amino acid extension at the C-terminus of the Cβ domain of the TCR beta chain polypeptide comprises all or a portion of a hinge region sequence from an immunoglobulin or from a TCR.

In certain embodiments, the first non-naturally occurring interchain disulfide bond comprised by the TCR construct is selected from:
a) a disulfide bond formed between: i) a cysteine residue in an amino acid extension at the C-terminus of the Cβ domain of the TCR beta chain polypeptide, where the amino acid extension has the sequence: EPKSC [SEQ ID NO:19] or EPKSCDKTHT [SEQ ID NO:16], and ii) the naturally occurring cysteine residue at position TRAC 128 in the Cα domain of the TCR alpha chain polypeptide;
b) a disulfide bond between cysteine residue substitutions at positions TRAC 84.2 and TRBC 79, and
c) a disulfide bond between cysteine residue substitutions at positions TRAC 84 and TRBC 79 (the IC Disulfide).

In certain embodiments, the one or more additional stabilizing mutations combined with the first non-naturally occurring interchain disulfide bond are selected from the following, where the first non-naturally occurring interchain disulfide bond and any additional interchain disulfide bond are different:
a) a disulfide bond formed between: i) a cysteine residue comprised by an amino acid extension at the C-terminus of the Cβ domain of the TCR beta chain polypeptide, where the amino acid extension is between 1 and about 10 amino acids in length, and ii) the naturally occurring cysteine residue at position TRAC 128 in the Cα domain of the TCR alpha chain polypeptide;
b) a disulfide bond between cysteine residue substitutions at positions TRAC 84.2 and TRBC 79;
c) a disulfide bond between cysteine residue substitutions at positions TRAC 122 and TRBC 12;
d) a disulfide bond between cysteine residue substitutions at positions TRAC 124 and TRBC 11;
e) a disulfide bond between cysteine residue substitutions at positions TRAC 125 and TRBC 11;
f) a disulfide bond between cysteine residue substitutions at positions TRAC 126 and TRBC 11;
g) a disulfide bond between cysteine residue substitutions at positions TRAC 127 and TRBC 11;
h) a disulfide bond between cysteine residue substitutions at positions TRAC 128 and TRBC 11;
i) a disulfide bond between cysteine residue substitutions at positions TRAC 39 and TRAC 85;
j) a disulfide bond between cysteine residue substitutions at positions TRAC 26 and TRAC 85.1;
k) an amino acid substitution at position TRAC 4 from Val to Ala, Thr, Ile, Leu or Met;
l) an amino acid substitution at position TRAC 26 from Thr to Ala, Val, Ile, Leu or Met;
m) an amino acid substitution at position TRAC 39 from Val to Ala, Thr, Ile, Leu or Met;
n) an amino acid substitution at position TRAC 85 from Ala to Ser, Thr, Val, Ile or Met;
o) an amino acid substitution at position TRAC 105 from Ala to Ser, Thr, Glu, Gln, Asp, Asn, His, Lys or Arg;
p) an amino acid substitution at position TRAC 120 from Phe to Tyr or His;
q) an amino acid substitution at position TRBC 6 from Val to Ala, Thr, Ile, Leu or Met;
r) an amino acid substitution at position TRBC 36 from His to Phe, Tyr or Trp;
s) an amino acid substitution at position TRBC 86 from Ser to Ala or Thr;
t) an amino acid substitution at position TRBC 45.3 from Val to Ser, Thr, Glu, Gln, Asp, Asn, His, Lys or Arg;
u) a deletion of between 1 and 4 consecutive amino acids of the DE loop in the Cβ domain of the TCR construct, and
v) a replacement of the amino acids at positions TRBC 84.4 to 85.4 with an amino acid sequence of between 2 and 4 amino acids that allows for formation of a beta-turn.

In some embodiments, the amino acid extension at the C-terminus of the Cβ domain of the TCR beta chain polypeptide comprises all or a portion of a hinge region sequence from an immunoglobulin or from a TCR. In some embodiments, the amino acid extension at the C-terminus of the Cβ domain of the TCR beta chain polypeptide has the sequence: EPKSC [SEQ ID NO:19] or EPKSCDKTHT [SEQ ID NO:16].

In some embodiments, the amino acid substitution at position TRAC 4 is from Val to Ile. In some embodiments, the amino acid substitution at position TRAC 26 is from Thr to Ile. In some embodiments, the amino acid substitution at position TRAC 39 is from Val to Ile. In some embodiments, the amino acid substitution at position TRAC 85 is from Ala to Val. In some embodiments, the amino acid substitution at position TRAC 105 is from Ala to Ser. In some embodiments, the amino acid substitution at position TRAC 120 is from Phe to Tyr. In some embodiments, the amino acid substitution at position TRBC 6 is from Val to Leu or Ile. In some embodiments, the amino acid substitution at position TRBC 36 is from His to Phe. In some embodiments, the amino acid substitution at position TRBC 86 is from Ser to Thr. In some embodiments, the amino acid substitution at position TRBC 45.3 is from Val to Thr.

In some embodiments, the deletion of between 1 and 4 consecutive amino acids of the DE loop in the Cβ domain of the TCR construct is a deletion of the amino acids at positions TRBC 84.5 to 85.6.

In some embodiments, the replacement of the amino acids at positions TRBC 84.4 to 85.4 is with the amino acids Gly-Asn.

Combinations of any of the foregoing embodiments for TCR constructs are also contemplated and each combination forms a separate embodiment for the purposes of the present disclosure.

In some embodiments, the one or more additional stabilizing mutations combined with the first non-naturally occurring interchain disulfide bond are selected from the following, where the first non-naturally occurring interchain disulfide bond and any additional interchain disulfide bond are different:
a) a disulfide bond formed between: i) a cysteine residue in an amino acid extension at the C-terminus of the Cβ domain of the TCR beta chain polypeptide, where the amino acid extension has the sequence: EPKSC [SEQ ID NO:19] or EPKSCDKTHT [SEQ ID NO:16], and ii) the naturally occurring cysteine residue at position TRAC 128 in the Cα domain of the TCR alpha chain polypeptide;
b) a disulfide bond between cysteine residue substitutions at positions TRAC 84.2 and TRBC 79;
c) a disulfide bond between cysteine residue substitutions at positions TRAC 122 and TRBC 12;
d) a disulfide bond between cysteine residue substitutions at positions TRAC 124 and TRBC 11;
e) a disulfide bond between cysteine residue substitutions at positions TRAC 125 and TRBC 11;
f) a disulfide bond between cysteine residue substitutions at positions TRAC 126 and TRBC 11;
g) a disulfide bond between cysteine residue substitutions at positions TRAC 127 and TRBC 11;
h) a disulfide bond between cysteine residue substitutions at positions TRAC 128 and TRBC 11;
i) a disulfide bond between cysteine residue substitutions at positions TRAC 39 and TRAC 85;
j) a disulfide bond between cysteine residue substitutions at positions TRAC 26 and TRAC 85.1;
k) an amino acid substitution at position TRAC 4 from Val to Ile;
l) an amino acid substitution at position TRAC 26 from Thr to Ile;
m) an amino acid substitution at position TRAC 39 from Val to Ile;
n) an amino acid substitution at position TRAC 85 from Ala to Val;
o) an amino acid substitution at position TRAC 105 from Ala to Ser;
p) an amino acid substitution at position TRAC 120 from Phe to Tyr;
q) an amino acid substitution at position TRBC 6 from Val to Ile or Leu;
r) an amino acid substitution at position TRBC 36 from His to Phe;
s) an amino acid substitution at position TRBC 86 from Ser to Thr;
t) an amino acid substitution at position TRBC 45.3 from Val to Thr;
u) a deletion of the amino acids at positions TRBC 84.5 to 85.6, and
v) a replacement of the amino acids at positions TRBC 84.4 to 85.4 with the amino acids Gly-Asn.

In certain embodiments, the one or more additional stabilizing mutations combined with a non-naturally occurring interchain disulfide bond are selected from the following:
a) an amino acid substitution at position TRAC 39 from Val to Ala, Thr, Ile, Leu or Met;
b) an amino acid substitution at position TRAC 85 from Ala to Ser, Thr, Val, Ile or Met;
c) an amino acid substitution at position TRBC 6 from Val to Ala, Thr, Ile, Leu or Met; and
d) an amino acid substitution at position TRBC 36 from His to Phe, Tyr or Trp.

In certain embodiments, the one or more additional stabilizing mutations combined with a non-naturally occurring interchain disulfide bond are selected from the following:
a) an amino acid substitution at position TRAC 39 from Val to Ile;
b) an amino acid substitution at position TRAC 85 from Ala to Val;
c) an amino acid substitution at position TRBC 6 from Val to Ile, and
d) an amino acid substitution at position TRBC 36 from His to Phe.

In certain embodiments, the additional stabilizing mutations combined with a non-naturally occurring interchain disulfide bond are the following:
a) an amino acid substitution at position TRAC 39 from Val to Ile;
b) an amino acid substitution at position TRAC 85 from Ala to Val;
c) an amino acid substitution at position TRBC 6 from Val to Ile, and
d) an amino acid substitution at position TRBC 36 from His to Phe.

In certain embodiments, the TCR constructs comprise a first non-naturally occurring interchain disulfide bond and one or more additional stabilizing mutations, where the first non-naturally occurring disulfide bond is a disulfide bond formed between: i) a cysteine residue comprised by an amino acid extension at the C-terminus of the Cβ domain of the TCR beta chain polypeptide, where the amino acid extension is between 1 and about 10 amino acids in length, and ii) the naturally occurring cysteine residue at position TRAC 128 in the Cα domain of the TCR alpha chain polypeptide, and the one or more stabilizing mutations comprise an additional non-naturally occurring interchain disulfide bond selected from:
a) a disulfide bond between cysteine residue substitutions at positions TRAC 84 and TRBC 79;
b) a disulfide bond between cysteine residue substitutions at positions TRAC 84.2 and TRBC 79;
c) a disulfide bond between cysteine residue substitutions at positions TRAC 122 and TRBC 12;
d) a disulfide bond between cysteine residue substitutions at positions TRAC 124 and TRBC 11;
e) a disulfide bond between cysteine residue substitutions at positions TRAC 125 and TRBC 11;
f) a disulfide bond between cysteine residue substitutions at positions TRAC 126 and TRBC 11;
g) a disulfide bond between cysteine residue substitutions at positions TRAC 127 and TRBC 11, and
h) a disulfide bond between cysteine residue substitutions at positions TRAC 128 and TRBC 11.

In some embodiments, the amino acid extension at the C-terminus of the Cβ domain of the TCR beta chain polypeptide comprises all or a portion of a hinge region sequence from an immunoglobulin or from a TCR.

In some embodiments, the first non-naturally occurring interchain disulfide bond comprised by the TCR construct is a disulfide bond formed between: i) a cysteine residue in an amino acid extension at the C-terminus of the Cβ domain of the TCR beta chain polypeptide, where the amino acid extension has the sequence: EPKSC [SEQ ID NO:19] or EPKSCDKTHT [SEQ ID NO:16], and ii) the naturally occurring cysteine residue at position TRAC 128 in the Cα domain of the TCR alpha chain polypeptide.

In certain embodiments, the TCR constructs comprise a first non-naturally occurring interchain disulfide bond and one or more additional stabilizing mutations, where the first non-naturally occurring disulfide bond is a disulfide bond between cysteine residue substitutions at positions TRAC 84.2 and TRBC 79, and the one or more stabilizing mutations comprise an additional non-naturally occurring interchain disulfide bond selected from:
a) a disulfide bond formed between: i) a cysteine residue comprised by an amino acid extension at the C-terminus of the Cβ domain of the TCR beta chain polypeptide, where the amino acid extension is between 1 and about 10 amino acids in length, and ii) the naturally occurring cysteine residue at position TRAC 128 in the Cα domain of the TCR alpha chain polypeptide;
b) a disulfide bond between cysteine residue substitutions at positions TRAC 122 and TRBC 12;
c) a disulfide bond between cysteine residue substitutions at positions TRAC 124 and TRBC 11;
d) a disulfide bond between cysteine residue substitutions at positions TRAC 125 and TRBC 11;
e) a disulfide bond between cysteine residue substitutions at positions TRAC 126 and TRBC 11;
f) a disulfide bond between cysteine residue substitutions at positions TRAC 127 and TRBC 11, and
g) a disulfide bond between cysteine residue substitutions at positions TRAC 128 and TRBC 11.

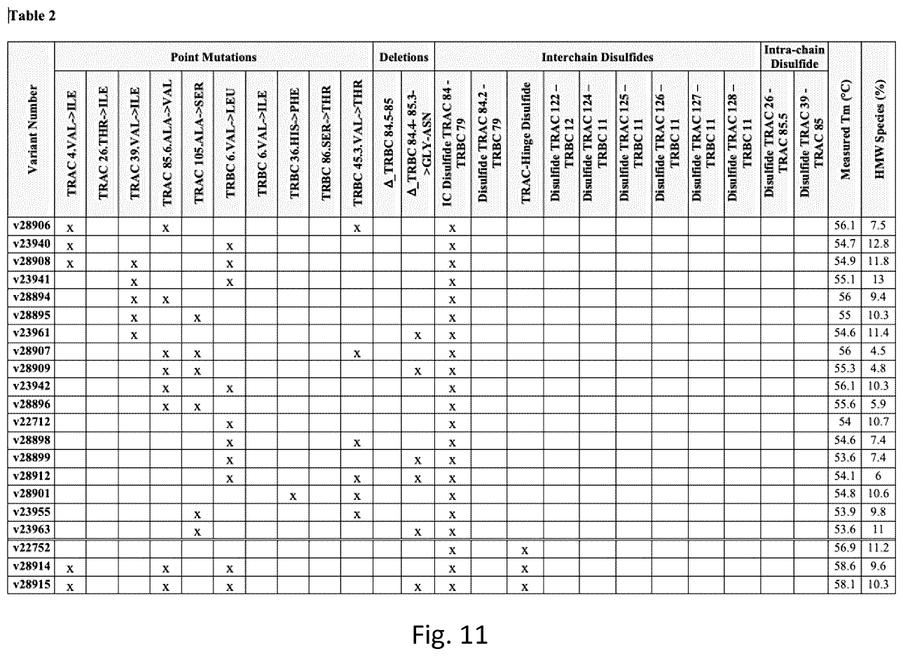

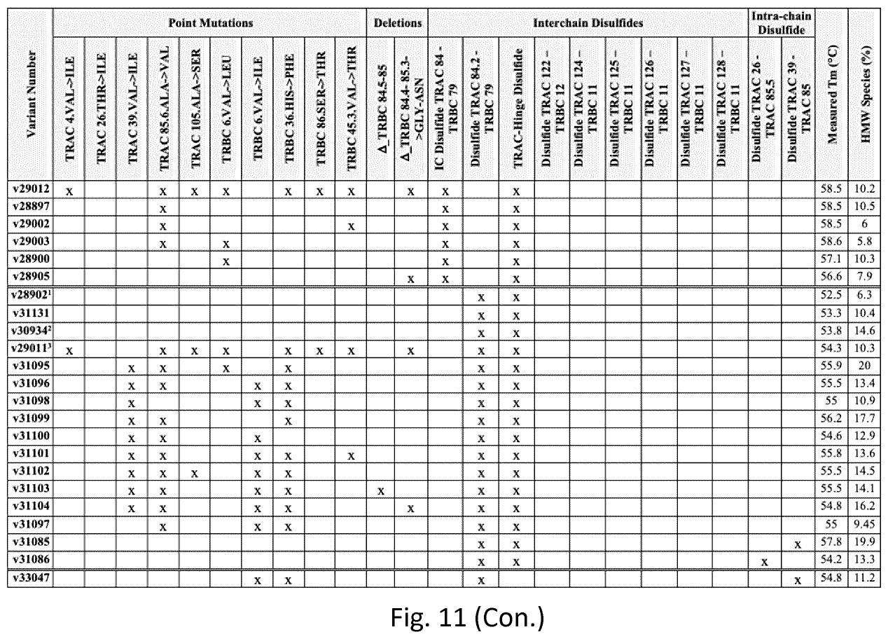

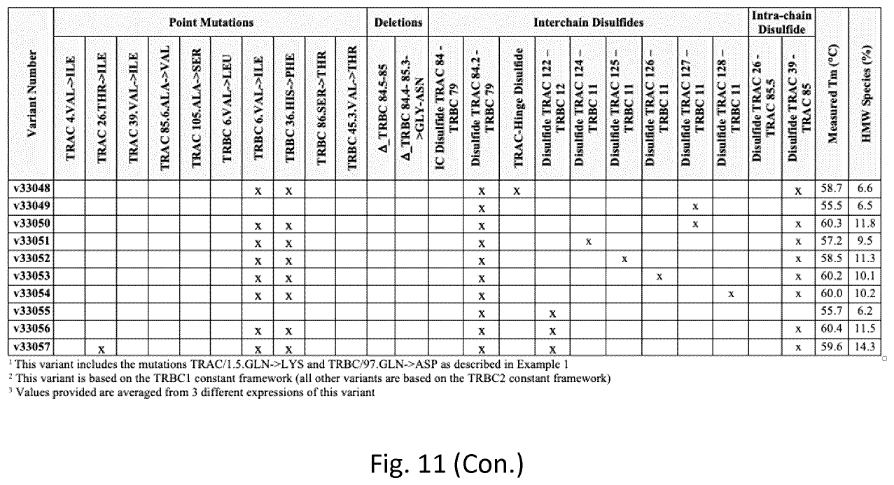

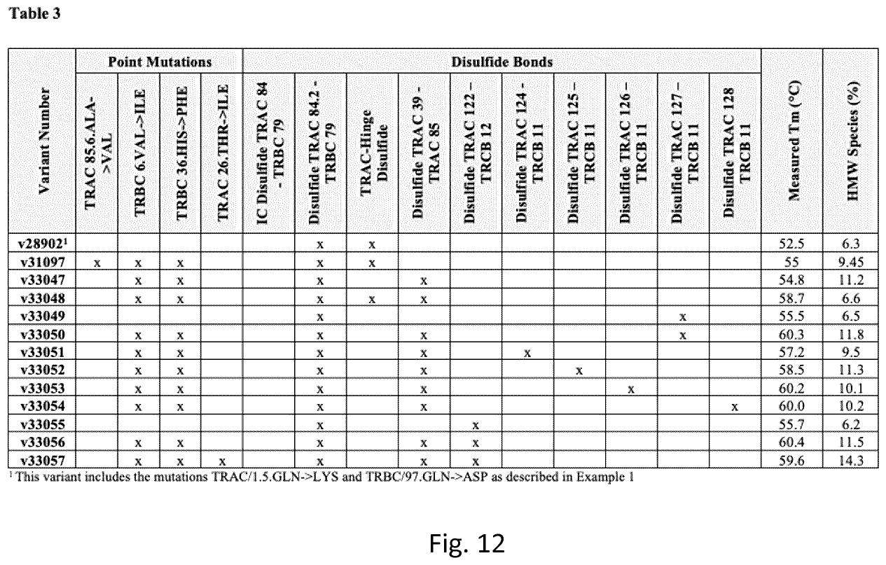

In certain embodiments, the TCR constructs of the present disclosure comprise a combination of a first non-naturally occurring interchain disulfide bond and one or more additional stabilizing mutations, where the combination is one of the combinations as set forth for any one of the variants listed in Table 2 (FIG. 11). In some embodiments, the TCR constructs of the present disclosure comprise a combination of a first non-naturally occurring interchain disulfide bond and one or more additional stabilizing mutations, where the combination is one of the combinations as set forth for any one of the variants listed in Table 3 (FIG. 12).

In certain embodiments, the TCR constructs of the present disclosure comprise a combination of two or more stabilizing mutations where the stabilizing mutations are point mutations and/or loop truncation mutations. In some embodiments, the TCR constructs comprise two or more stabilizing mutations selected from:
i) an amino acid substitution at position TRAC 4 from Val to Ala, Thr, Ile, Leu or Met;
ii) an amino acid substitution at position TRAC 26 from Thr to Ala, Val, Ile, Leu or Met;
iii) an amino acid substitution at position TRAC 39 from Val to Ala, Thr, Ile, Leu or Met;
iv) an amino acid substitution at position TRAC 85 from Ala to Ser, Thr, Val, Ile or Met;
v) an amino acid substitution at position TRAC 105 from Ala to Ser, Thr, Glu, Gln, Asp, Asn, His, Lys or Arg;
vi) an amino acid substitution at position TRAC 120 from Phe to Tyr or His;
vii) an amino acid substitution at position TRBC 6 from Val to Ala, Thr, Ile, Leu or Met;
viii) an amino acid substitution at position TRBC 36 from His to Phe, Tyr or Trp;
ix) an amino acid substitution at position TRBC 86 from Ser to Ala or Thr;
x) an amino acid substitution at position TRBC 45.3 from Val to Ser, Thr, Glu, Gln, Asp, Asn, His, Lys or Arg;
xi) a deletion of between 1 and 4 consecutive amino acids of the DE loop in the Cβ domain of the TCR construct, and
xii) a replacement of the amino acids at positions TRBC 84.4 to 85.4 with an amino acid sequence of between 2 and 4 amino acids that allows for formation of a beta-turn.

In some embodiments, the TCR constructs comprise two or more stabilizing mutations selected from:
i) an amino acid substitution at position TRAC 4 from Val to Ile;
ii) an amino acid substitution at position TRAC 26 from Thr to Ile;
iii) an amino acid substitution at position TRAC 39 from Val to Ile;
iv) an amino acid substitution at position TRAC 85 from Ala to Val;
v) an amino acid substitution at position TRAC 105 from Ala to Ser;
vi) an amino acid substitution at position TRAC 120 from Phe to Tyr;
vii) an amino acid substitution at position TRBC 6 from Val to Ile or Leu;
viii) an amino acid substitution at position TRBC 36 from His to Phe;
ix) an amino acid substitution at position TRBC 86 from Ser to Thr;
x) an amino acid substitution at position TRBC 45.3 from Val to Thr;
xi) a deletion of the amino acids at positions TRBC 84.5 to 85.6, and
xii) a replacement of the amino acids at positions TRBC 84.4 to 85.4 with the amino acids Gly-Asn.

### TCR Fusion Proteins

Certain embodiments of the present disclosure relate to TCR fusion proteins comprising one or more TCR constructs as described herein operably linked to a scaffold and/or to one or more additional biologically active moieties. The term “operably linked,” as used herein, means that the components described are in a relationship permitting each of them to function in their intended manner. A TCR construct may be directly or indirectly linked to the scaffold or additional biologically active moiety in the TCR fusion protein. By indirectly linked, it is meant that a given TCR construct is linked to the scaffold or biologically active moiety via another component, for example, a linker or a second TCR construct. Various formats are contemplated for TCR fusion proteins as described in more detail below.

TCR fusion proteins of the present disclosure may comprise one TCR construct or they may comprise multiple TCR constructs. The number of TCR constructs that may be comprised by a TCR fusion protein will depend on the nature of the scaffold and/or additional biologically active moieties comprised by the fusion protein. Typically, a TCR fusion protein of the present disclosure comprises between 1 and 24 TCR constructs. In certain embodiments, a TCR fusion protein may comprise between 1 and 12 TCR constructs, between 1 and 8 TCR constructs, between 1 and 6 TCR constructs, between 1 and 4 TCR constructs or between 1 and 3 TCR constructs.

### Scaffolds

In certain embodiments, the TCR fusion proteins of the present disclosure comprise a scaffold. The scaffold may be, for example, a protein (including a peptide or polypeptide), a polymer, a nanoparticle or another chemical entity. Where the scaffold is a protein, each TCR construct comprised by the TCR fusion protein is typically linked to either the N- or C-terminus of the protein scaffold, although linkage to a region other than the N- or C-terminus, for example, via the side chain of an amino acid with or without a linker, is also contemplated in certain embodiments.

In embodiments where the scaffold is a protein, a TCR construct may be linked to the scaffold by genetic fusion or chemical conjugation. In embodiments where the scaffold is a polymer or nanoparticle, a TCR construct is typically linked to the scaffold by chemical conjugation.

In certain embodiments, the TCR fusion protein comprises a protein scaffold. Examples of protein scaffolds include immunoglobulin (Ig) Fc regions, albumin, albumin analogues and derivatives, toxins, cytokines, chemokines, growth factors and protein pairs such as leucine zipper domains (for example, Fos and Jun) (Kostelny, et al., 1992, J Immunol, 148:1547-53; Wranik, et al., 2012, J. Biol. Chem., 287: 43331-43339), the barnase barstar pair (Deyev, et al., 2003, Nat Biotechnol, 21:1486-1492) and split fluorescent protein pairs (International Publication No. WO 2011/13504). Examples of albumin derivatives include those described in International Publication Nos. WO 2012/116453 and WO 2014/012082, which may be fused to up to four different TCR constructs, optionally via linkers.

Other protein scaffolds that have been described in combination with various binding domains are also contemplates in certain embodiments (for example, see Müller et al., 2007, J Biol Chem, 282:12650-12660; McDonaugh et al., 2012, Mol Cancer Ther, 11:582-593; Vallera et al., 2005, Clin Cancer Res, 11:3879-3888; Song et al., 2006, Biotech Appl Biochem, 45:147-154; U.S. Patent Application Publication No. US2009/0285816 and International Publication Nos. WO 2012/116453 and WO 2014/012082).

### Fc Regions

In certain embodiments, the TCR fusion proteins described herein comprise a scaffold that is based on an immunoglobulin (Ig) Fc region. The terms “Fc region,” “Fc” and “Fc domain,” as used interchangeably herein, refer to a C-terminal region of an immunoglobulin heavy chain that contains at least a portion of the constant region. The term includes native sequence Fc regions and variant Fc regions. An “Fc polypeptide” of a dimeric Fc refers to one of the two polypeptides forming the dimeric Fc region, that is a polypeptide comprising C-terminal constant regions of an immunoglobulin heavy chain that is capable of stable self-association.

Unless otherwise specified herein, numbering of amino acid residues in the Fc region or constant region of an Ig is according to the EU numbering system, also called the EU index, as described in Kabat et al, Sequences of Proteins of Immunological Interest, 5th Ed. Public Health Service, National Institutes of Health, Bethesda, MD (1991).

An Fc region may comprise just a CH3 domain, or both a CH3 and a CH2 domain. The CH3 domain comprises two CH3 sequences, each comprised by one of the two Fc polypeptides of the dimeric Fc. Similarly, the CH2 domain comprises two CH2 sequences, each comprised by one of the two Fc polypeptides of the dimeric Fc.

An Ig Fc region comprised by a TCR fusion protein may be based on an IgG, IgA or IgM Fc region. In certain embodiments, the TCR fusion protein comprises a scaffold based on an IgG Fc. In some embodiments, the TCR fusion protein comprises a scaffold based on an IgG1 Fc. In some embodiments, the TCR fusion protein comprises a scaffold based on a human IgG Fc. In some embodiments, the TCR fusion protein comprises a scaffold based on a human IgG1 Fc.

In certain embodiments, the Ig Fc region comprised by the TCR fusion protein may be a variant Fc region that comprises one or more amino acid modifications in the CH3 domain, the CH2 domain or both. In some embodiments, the Ig Fc region comprised by the TCR fusion protein may be a variant Fc region that is a heterodimeric Fc comprising two different Fc polypeptides.

In certain embodiments, the TCR fusion protein comprises a scaffold based on a variant IgG Fc in which the CH3 domain comprises one or more amino acid modifications (a “modified CH3 domain”). In some embodiments, the TCR fusion protein comprises a scaffold based on a variant IgG Fc in which the CH2 domain comprises one or more amino acid modifications (a “modified CH2 domain”). In some embodiments, the TCR fusion protein comprises a scaffold based on a variant IgG Fc in which the CH3 domain comprises one or more amino acid modifications and the CH2 domain comprises one or more amino acid modifications.

### Modified CH3 Domains

In certain embodiments, the TCR fusion protein described herein comprises a heterodimeric Ig Fc comprising a modified CH3 domain. In some such embodiments, the modified CH3 domain comprises one or more asymmetric amino acid modifications. As used herein, an “asymmetric amino acid modification” refers to a modification in which an amino acid at a specific position on the first Fc polypeptide is different to the amino acid at the corresponding position on the second Fc polypeptide. These asymmetric amino acid modifications may comprise modification of only one of the two amino acids at the corresponding position on each Fc polypeptide, or they may comprise modifications of both amino acids at the corresponding positions on each of the first and second Fc polypeptides.

In certain embodiments, the TCR fusion protein comprises a heterodimeric Fc comprising a modified CH3 domain, where the modified CH3 domain comprises one or more asymmetric amino acid modifications that promote formation of the heterodimeric Fc over formation of a homodimeric Fc. Amino acid modifications that may be made to the CH3 domain of an Fc in order to promote formation of a heterodimeric Fc are known in the art and include, for example, those described in International Publication No. WO 96/027011 (“knobs into holes”), Gunasekaran et al., 2010, J Biol Chem, 285, 19637-46 (“electrostatic steering”), Davis et al., 2010, Prot Eng Des Sel, 23(4):195-202 (strand exchange engineered domain (SEED) technology) and Labrijn et al., 2013, Proc Natl Acad Sci USA, 110(13):5145-50 (Fab-arm exchange). Other examples include approaches combining positive and negative design strategies to produce stable asymmetrically modified Fc regions as described in International Publication Nos. WO 2012/058768 and WO 2013/063702.

In certain embodiments, the TCR fusion protein comprises a scaffold that is a heterodimeric Fc having a modified CH3 domain as described in International Publication No. WO 2012/058768 or International Patent Publication No. WO 2013/063702.

In some embodiments, the TCR fusion protein comprises a scaffold that is a heterodimeric human IgG1 Fc having a modified CH3 domain. Table 4 below provides the amino acid sequence of the human IgG1 Fc sequence, corresponding to amino acids 231 to 447 of the full-length human IgG1 heavy chain. The CH2 domain is typically defined as comprising amino acids 231-340 of the full-length human IgG1 heavy chain and the CH3 domain is typically defined as comprising amino acids 341-447 of the full-length human IgG1 heavy chain.

In certain embodiments, the TCR fusion protein comprises a heterodimeric Fc having a modified CH3 domain comprising one or more asymmetric amino acid modifications that promote formation of the heterodimeric Fc over formation of a homodimeric Fc, in which the modified CH3 domain comprises a first Fc polypeptide including amino acid substitutions at positions F405 and Y407, and a second Fc polypeptide including amino acid substitutions at positions T366 and T394. In some embodiments, the amino acid substitution at position F405 of the first Fc polypeptide of the modified CH3 domain is F405A, F405I, F405M, F405S, F405T or F405V. In some embodiments, the amino acid substitution at position Y407 of the first Fc polypeptide of the modified CH3 domain is Y407I or Y407V. In some embodiments, the amino acid substitution at position T366 of the second Fc polypeptide of the modified CH3 domain is T366I, T366L or T366M. In some embodiments, the amino acid substitution at position T394 of the second Fc polypeptide of the modified CH3 domain is T394W. In some embodiments, the first Fc polypeptide of the modified CH3 domain further includes an amino acid substitution at position L351 which is L351Y. In some embodiments, the second Fc polypeptide of the modified CH3 domain further includes an amino acid substitution at position K392 selected from K392F, K392L and K392M. In some embodiments, one or both of the first and second Fc polypeptides of the modified CH3 domain further comprises the amino acid substitution T350V.

In certain embodiments, the TCR fusion protein comprises a heterodimeric Fc having a modified CH3 domain comprising one or more asymmetric amino acid modifications that promote formation of the heterodimeric Fc over formation of a homodimeric Fc, in which the modified CH3 domain comprises a first Fc polypeptide including the amino acid substitution F405A, F405I, F405M, F405S, F405T or F405V together with the amino acid substitution Y407I or Y407V, and a second Fc polypeptide including the amino acid substitution T366I, T366L or T366M, together with the amino acid substitution T394W. In some embodiments, the first Fc polypeptide of the modified CH3 domain further includes the amino acid substitution L351Y and/or the second Fc polypeptide of the modified CH3 domain further includes the amino acid substitution K392F, K392L or K392M. In some embodiments, one or both of the first and second Fc polypeptides of the modified CH3 domain further comprises the amino acid substitution T350V.

In certain embodiments, the TCR fusion protein comprises a heterodimeric Fc comprising a modified CH3 domain having a first Fc polypeptide that comprises amino acid substitutions at positions F405 and Y407, and optionally further comprises an amino acid substitution at position L351, and a second Fc polypeptide that comprises amino acid substitutions at positions T366 and T394, and optionally further comprises an amino acid substitution at position K392, as described above, and the first Fc polypeptide further comprises an amino acid substitution at one or both of positions S400 or Q347 and/or the second Fc polypeptide further comprises an amino acid substitution at one or both of positions K360 or N390, where the amino acid substitution at position S400 is S400E, S400D, S400R or S400K; the amino acid substitution at position Q347 is Q347R, Q347E or Q347K; the amino acid substitution at position K360 is K360D or K360E, and the amino acid substitution at position N390 is N390R, N390K or N390D.

In certain embodiments, the TCR fusion protein comprises a heterodimeric Fe comprising a modified CH3 domain comprising the amino acid substitutions of any one of Variant 1, Variant 2, Variant 3, Variant 4 or Variant 5, as shown in Table 4.

TABLE 4
Human IgG1 Fc Sequences and Variants

| Human IgG1 Fc | APELLGGPSVFLFPPKPKDTLMISRTPEVTCVVVDVSH | |
|---|---|---|
| sequence 231-447 (EU- | EDPEVKFNWYVDGVEVHNAKTKPREEQYNSTYRVVS | |
| numbering) | VLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| | (SEQ ID NO: 20) | |
| Variant # | Chain | Mutations |
| 1 | A | L351Y_F405A_Y407V |
| | B | T366L_K392M_T394W |
| 2 | A | L351Y_F405A_Y407V |
| | B | T366L_K392L_T394W |
| 3 | A | T350V_L351Y_F405A_Y407V |
| | B | T350V_T366L_K392L_T394W |
| 4 | A | T350V_L351Y_F405A_Y407V |
| | B | T350V_T366L_K392M_T394W |
| 5 | A | T350V_L351Y_S400E_F405A_Y407V |
| | B | T350V_T366L_N390R_K392M_T394W |

### Modified CH2 Domains

In certain embodiments, the TCR fusion protein comprises a scaffold based on an IgG Fc having a modified CH2 domain, for example, a CH2 domain comprising amino acid modifications that result in altered binding to one or more Fc receptors (FcRs). In some embodiments, the amino acid modifications in the CH2 domain result in altered binding to one or more of the FcγRI, FcγRII and FcγRIII subclasses of Fc receptor.

A number of amino acid modifications to the CH2 domain that selectively alter the affinity of the Fc for different Fcγ receptors are known in the art. Amino acid modifications that result in increased binding and amino acid modifications that result in decreased binding can each be useful in certain indications. For example, increasing binding affinity of an Fc for FcγRIIIa (an activating receptor) results in increased antibody dependent cell-mediated cytotoxicity (ADCC), which in turn results in increased lysis of the target cell. Decreased binding to FcγRIIb (an inhibitory receptor) likewise may be beneficial in some circumstances. In certain indications, a decrease in, or elimination of, ADCC and complement-mediated cytotoxicity (CDC) may be desirable. In such cases, modified CH2 domains comprising amino acid modifications that result in increased binding to FcγRIIb or amino acid modifications that decrease or eliminate binding of the Fc region to all of the Fcγ receptors (“knock-out” variants) may be useful.

Examples of amino acid modifications to the CH2 domain that alter binding of the Fc by Fcγ receptors include, but are not limited to, the following: S298A/E333A/K334A and S298A/E333A/K334A/K326A (increased affinity for FcγRIIIa) (Lu, et al., 2011, J Immunol Methods, 365(1-2):132-41); F243L/R292P/Y300L/V305I/P396L (increased affinity for FcγRIIIa) (Stavenhagen, et al., 2007, Cancer Res, 67(18):8882-90); F243L/R292P/Y300L/L235V/P396L (increased affinity for FcγRIIIa) (Nordstrom J L, et al., 2011, Breast Cancer Res, 13(6):R123); F243L (increased affinity for FcγRIIIa) (Stewart, et al., 2011, Protein Eng Des Sel., 24(9):671-8); S298A/E333A/K334A (increased affinity for FcγRIIIa) (Shields, et al., 2001, J Biol Chem, 276(9):6591-604); S239D/I332E/A330L and S239D/I332E (increased affinity for FcγRIIIa) (Lazar, et al., 2006, Proc Natl Acad Sci USA, 103(11):4005-10), and S239D/S267E and S267E/L328F (increased affinity for FcγRIIb) (Chu, et al., 2008, Mol Immunol, 45(15):3926-33).

Additional modifications that affect Fc binding to Fcγ receptors are described in Therapeutic Antibody Engineering (Strohl & Strohl, Woodhead Publishing series in Biomedicine No 11, ISBN 1 907568 37 9, October 2012, page 283).

In certain embodiments, the TCR fusion protein comprises a scaffold based on an IgG Fc having a modified CH2 domain, in which the modified CH2 domain comprises one or more amino acid modifications that result in decreased or eliminated binding of the Fc region to all Fcγ receptors (i.e. a “knock-out” variant).

Various publications describe strategies that have been used to engineer Fc regions to produce “knock-out” variants (see, for example, Strohl, 2009, Curr Opin Biotech 20:685-691, and Strohl & Strohl, “Antibody Fc engineering for optimal antibody performance” In Therapeutic Antibody Engineering, Cambridge: Woodhead Publishing, 2012, pp 225-249). These strategies include reduction of effector function through modification of glycosylation (described in more detail below), use of IgG2/IgG4 scaffolds, or the introduction of mutations in the hinge or CH2 domain of the Fc (see for example, U.S. Patent Publication No. 2011/0212087, International Publication No. WO 2006/105338, U.S. Patent Publication No. 2012/0225058, U.S. Patent Publication No. 2012/0251531 and Strop et al., 2012, J Mol. Biol., 420: 204-219).

Specific, non-limiting examples of known amino acid modifications to reduce FcγR and/or complement binding to the Fc include those identified in Table 5.

TABLE 5
Modifications to Reduce Fcγ Receptor or Complement Binding to the Fc

| Company | Mutations |
|---|---|
| GSK | N297A |
| Ortho Biotech | L234A/L235A |
| Protein Design labs | IgG2 V234A/G237A |
| Wellcome Labs | IgG4 L235A/G237A/E318A |
| GSK | IgG4 S228P/L236E |
| Merck | IgG2 H268Q/V309L/A330S/A331S |
| Bristol-Myers | C220S/C226S/C229S/P238S |
| Seattle Genetics | C226S/C229S/E3233P/L235V/L235A |
| Medimmune | L234F/L235E/P331S |

Additional examples include Fc regions engineered to include the amino acid substitutions L235A/L236A/D265S and the asymmetric amino acid modifications in the CH2 domain described in International Publication No. WO 2014/190441.

### Glycosylation Variants

In certain embodiments, the TCR fusion proteins described herein may comprise a scaffold based on an IgG Fc in which native glycosylation has been modified. As is known in the art, glycosylation of an Fc may be modified to increase or decrease effector function.

For example, mutation of the conserved asparagine residue at position 297 to alanine, glutamine, lysine or histidine (i.e. N297A, N297Q, N297K or N297H) results in an aglycoslated Fc that lacks all effector function (Bolt et al., 1993, Eur. J. Immunol., 23:403-411; Tao & Morrison, 1989, J. Immunol., 143:2595-2601).

Conversely, removal of fucose from heavy chain N297-linked oligosaccharides has been shown to enhance ADCC, based on improved binding to FcγRIIIa (see, for example, Shields et al., 2002, J Biol Chem., 277:26733-26740, and Niwa et al., 2005, J. Immunol. Methods, 306:151-160). Such low fucose antibodies may be produced, for example in knockout Chinese hamster ovary (CHO) cells lacking fucosyltransferase (FUT8) (Yamane-Ohnuki et al., 2004, Biotechnol. Bioeng., 87:614-622); in the variant CHO cell line, Lec 13, that has a reduced ability to attach fucose to N297-linked carbohydrates (International Publication No. WO 2003/035835), or in other cells that generate afucosylated antibodies (see, for example, Li et al., 2006, Nat Biotechnol, 24:210-215; Shields et al., 2002, ibid, and Shinkawa et al., 2003, J. Biol. Chem., 278:3466-3473). In addition, International Publication No. WO 2009/135181 describes the addition of fucose analogs to culture medium during antibody production to inhibit incorporation of fucose into the carbohydrate on the antibody.

Other methods of producing antibodies with little or no fucose on the Fc glycosylation site (N297) are known in the art. For example, the GlymaX® technology (ProBioGen AG) (see von Horsten et al., 2010, Glycobiology, 20(12):1607-1618 and U.S. Pat. No. 8,409,572).

Other glycosylation variants include those with bisected oligosaccharides, for example, variants in which a biantennary oligosaccharide attached to the Fc region of the antibody is bisected by N-acetylglucosamine (GlcNAc). Such glycosylation variants may have reduced fucosylation and/or improved ADCC function (see, for example, International Publication No. WO 2003/011878, U.S. Pat. No. 6,602,684 and U.S. Patent Application Publication No. US 2005/0123546). Useful glycosylation variants also include those having at least one galactose residue in the oligosaccharide attached to the Fc region, which may have improved CDC function (see, for example, International Publication Nos. WO 1997/030087, WO 1998/58964 and WO 1999/22764).

### Additional Biologically Active Moieties

In certain embodiments, the TCR fusion proteins of the present disclosure may comprise one or more additional biologically active moieties fused or covalently attached to a TCR construct and/or, when the TCR fusion protein comprises a scaffold, to the scaffold. Examples of additional biologically active moieties that may be comprised by the TCR fusion protein include, but are not limited to, antigen-binding domains, ligands, receptors, receptor fragments (such as extracellular portions), cytokines and antigens. When the TCR fusion proteins comprise more than one additional biologically active moiety, the moieties may be the same or they may be different.

In certain embodiments, the TCR fusion proteins of the present disclosure comprise one or more additional biologically active moiety. In some embodiments, the TCR fusion proteins comprise between 1 and 6 additional biologically active moieties. In some embodiments, the TCR fusion proteins comprise between 1 and 4 additional biologically active moieties.

In certain embodiments, the TCR fusion proteins may comprise one or more additional biologically active moieties that are antigen-binding domains. In some embodiments, the TCR fusion proteins may comprise two or more antigen-binding domains, for example, 2, 3, 4, 5 or 6 antigen-binding domains. When the TCR fusion protein comprises two or more antigen-binding domains, the antigen-binding domains may bind the same antigen, or they may bind different antigens.

Non-limiting examples of antigen-binding domains that may be included in a TCR fusion protein in some embodiments include Fab fragments, Fv fragments, single-chain Fv fragments (scFv) and single domain antibodies (sdAb).

In certain embodiments, the TCR fusion proteins may comprise one or more additional biologically active moieties that are cytokines or biologically active fragments thereof.

### Formats

The TCR fusion proteins of the present disclosure may have various formats. Within a TCR fusion protein, a TCR construct may be fused or covalently attached to a second element of the fusion protein via the TCR alpha chain polypeptide or the TCR beta chain polypeptide, or both. For example, a TCR construct may be fused or covalently attached to a scaffold, to an additional biologically active moiety or to another TCR construct via the TCR alpha chain polypeptide or the TCR beta chain polypeptide, or both. The TCR construct may be fused or covalently attached to a second element of the fusion protein through the N-terminus or the C-terminus of the relevant TCR polypeptide(s) and may be directly or indirectly (for example, by way of a linker) attached to the second element.

In certain embodiments, the TCR fusion protein comprises a TCR construct that is fused or covalently attached to a scaffold or additional biologically active moiety via the TCR beta chain polypeptide. In certain embodiments, the TCR fusion protein comprises a TCR construct that is fused or covalently attached to a scaffold or additional biologically active moiety via the C-terminus of one of the polypeptides of the TCR construct. In certain embodiments, the TCR fusion protein comprises a TCR construct that is fused or covalently attached to a scaffold or additional biologically active moiety via the C-terminus of the TCR beta chain polypeptide of the TCR construct.

In those embodiments in which the TCR fusion protein comprises a scaffold and multiple TCR constructs, the TCR constructs may be fused or covalently attached to the scaffold in tandem or they may be fused or covalently attached to different parts of the scaffold. In those embodiments in which the TCR fusion protein comprises a scaffold, a TCR construct and an additional biologically active moiety, the TCR construct and additional biologically active moiety may be fused or covalently attached to the scaffold in tandem or they may be fused or covalently attached to different parts of the scaffold. In those embodiments in which the TCR fusion protein comprises more than two TCR constructs, some of the TCR constructs may be fused or covalently attached to the scaffold in tandem and others may be fused or covalently attached to different parts of the scaffold, or all may be fused or covalently attached to different parts of the scaffold.

In certain embodiments, the TCR fusion proteins of the present disclosure comprise a TCR construct and an additional biologically active moiety. In some embodiments, the TCR fusion proteins comprise a TCR construct fused directly or indirectly to an antigen-binding domain or a cytokine.

In certain embodiments, the TCR fusion proteins of the present disclosure comprise one or more TCR constructs and a scaffold. In some embodiments, the TCR fusion proteins comprise one or more TCR constructs and an Ig Fc scaffold. In some embodiments, the TCR fusion proteins comprise one or more TCR constructs, an Ig Fc scaffold and one or more additional biologically active moieties. In some embodiments, the TCR fusion proteins comprise one or more TCR constructs, an Ig Fc scaffold and one or more antigen-binding domains. In some embodiments, the TCR fusion proteins comprise between 1 and 12 TCR constructs, an Ig Fc scaffold and between 1 and 6 antigen-binding domains. In some embodiments, the TCR fusion proteins comprise between 1 and 8 TCR constructs, an Ig Fc scaffold and between 1 and 6 antigen-binding domains. In some embodiments, the TCR fusion proteins comprise between 1 and 6 TCR constructs, an Ig Fc scaffold and between 1 and 4 antigen-binding domains.

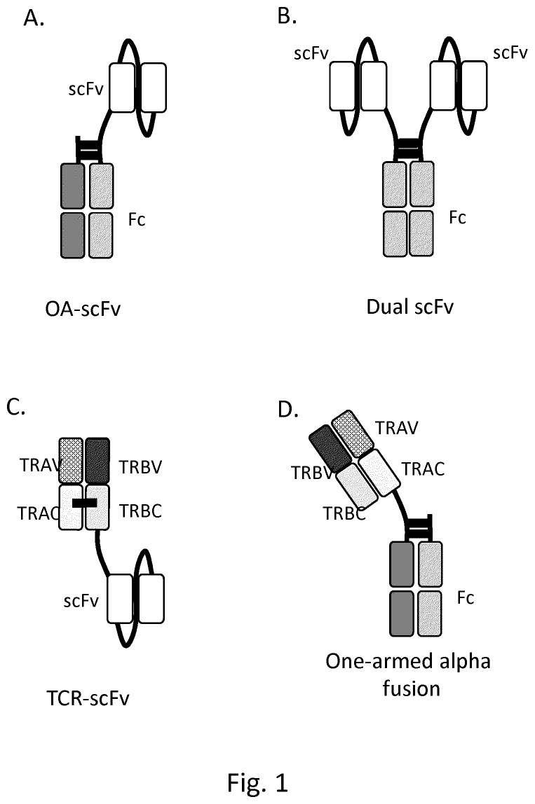

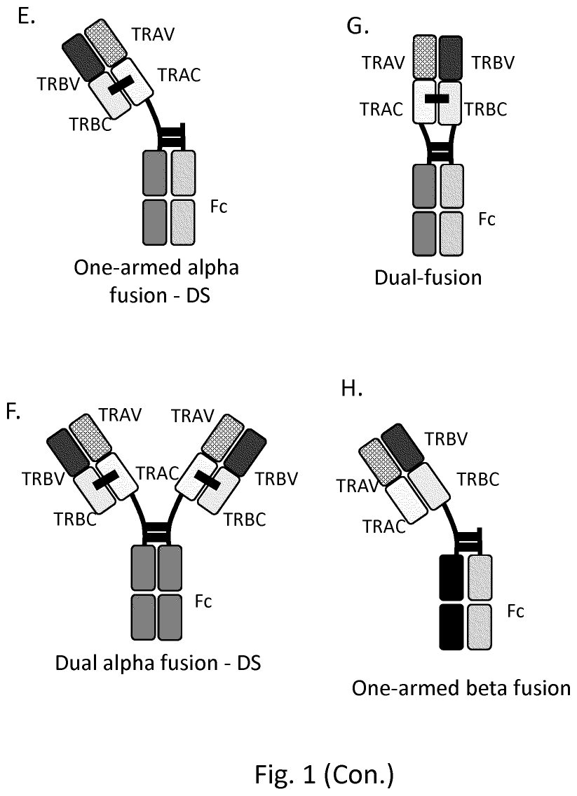

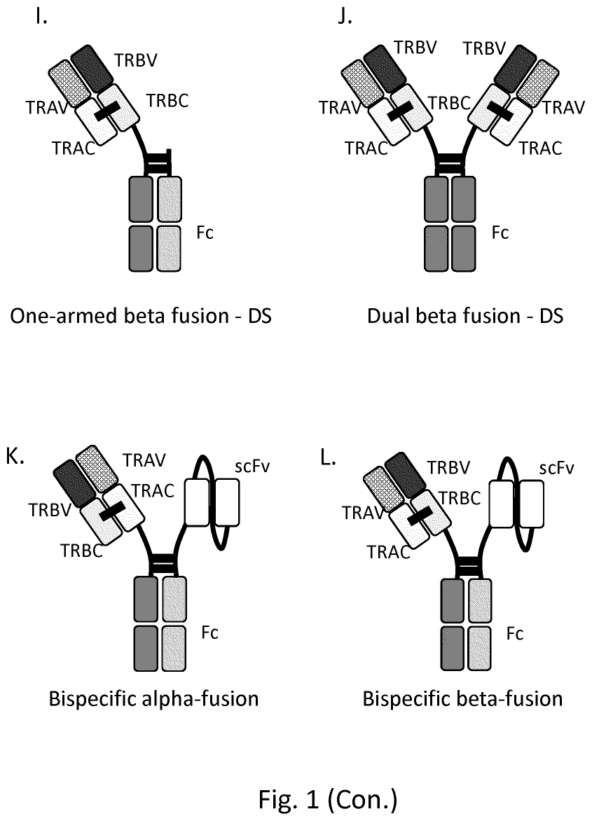

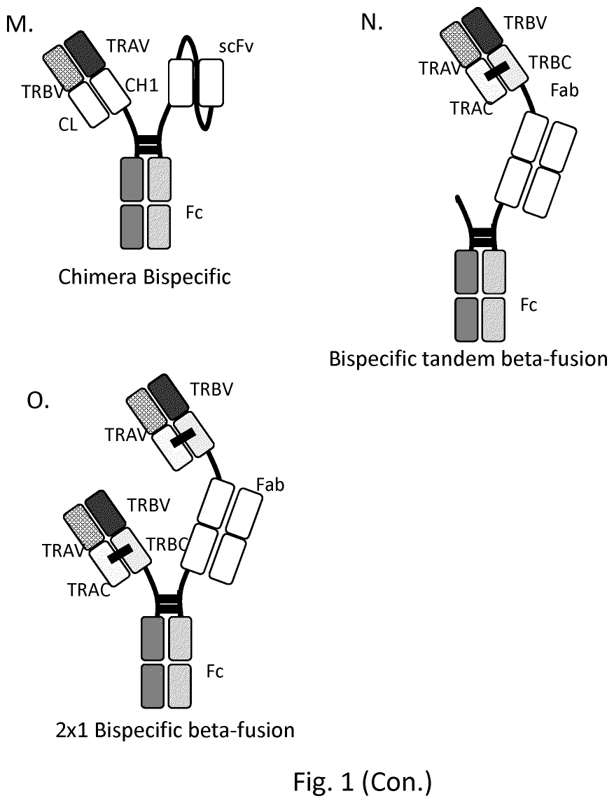

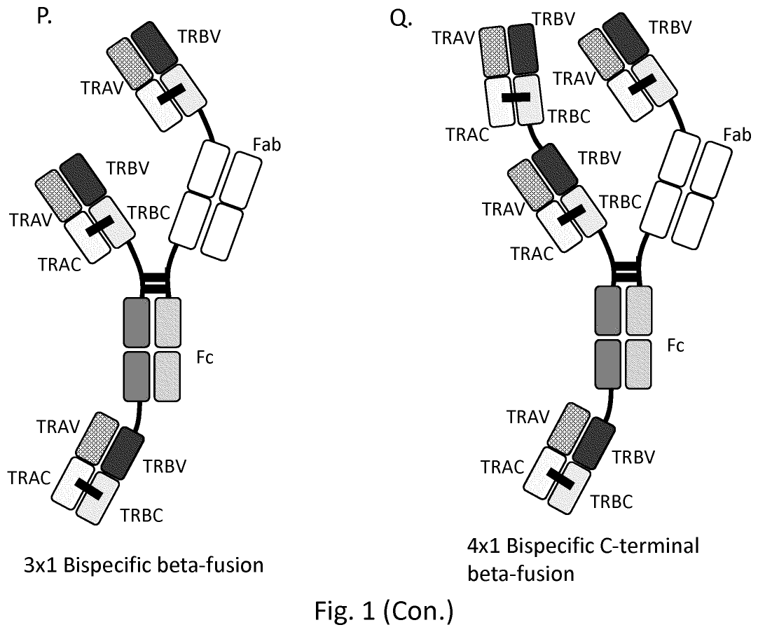

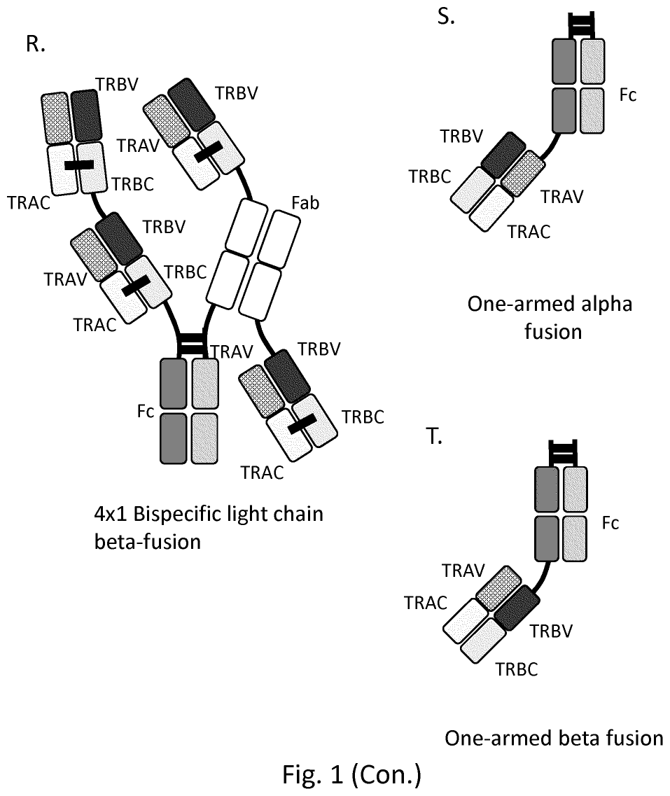

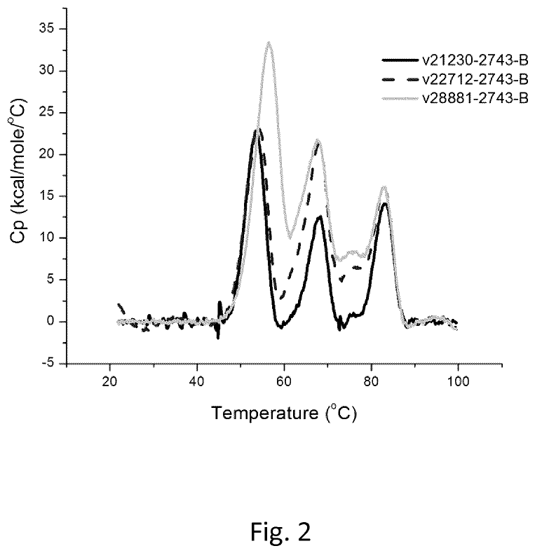

FIG. 1 shows various non-limiting examples of formats for TCR fusion proteins comprising an IgG Fc as a scaffold and one or more TCR constructs and non-limiting examples of formats for TCR fusion proteins comprising an IgG Fc as a scaffold, one or more TCR constructs and one or more additional biologically active moieties (illustrated by antigen-binding domains). FIG. 1(C) shows a TCR fusion protein comprising a TCR fused to an antigen-binding domain (scFv) (“TCR-scFv”). FIG. 1(D) and 1(E) show a one-armed TCR fusion protein with the TCR alpha polypeptide fused an Fc scaffold (“One-armed alpha fusion” (D) and “One-armed alpha fusion-DS” (E), which includes an interchain disulfide bond). FIG. 1(F) shows a TCR fusion protein comprising two TCR constructs each independently fused to a polypeptide of an Fc scaffold via the TCR alpha polypeptide and each including an interchain disulfide bond between the TCR alpha polypeptide and the TCR beta polypeptide (“Dual alpha fusion-DS”). FIG. 1(G) shows a TCR fusion protein with each of the TCR alpha polypeptide and TCR beta polypeptide independently fused to an Fc scaffold (“Dual-fusion”). FIGS. 1(H) and (I) show a one-armed TCR fusion protein with the TCR beta polypeptide fused to an Fc scaffold (“One-armed beta fusion” (H) and “One-armed beta fusion-DS” (I), which includes an interchain disulfide bond between the TCR alpha and beta polypeptides). FIG. 1(J) shows a TCR fusion protein comprising two TCR constructs each independently fused to an Fc scaffold via the TCR beta polypeptide and each including an interchain disulfide bond between the TCR alpha polypeptide and the TCR beta polypeptide (“Dual beta fusion-DS”). FIG. 1(K) shows a TCR fusion protein comprising a TCR construct fused to one polypeptide of an Fc scaffold via the TCR alpha chain and an scFv fused to the other Fc polypeptide, the TCR includes an interchain disulfide bond between the TCR alpha and beta polypeptides (“Bispecific alpha fusion”). FIG. 1(L) shows a TCR fusion protein comprising a TCR construct fused to one polypeptide of an Fc scaffold via the TCR beta polypeptide and an scFv fused to the other Fc polypeptide, the TCR includes an interchain disulfide bond between the TCR alpha and beta polypeptides (“Bispecific beta fusion”). FIG. 1(M) shows a TCR fusion protein in which one arm comprises a TRAV domain fused to a CH1 domain and a TRBC domain fused to a CL domain and a second arm comprises an scFv, each arm independently fused to a polypeptide of an Fc scaffold (“Chimera Bispecific”). FIG. 1(N) shows a one-armed TCR fusion protein comprising a Fab fused to one polypeptide of an Fc scaffold and a TCR construct fused to the Fab via the TCR beta polypeptide, the TCR construct including an interchain disulfide bond between the TCR alpha and beta polypeptides (“Bispecific tandem beta-fusion”). FIG. 1(O) shows a TCR fusion protein comprising a Fab fused to one polypeptide of an Fc scaffold, a first TCR construct fused to the Fab via the TCR beta polypeptide, and a second TCR construct fused to the other Fc polypeptide via the TCR beta polypeptide, each TCR including an interchain disulfide bond between the TCR alpha and beta polypeptides (“2×1 Bispecific beta-fusion”). FIG. 1(P) shows a TCR fusion protein comprising a Fab fused to one polypeptide of an Fc scaffold, a first TCR construct fused to the Fab via the TCR beta polypeptide, a second TCR construct fused to the second Fc polypeptide via the TCR beta polypeptide, and a third TCR construct fused to the C-terminus of the second Fc polypeptide via the TCR beta polypeptide, each TCR including an interchain disulfide bond between the TCR alpha and beta polypeptides (“3×1 Bispecific beta-fusion”). FIG. 1(Q) shows a TCR fusion protein comprising a Fab fused to one polypeptide of an Fc scaffold, a first TCR construct fused to the Fab via the TCR beta polypeptide, a second TCR construct fused to the second Fc polypeptide via the TCR beta polypeptide, a third TCR construct fused to the second TCR via the TCR beta polypeptide, and a fourth TCR construct fused to the C-terminus of the second Fc polypeptide via the TCR beta polypeptide, each TCR including an interchain disulfide bond between the TCR alpha and beta polypeptides (“4×1 Bispecific C-terminal beta-fusion”). FIG. 1(R) shows a TCR fusion protein comprising a Fab fused to one polypeptide of an Fc scaffold, a first TCR construct fused to the Fab via the TCR beta polypeptide, a second TCR construct fused to the second Fc polypeptide via the TCR beta polypeptide, a third TCR construct fused to the second TCR via the TCR beta polypeptide, and a fourth TCR construct fused to the C-terminus of the light chain of the Fab, each TCR including an interchain disulfide bond between the TCR alpha and beta polypeptides (“4×1 Bispecific light chain beta-fusion”). FIG. 1(S) shows a TCR fusion protein comprising a TCR fused to the C-terminus of one polypeptide of an Fc scaffold via the TCR alpha polypeptide (“One-armed alpha fusion”). FIG. 1(T) shows a TCR fusion protein comprising a TCR fused to the C-terminus of one polypeptide of an Fc scaffold via the TCR beta polypeptide (“One-armed beta fusion”).

The embodiments of TCR fusion proteins shown in FIG. 1 are illustrative only and not limiting. Other conformations are contemplated and encompassed by the present disclosure including TCR fusion proteins as illustrated in FIG. 1 employing other antigen-binding domains or other biologically active moieties to replace those illustrated, as well as TCR constructs including stabilizing mutations as described herein other than or in addition to the illustrated interchain disulfide bond.

### Preparation of TCR Constructs and TCR Fusion Proteins

The TCR constructs and TCR fusion proteins described herein may be produced using standard recombinant methods known in the art. Typically, for recombinant production of a TCR construct or TCR fusion protein, a polynucleotide or set of polynucleotides encoding the TCR construct or TCR fusion protein is generated and inserted into one or more vectors for further cloning and/or expression in a host cell. Polynucleotide(s) encoding the TCR construct or TCR fusion protein may be produced by standard methods known in the art (see, for example, Ausubel et al., Current Protocols in Molecular Biology, John Wiley & Sons, New York, 1994 & updates, and “Antibodies: A Laboratory Manual,” 2^{nd} Edition, Ed. Greenfield, Cold Spring Harbor Laboratory Press, New York, 2014). The number of polynucleotides required for expression of the TCR construct or TCR fusion protein will be dependent on the format of the construct or protein, including whether or not the construct comprises a scaffold. When multiple polynucleotides are required, they may be incorporated into one vector (e.g. a multicistronic vector) or into more than one vector.

Generally, for expression, the polynucleotide or set of polynucleotides encoding the TCR construct or TCR fusion protein is incorporated into an expression vector together with one or more regulatory elements, such as transcriptional elements, which are required for efficient transcription of the polynucleotide. Examples of such regulatory elements include, but are not limited to, promoters, enhancers, terminators, and polyadenylation signals. One skilled in the art will appreciate that the choice of regulatory elements is dependent on the host cell selected for expression of the TCR construct or TCR fusion protein and that such regulatory elements may be derived from a variety of sources, including bacterial, fungal, viral, mammalian or insect genes. The expression vector may optionally further contain heterologous nucleic acid sequences that facilitate expression or purification of the expressed protein. Examples include, but are not limited to, signal peptides and affinity tags such as metal-affinity tags, histidine tags, avidin/streptavidin encoding sequences, glutathione-S-transferase (GST) encoding sequences and biotin encoding sequences. The expression vector may be an extrachromosomal vector or an integrating vector.

Suitable host cells for cloning or expression of the TCR construct or TCR fusion protein include various prokaryotic or eukaryotic cells as known in the art. Prokaryotic host cells include, for example, E. coli, A. salmonicida or B. subtilis cells. Eukaryotic host cells include, for example, mammalian cells, plant cells, insect cells and yeast cells (such as Saccharomyces or Pichia cells). Eukaryotic microbes such as filamentous fungi or yeast may be suitable expression host cells in certain embodiments. Fungi and yeast strains whose glycosylation pathways have been “humanized” resulting in the production of an antibody construct with a partially or fully human glycosylation pattern have been developed (see, for example, Gerngross, 2004, Nat. Biotech. 22:1409-1414, and Li et al., 2006, Nat. Biotech. 24:210-215) and may be useful in certain embodiments.

In certain embodiments, the TCR construct or TCR fusion protein is expressed in eukaryotic host cells. In some embodiments, the TCR construct or TCR fusion protein is expressed in a mammalian cell line. Mammalian cell lines adapted to grow in suspension are particularly useful in this regard. Examples include, but are not limited to, monkey kidney CV1 line transformed by SV40 (COS-7), human embryonic kidney (HEK) line 293 (“293 cells”) (see, for example, Graham et al., 1977, J. Gen Virol., 36:59), baby hamster kidney cells (BHK), mouse sertoli TM4 cells (see, for example, Mather, 1980, Biol Reprod, 23:243-251), monkey kidney cells (CV1), African green monkey kidney cells (VERO-76), human cervical carcinoma (HeLa) cells, canine kidney cells (MDCK), buffalo rat liver cells (BRL 3A), human lung cells (W138), human liver cells (Hep G2), mouse mammary tumour cells (MMT 060562), TRI cells (see, for example, Mather et al., 1982, Annals N.Y. Acad Sci, 383:44-68), MRC 5 cells, FS4 cells, Chinese hamster ovary (CHO) cells (including DHFR-CHO cells, see Urlaub et al., 1980, Proc Natl Acad Sci USA, 77:4216), and myeloma cell lines (such as Y0, NS0 and Sp2/0). Exemplary mammalian host cell lines are reviewed in Yazaki & Wu, Methods in Molecular Biology, Vol. 248, pp. 255-268 (B.K.C. Lo, ed., Humana Press, Totowa, N.J., 2003).

In certain embodiments, the TCR construct or TCR fusion protein is expressed in a transient or stable mammalian cell line. In some embodiments, the TCR construct or TCR fusion protein is expressed in HEK293, CHO, HeLa, NS0 or COS cells. In some embodiments, the TCR construct or TCR fusion protein is expressed in HEK293 cells.

The host cells comprising the expression vector(s) encoding the TCR construct or TCR fusion protein may be cultured using routine methods to produce the TCR construct or TCR fusion protein. In certain embodiments, culturing the host cells comprising the expression vector(s) encoding the TCR construct or TCR fusion protein at a lowered temperature may improve expression and/or decrease the amount of HM/1W species (aggregation) of the TCR construct or TCR fusion protein. In certain embodiments, the host cells comprising the expression vector(s) encoding the TCR construct or TCR fusion protein may be cultured at a temperature below 37° C.

In some embodiments, the host cells comprising the expression vector(s) encoding the TCR construct or TCR fusion protein may be cultured at a temperature between about 30° C. and about 36° C., for example between about 30° C. and about 35° C., or between about 30° C. and about 34° C.

In some embodiments, the host cells comprising the expression vector(s) encoding the TCR construct or TCR fusion protein may be cultured at a temperature of about 32° C.

Typically, the TCR constructs and TCR fusion proteins are purified after expression. Proteins may be isolated or purified in a variety of ways known to those skilled in the art (see, for example, Protein Purification: Principles and Practice, 3^{rd} Ed., Scopes, Springer-Verlag, NY, 1994). Standard purification methods include chromatographic techniques, including ion exchange, hydrophobic interaction, affinity, sizing or gel filtration, and reverse-phase, carried out at atmospheric pressure or at high pressure using systems such as FPLC and HPLC. Additional purification methods include electrophoretic, immunological, precipitation, dialysis and chromatofocusing techniques. Ultrafiltration and diafiltration techniques, in conjunction with protein concentration, may also be useful.

Purification may also be facilitated by a particular fusion partner. For example, TCR constructs and TCR fusion proteins may be purified using glutathione resin if a GST fusion is employed, by Ni⁺² affinity chromatography if a His-tag is employed, or by immobilized anti-flag antibody if a flag-tag is used.

In certain embodiments in which the TCR fusion protein comprises an Ig Fc scaffold and/or an antibody Fab region, purification may comprise the use of one of a variety of natural proteins that bind the Fc or Fab. For example, the bacterial proteins A and G bind to the Fc region. Likewise, the bacterial protein L binds to the Fab region of some antibodies.

The degree of purification necessary will vary depending on the intended use of the TCR construct or TCR fusion protein. In some instances, no purification may be necessary. In certain embodiments, the TCR constructs and TCR fusion proteins are substantially pure. The term “substantially pure” (or “substantially purified”) when used in reference to a TCR construct or TCR fusion protein described herein, means that the TCR construct or TCR fusion protein is substantially or essentially free of components that normally accompany or interact with the protein in the host cell in which it is expressed. In certain embodiments, a substantially pure preparation of a TCR construct or TCR fusion protein is a protein preparation having less than about 10% of contaminating protein. By contaminating protein in this context, it is meant any protein that is not the TCR construct or TCR fusion protein, but does not include aggregated forms of the TCR construct or TCR fusion protein (i.e. HIW species). In some embodiments, a substantially pure preparation of a TCR construct or TCR fusion protein is a protein preparation having less than about 8% of contaminating protein, for example, less than about 7%, less than about 6%, or less than about 5%, of contaminating protein.

In certain embodiments, the TCR construct or TCR fusion protein preparation comprises minimal amount of HMIW species (aggregates). In some embodiments, a TCR construct or TCR fusion protein preparation comprises about 40% or less of HMW species. In some embodiments, a TCR construct or TCR fusion protein preparation comprises about 35% or less of HMW species, for example, about 30% or less, about 25% or less, about 20% or less, or about 15% or less, of HMW species. In certain embodiments, the amount of HMW species is determined by size-exclusion chromatography (SEC), for example, by UPLC-SEC.

Certain embodiments of the present disclosure relate to a method of preparing a TCR construct or TCR fusion protein as described herein comprising culturing a host cell into which one or more polynucleotides, or one or more expression vectors, encoding the TCR construct or TCR fusion protein have been introduced, under conditions suitable for expression of the TCR construct or TCR fusion protein, and optionally recovering the TCR construct or TCR fusion protein from the host cell (or from host cell culture medium). In certain embodiments, the method comprises culturing the host cell comprising the polynucleotide(s) or expression vector(s) encoding the TCR construct or TCR fusion protein at a temperature below 37° C. In some embodiments, the method comprises culturing the host cell comprising the polynucleotide(s) or expression vector(s) encoding the TCR construct or TCR fusion protein at a temperature between about 30° C. and about 36° C., for example between about 30° C. and about 35° C., or between about 30° C. and about 34° C. In some embodiments, the method comprises culturing the host cell comprising the polynucleotide(s) or expression vector(s) encoding the TCR construct or TCR fusion protein at a temperature of about 32° C. In certain embodiments, the host cell is a human embryonic kidney (HEK) cell, such as a HEK273 cell.

### Polynucleotides, Vectors and Host Cells

Certain embodiments of the present disclosure relate to an isolated polynucleotide or set of polynucleotides encoding a TCR construct or TCR fusion protein described herein. A polynucleotide in this context may encode all or a part of a TCR construct or TCR fusion protein.

The terms “nucleic acid,” “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, isolated DNA, isolated RNA, nucleic acid probes, and primers.

A polynucleotide that “encodes” a given polypeptide is a polynucleotide that is transcribed (in the case of DNA) or translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

Certain embodiments of the present disclosure relate to vectors (such as expression vectors) comprising one or more polynucleotides encoding a TCR construct or TCR fusion protein as described herein. The polynucleotide(s) may be comprised by a single vector or by more than one vector. In some embodiments, the polynucleotides are comprised by a multicistronic vector.

Certain embodiments of the present disclosure relate to host cells comprising polynucleotide(s) encoding a TCR construct or TCR fusion protein or one or more vectors comprising the polynucleotide(s). In some embodiments, the host cell is eukaryotic, for example, a mammalian cell. In some embodiments, the host cell is a human cell. In some embodiments, the host cell is a human embryonic kidney (HEK) cell.

### Pharmaceutical Compositions

For therapeutic use, the TCR constructs and TCR fusion proteins may be provided in the form of compositions comprising the TCR construct or TCR fusion protein and a pharmaceutically acceptable carrier or diluent. The compositions may be prepared by known procedures using well-known and readily available ingredients.

Pharmaceutical compositions may be formulated for administration to a subject by, for example, oral (including, for example, buccal or sublingual), topical, parenteral, rectal or vaginal routes, or by inhalation or spray. The term “parenteral” as used herein includes subcutaneous injection, and intradermal, intra-articular, intravenous, intramuscular, intravascular, intrasternal, intrathecal injection or infusion. The pharmaceutical composition will typically be formulated in a format suitable for administration to the subject, for example, as a syrup, elixir, tablet, troche, lozenge, hard or soft capsule, pill, suppository, oily or aqueous suspension, dispersible powder or granule, emulsion, injectable or solution. Pharmaceutical compositions may be provided as unit dosage formulations.

In certain embodiments, the pharmaceutical compositions comprising the TCR constructs or TCR fusion proteins are formulated for parenteral administration. In some embodiments, the pharmaceutical compositions comprising the TCR constructs or TCR fusion proteins are formulated for parenteral administration in a unit dosage injectable form, for example as lyophilized formulations or aqueous solutions.

Pharmaceutically acceptable carriers and diluents are generally nontoxic to recipients at the dosages and concentrations employed. Examples of components that may be included in such carriers and diluents include, but are not limited to, buffers such as phosphate, citrate and other organic acids; antioxidants such as ascorbic acid and methionine; preservatives such as octadecyldimethylbenzyl ammonium chloride, hexamethonium chloride, benzalkonium chloride, benzethonium chloride, phenol, butyl alcohol, benzyl alcohol, alkyl parabens (such as methyl or propyl paraben), catechol, resorcinol, cyclohexanol, 3-pentanol and m-cresol; low molecular weight (less than about 10 residues) polypeptides; proteins such as serum albumin or gelatin; hydrophilic polymers such as polyvinylpyrrolidone; amino acids such as glycine, glutamine, asparagine, histidine, arginine or lysine; monosaccharides, disaccharides, and other carbohydrates such as glucose, mannose or dextrins; chelating agents such as EDTA; sugars such as sucrose, mannitol, trehalose or sorbitol; salt-forming counter-ions such as sodium; metal complexes such as Zn-protein complexes, and non-ionic surfactants such as polyethylene glycol (PEG).

In certain embodiments, the compositions comprising the TCR constructs or TCR fusion proteins may be in the form of a sterile injectable aqueous or oleaginous solution or suspension. Such suspensions may be formulated using suitable dispersing or wetting agents and/or suspending agents that are known in the art. The sterile injectable solution or suspension may comprise the TCR construct or TCR fusion protein in a non-toxic parentally acceptable carrier or diluent. Acceptable carriers and diluents that may be employed include, for example, 1,3-butanediol, water, Ringer's solution or isotonic sodium chloride solution. In addition, sterile, fixed oils may be employed. For this purpose, various bland fixed oils may be employed, including synthetic mono-or diglycerides. In addition, fatty acids such as oleic acid find use in the preparation of injectables. Adjuvants such as local anaesthetics, preservatives and/or buffering agents may also be included in the injectable solution or suspension.

In certain embodiments, the composition comprising the TCR construct or TCR fusion protein may be formulated for intravenous administration to a subject. Typically, compositions for intravenous administration are solutions in sterile isotonic aqueous buffer. Where necessary, the composition may also include a solubilizing agent and/or a local anaesthetic such as lignocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it may be dispensed with an infusion bottle containing sterile pharmaceutical grade water or saline. Where the composition is administered by injection, an ampoule of sterile water for injection or saline may be provided so that the ingredients may be mixed prior to administration.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### Methods of Use

Certain embodiments of the present disclosure relate to therapeutic uses of the TCR constructs and TCR fusion proteins described herein. For example, TCR constructs and TCR fusion proteins as described herein may be used to target various types of disease cells or infected cells, or a specific tissue type or organ. Accordingly, certain embodiments of the present disclosure relate to methods for the treatment of a disease or condition comprising administration of a TCR construct or TCR fusion protein to a subject in need thereof. In some embodiments, the subject is a mammal. In some embodiments, the subject is a human.

Various diseases, disorders and conditions may be treated depending on the specific disease cell, infected cell, tissue or organ being targeted by the TCR construct or TCR fusion protein. Examples include, but are not limited to, cancer, bacterial infection, viral infection, infectious diseases, and immune disorders including immunodeficiency disorders and diseases, and auto-immune diseases and conditions. In some embodiments, the TCR constructs and TCR fusion proteins may be used in methods for the treatment of cancer. In some embodiments, the TCR constructs and TCR fusion proteins may be used in methods for the treatment of a bacterial or viral infection, or an infectious disease. In some embodiments, the TCR constructs and TCR fusion proteins may be used in methods for the treatment of an immune disorder. In some embodiments, the TCR constructs and TCR fusion proteins may be used in methods for the treatment of an immunodeficiency disorder or disease. In certain embodiments, the TCR constructs and TCR fusion proteins may be used in methods for the treatment of an auto-immune disease or condition.

The methods of treatment described herein comprise administering the TCR construct or TCR fusion protein to a subject in need thereof. The TCR construct or TCR fusion protein will be administered to a subject by an appropriate route of administration. The route and/or mode of administration will vary depending upon the disease or condition to be treated and the desired results, and can be readily determined by one skilled in the medical arts.

Alternatively, in some embodiments, host cells comprising expression vector(s) encoding the TCR construct or TCR fusion protein may be used therapeutically or prophylactically to deliver the TCR construct or TCR fusion protein to a subject, or polynucleotides or expression vectors encoding the TCR construct or TCR fusion protein may be administered to a cell from a subject ex vivo and the cell then returned to the body of the subject.

Treatment is achieved by administration of a “therapeutically effective amount” of the TCR construct or TCR fusion protein. A “therapeutically effective amount” refers to an amount that is effective, at dosages and for periods of time necessary, to achieve a desired therapeutic result. A therapeutically effective amount can vary according to factors such as the disease state, age, sex, and weight of the subject. A therapeutically effective amount may also be one in which any toxic or detrimental effects of the TCR construct or TCR fusion protein are outweighed by the therapeutically beneficial effects.

A suitable dosage of the TCR construct or TCR fusion protein can be determined by a skilled medical practitioner. The selected dosage level will depend upon a variety of pharmacokinetic factors including the activity of the particular TCR construct or TCR fusion protein employed, the route of administration, the time of administration, the rate of excretion of the construct or protein, the duration of the treatment, other drugs, compounds and/or materials used in combination with the TCR construct or TCR fusion protein, the age, sex, weight, condition, general health and prior medical history of the subject being treated, and like factors well known in the medical arts.

Certain embodiments of the present disclosure relate to diagnostic uses of the TCR constructs and TCR fusion proteins. For example, the TCR construct or TCR fusion protein may be used in vitro or in vivo to detect antigen-presenting cells carrying the peptide for which the TCR construct or TCR fusion protein specifically binds. For diagnostic uses, the TCR construct or TCR fusion protein is typically labelled with an appropriate detectable label.

The following Examples are provided for illustrative purposes and are not intended to limit the scope of the present disclosure in any way.

### EXAMPLES

### Example 1: Design and Cloning of TCR Fusion Proteins

TCR fusion proteins and controls in various formats are described in the following Examples. The design and construction of the TCR fusion proteins and controls is outlined below.

All TCR fusion proteins contained the extracellular Vα and Vβ TCR domains together with either the extracellular Cα and Cβ TCR domains or IgG1 CL and CH domains, and an IgG1 Fc. Controls also contained the extracellular TCR domains and an IgG1 Fc, unless otherwise indicated. Amino acid residues in the Fc region are numbered according to the EU index. Amino acid residues in the extracellular TCR domains are numbered according to the IMGT numbering system (Lefranc, et al., 2005, Developmental and Comparative Immunology, 29:185-203; see FIG. 9).

To optimize preparation of the TCR fusion proteins, the IgG1 Fc comprised CH3 domain amino acid substitutions that promote formation of a heterodimeric Fc. Specifically, the IgG1 Fc contained by the TCR fusion proteins was a human IgG1 heterodimeric Fc comprising the following CH3 domain amino acid substitutions (referred to throughout the Examples as “Het Fc” modifications):

Chain A (“Het FcA”): T350V/L351Y/F405A/Y407V

Chain B (“Het FcB”): T350V/T366L/K392L/T394W.

For some TCR fusion proteins, both chains of the IgG1 Fc also comprised the following CH2 amino acid substitutions which abrogate FcTR binding (referred to throughout the Examples as “FcKO”): L234A/L235A/D265S.

Monovalent, bivalent, trivalent and tetravalent TCR fusion proteins were prepared. A schematic depiction of each TCR fusion protein format is shown in FIG. 1. In order to produce each of the final TCR fusion proteins in the formats shown in FIG. 1, the following heavy chain constructs were prepared (from N-terminus to C-terminus):
a) Vα-Cα-hinge-CH2-CH3
b) Vβ-Cβ-hinge-CH2-CH3
c) Vα-Cα-hinge-CH2-CH3
d) Vβ-Cβ-hinge-CH2-CH3-Vβ-Cβ-hinge
e) Vβ-Cβ-hinge-Vβ-Cβ-hinge-CH2-CH3-Vβ-Cβ-hinge
f) Vβ-Cβ-hinge-VH-CH1-CH2-CH3
g) Vβ-Cβ-hinge-VH-CH1-CH2-CH3-Vβ-Cβ-hinge
h) VL-VH-hinge-CH2-CH3
i) Hinge-CH2-CH3.

All heavy chain constructs included the HetFc modifications. In addition to two of the heavy chains described above, most TCR fusion proteins also comprised a complementary alpha or beta TCR chain containing the corresponding variable and constant domains. Those TCR fusion proteins comprising an antibody Fab further comprised a single antibody light chain.

The “hinge” sequence corresponds to the upper region of the human IgG1 hinge sequence (EPKSCDKTHT [SEQ ID NO:16]) except when “hinge” is followed by “CH2,” in which case the lower region of the human IgG1 hinge was also included (i.e. the sequence was EPKSCDKTHTCPPCP [SEQ ID NO:21]). The TCR α chain consists of the extracellular Vα and Cα domains. The sequence terminates at TRAC/127. The TCR β chain consists of the extracellular Vβ and Cβ domains. The sequence terminates at TRCB/126. The wild-type TCR β chain constant region includes a cysteine residue (residue 85.1 in exon 1 of TRBC1*01 and TRBC2*01) which is not involved in either inter-chain or intra-chain disulfide bond formation. This position is commonly mutated to an Ala to eliminate potential mispairing. All TCR fusion proteins described in the following Examples incorporate this TRBC/85.1.CYS->ALA mutation.

As noted in the following Examples, some of the TCR fusion proteins include the known TRAC/84.THR-TRBC/79.SER disulfide bond, which has been shown to improve the expression and stability of TCR proteins (Boulter, et al., 2003, PEDS, 16:707-711). The TRAC/84.THR-TRBC/79.SER disulfide bond is referred to throughout the Examples as the “IC Disulfide.”

As also noted in the following Examples, some of the TCR fusion proteins include the mutations TRAC/1.5.GLN->LYS and TRBC/97.GLN->ASP. These mutations do not alter either the activity or the stability of the TCR.

Table 1.1 provides a summary of the TCR fusion proteins that were prepared. The number of TCR “arms” (peptide-TCC targeting domains) and the number of anti-CD3 “arms” (CD3 targeting domains) comprised by each TCR fusion protein are indicated in the “Format” column. For example, “1×0” indicates that the TCR fusion protein comprises one TCR arm and no anti-CD3 arms, “1×1” indicates that the TCR fusion protein comprises one TCR arm and one anti-CD3 arm, etc. Anti-CD3 arms were in either scFv or Fab format as noted.

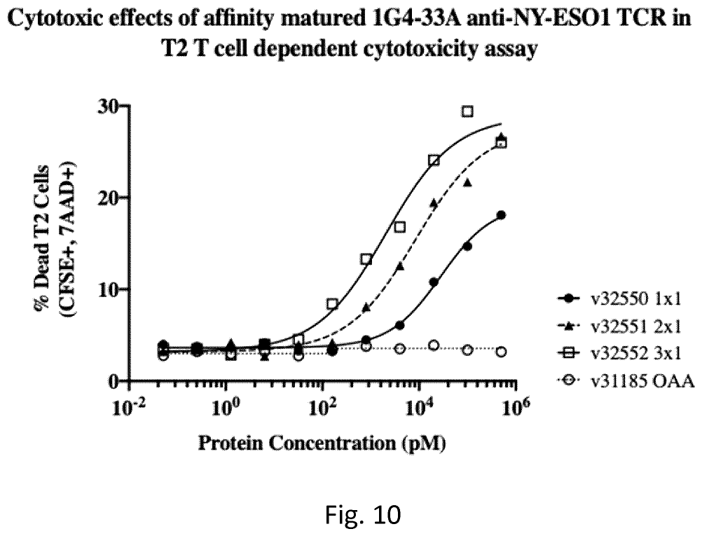

TABLE 1.1
Exemplary TCR Fusion Protein Formats

| | Format | TCR | | |
|---|---|---|---|---|
| | (TCR x | Chain | Fc | Description |
| Paratopes | anti-CD3) | Fusion | Description | (Cross-reference to Fig. 1) |
| Monovalent TCR | 1 × 0 | β | Het Fc, | TCR one-armed construct |
| | | | FcKO | (“One-armed beta fusion”; Fig. |
| | | | | 1H and 1I) |
| Monovalent TCR | 1 × 0 | α | Het Fc, | TCR one-armed construct |
| | | | FcKO | (“One-armed alpha fusion”; Fig. |
| | | | | 1D and 1E) |
| Monovalent TCR | 1 × 0 | α | Het Fc, | TCR one-armed construct. C- |
| | | | FcKO | terminal fusion |
| | | | | (“C-terminal one-armed alpha |
| | | | | fusion”; Fig. 1S) |
| Monovalent TCR | 1 × 0 | β | Het Fc, | TCR one-armed construct. C- |
| | | | FcKO | terminal fusion |
| | | | | (“C-terminal one-armed beta |
| | | | | fusion”; Fig. 1T) |
| Monovalent TCR | 1 × 0 | α + β | Het Fc, | TCR dual fusion |
| | | | FcKO | (“Dual fusion”; Fig 1G) |
| Monovalent TCR, | 1 × 1 | β | Het Fc, | TCR x anti-CD3 bispecific fusion |
| monovalent anti- | | | FcKO | protein |
| CD3 scFv | | | | (“Bispecific beta-fusion”; Fig. |
| | | | | 1L) |
| Monovalent TCR, | 1 × 1 | β | No Fc | TCR x anti-CD3 bispecific fusion |
| monovalent anti- | | | | protein control construct. TCR |
| CD3 scFv | | | | fused to scFv with no Fc |
| | | | | (″TCR-scFv”; Fig. 1C) |
| Monovalent TCR, | 1 × 1 | β | Het Fc, | TCR x anti-CD3 bispecific |
| monovalent anti- | | | FcKO | tandem fusion protein |
| CD3 Fab | | | | (“Bispecific tandem beta-fusion”; |
| | | | | Fig. 1N) |
| Bivalent TCR, | 2 × 1 | β | Het Fc, | TCR x anti-CD3 bispecific fusion |
| monovalent anti- | | | FcKO | protein |
| CD3 Fab | | | | (“2 × 1 Bispecific beta-fusion”; |
| | | | | Fig. 10) |
| Trivalent TCR, | 3 × 1 | β | Het Fc, | TCR x anti-CD3 bispecific fusion |
| monovalent anti- | | | FcKO | protein |
| CD3 Fab | | | | (“3 × 1 Bispecific beta-fusion”; |
| | | | | Fig. 1P) |
| Tetravalent TCR, | 4 × 1 | β | Het Fc, | TCR x anti-CD3 bispecific fusion |
| monovalent anti- | | | FcKO | protein |
| CD3 Fab | | | | (“4 × 1 Bispecific C-terminal beta- |
| | | | | fusion”; Fig. 1Q) |
| Tetravalent TCR, | 4 × 1 | β | Het Fc, | TCR x anti-CD3 bispecific fusion |
| monovalent anti- | | | FcKO | protein, and LC fusion |
| CD3 Fab | | | | (4 × 1 Bispecific light chain beta- |
| | | | | fusion”; Fig. 1R) |
| Monovalent anti- | 0 × 1 | β | Het Fc, | Control CD3-binding antibody |
| CD3 scFv | | | FcKO | (“OA-scFv”; Fig. 1A) |

Vectors for the expression of the TCR fusion proteins were constructed as follows. All constructs used the pTT5 vector (Durocher, et al., 2002, Nucl. Acids Res., 30(2):e9) and the following signal sequence: MIRPTWAWWLFLVLLLALWAPARG [SEQ ID NO:22] (Barash, et al., 2002, Biochem and Biophys Res. Comm., 294:835-842).

Vectors encoding a TCR α chain comprised the insert: 5′-EcoRI restriction site-signal peptide-α chain-TGA stop-BamH1 restriction site-3′.

Vectors encoding a TCR β chain comprised the insert: 5′-EcoRI restriction site-signal peptide-β chain-TGA stop-BamH1 restriction site-3′.

Vectors encoding the IgG1 heavy chain comprised the insert: 5′-EcoR1 restriction site-signal peptide-IgG1 CH2 domain and CH3 domain terminating at G446 (EU numbering)-TGA stop-BamH1 restriction site-3′.

Vectors encoding the IgG1 light chain comprised the insert: 5′-EcoRI restriction site-signal peptide-IgG1 light chain-TGA stop-BamH1 restriction site-3′.

TCR chains fused to the N-terminus of an IgG1 Fc were linked to the upper hinge of the IgG1 heavy chain at position E216 (EU numbering) and included the CH2 domain and the CH3 domain terminating at position G446 (EU numbering) followed by a TGA stop codon and a BamH1 restriction site.

In the case of TCR chains fused to the C-terminus of an IgG1 Fc, the TCR chain was followed by a short sequence of the upper hinge starting at E216 and terminating at T225 (EU numbering).

All expression vectors were sequenced to confirm correct reading frame and sequence of the coding DNA.

### Example 2: Comparison of Expression of TCR Fusion Proteins in Different Formats

TCR fusion proteins in the formats shown in Table 2.1 were produced using a mammalian transient transfection protocol. The formats tested included TCR domains fused in different orientations to determine whether orientation affected production of the fusion proteins. A modified anti-gp100 TCR domain and an anti-CD3 scFv were used in the fusion proteins. All TCR fusion proteins which included both the TRAC and TRBC domains also contained the IC Disulfide (see Example 1).

The relevant TCRα, TCRβ, IgG1 heavy and scFv chains of the TCR fusion proteins were co-expressed in 2.5 mL cultures of Expi293F™ cells (Thermo Fisher, Waltham, MA) as described below.

Expi293™ cells were cultured at 37° C. in Expi293™ Expression Medium (Thermo Fisher, Waltham, MA) on an orbital shaker rotating at 125 rpm in a humidified atmosphere of 8% CO₂. A volume of 2.5 mL with a total cell count of 7.5×10⁷ cells was transfected with a total of 2.5 μg DNA. Prior to transfection, the DNA was diluted in 0.15 mL Opti-MEM™ I Reduced Serum Medium (Thermo Fisher, Waltham, MA) to provide a DNA transfection mix. 8 μL of ExpiFectamine™ 293 reagent (Thermo Fisher, Waltham, MA) were diluted in a volume of 0.15 mL Opti-MEM™ I Reduced Serum Medium and, after incubation for five minutes, the solution was combined with the DNA transfection mix to a total volume of 0.30 mL. After 10 to 20 minutes, the DNA-ExpiFectamine™293 reagent mixture was added to the cell culture. After incubation at 37° C. for 18-22 hours, 15 μL of ExpiFectamine™ 293 Enhancer 1 and 0.15 mL of ExpiFectamine™ 293 Enhancer 2 (Thermo Fisher, Waltham, MA) were added to each culture. Cells were incubated for five days and supernatants were harvested.

The protein levels in the supernatants were quantified using an Octet™ RED96 (ForteBio, Fremont, CA) with a Protein A tip. 200 μL of each culture supernatant were transferred into a 96 well plate. Samples were measured 3 times for 120 seconds. After each read, the tip was regenerated for 5 seconds in 100 mM glycine, pH 1.5, followed by a 5-second neutralization in PBS. Measurements were compared against a standard curve to obtain the protein concentration for each sample. The results are shown in Table 2.1.

TABLE 2.1
Expression Titers for TCR Fusion Proteins

| Variant # | Description¹ | Titer (mg/L)² |
|---|---|---|
| v21203 | One-armed scFv | 33.1 |
| v21203-B | Dual scFv (no Fc) | 14.9 |
| v21204 | One-armed TCR alpha fusion | 1.5 |
| v21204-B | Dual TCR alpha fusion (no Fc) | 0 |
| v21205 | Chimeric bispecific | 4.4 |
| v21207 | TCR bispecific alpha fusion | 5.9 |
| v21230 | One-armed TCR beta fusion | 11 |
| v21230-B | Dual TCR beta fusion (no Fc) | 1.1 |
| v21232 | TCR bispecific beta fusion | 11.6 |
| v21231 | TCR dual fusion | 8.4 |

¹See Fig. 1
²From a 3mL expression volume

As can be seen from Table 2.1, the TCR fusion proteins linked to the Fe through the beta-chain (v21230 and v21232) showed the highest expression titers. Expression titers for the TCR fusion proteins overall were lower than those for the scFv constructs. Those variants listed in Table 2.1 including a “-B” suffix did not include an Fc domain and, therefore, consisted of just 2 TCRs or 2 scFvs. In all cases, these homodimer variants showed lower expression titers.

### Example 3: Identification of Potential Stabilizing Mutations in the TCR Constant Domains

A structure and computational guided approach was employed to produce a library of mutation designs in the TRAC and TRBC domains that could potentially improve the thermal and/or colloidal stability of the TCR. These mutations where selected based upon improving surface properties, interface interactions and internal packing as described in more detail below.

Design of the mutations was focused on stabilizing the TRAC and TRBC domains as these contain very little variation between TCRs. The TRAC domain is identical among all human derived TCRs and the TRBC domain has only two different allotypes. The TRBC sequences differ in only 3 residues between TRBC1 and TRBC2: position TRBC/1.4 is Lys in TRBC1 and Gln in TRBC2, position TRBC/1.3 is Gln in TRBC1 and Lys in TRBC2, and position TRBC/29 is Tyr in TRBC1 and Phe in TRBC2.

An in silico TCR model was prepared using H27-14 TCR (Protein Data Bank reference: pdb:3VXS) as a framework.

### Identification of Hydrophobic Patch Mutations

Hydrophobic patches were identified using the Protein Patches tool in Molecular Operating Environment (Version 2019.01; Chemical Computing Group, Montreal, QC). All non-polar residues located in the TRAC or TRBC domains in hydrophobic patches having an area greater than 50 Å with surface exposed sidechains were flagged.

### Identification of Stabilizing Point Mutations

The crystal structure residues of H27-14 were analyzed via in silico mutagenesis and packing/modeling. In silico mutations were made at every position in the TRAC and TRBC domains of the TCR. Each residue was substituted by all possible amino acids, except proline and cysteine. These analyses resulted in the identification of a list of hotspot positions for engineering preferential alpha-beta pairing. Mutations used to improve stability were classified into different categories:
1) Cavity—located inside the core of a domain and improves hydrophobic packing
2) Interface—located at the interface of two domains and improves interfacial interactions
3) Patch—located at the surface and reduces a surface exposed hydrophobic patch.

Cavity, Interface and Patch mutations were ranked based on improvements in force field metrics. Patch mutations were further ranked based on the reduction of hydrophobic patches.

### Identification of Loop Truncation Mutants

Truncation of the TRBC FG loop in Fab/TCR chimeras has previously been reported to improve expression of the chimera (Wu, et al., 2015, MABS, 7:364-376). Different loop truncations of the FG loop were investigated to determine whether these could improve the expression in full TCRs. As the TRBC DE loop is shorter in many non-human TCRs, truncation of the human TRBC DE loop was also investigated, as well as truncation with addition of a Gly-Asn beta turn motif.

### Ranking of Stabilization Mutants

Potential stabilizing mutations and designs at the identified hotspot positions as well as positions neighboring hotspots of interest in the 3D crystal structure were simulated and identified via in silico mutagenesis and packing/modeling, and scored on the basis of a number of factors including steric and electrostatic improvements. Steric improvements were modeled and also computed on the basis of energy factors such as van der Waals packing, cavitation effects and close contact of hydrophobic groups. Similarly, electrostatic interaction energies were modeled and evaluated on the basis of coulomb interactions between charges, hydrogen bonds and desolvation effects. Potential stabilizing mutations identified through this analysis are listed in Table 3.1.

TABLE 3.1
Potential Stabilizing Mutations Identified in silico

| Position | Mutation | Type |
|---|---|---|
| TRAC/3 | ALA->THR | Cavity |
| TRAC/3 | ALA->SER | Cavity |
| TRAC/4 | VAL->ILE | Cavity |
| TRAC/26 | THR->ILE | Cavity |
| TRAC/39 | VAL->ILE | Cavity |
| TRAC/85.1 | SER-> VAL | Interface |
| TRAC/85 | ALA->THR | Cavity |
| TRAC/85 | ALA->VAL | Cavity |
| TRAC/87 | ALA->VAL | Cavity |
| TRAC/107 | ALA->SER | Cavity |
| TRBC/6 | VAL->LEU | Cavity |
| TRBC/6 | VAL->ILE | Cavity |
| TRBC/20 | THR->ILE | Interface |
| TRBC/20 | THR->VAL | Interface |
| TRBC/25 | ALA->VAL | Cavity |
| TRBC/36 | HIS->PHE | Interface |
| TRBC/36 | HIS->GLN | Interface |
| TRBC/37 | VAL->ILE | Cavity |
| TRBC/85.1 | CYS->VAL | Interface |
| TRBC/85.1 | CYS->MET | Interface |
| TRBC/86 | SER->VAL | Cavity |
| TRBC/86 | SER->THR | Cavity |
| TRBC/106 | VAL->MET | Cavity |
| TRBC/106 | VAL->ILE | Cavity |
| TRBC/45.3 | VAL->THR | Patch |
| TRBC/91 | VAL->GLN | Patch |
| TRBC/95 | PHE->TYR | Patch |
| TRAC/105 | ALA->SER | Patch |
| TRAC/115 | ILE->THR | Patch |
| TRAC/120 | GLU->TYR | Patch |
| TRBC/109-114 | Replacement of amino | Loop Trimming - FG |
| | acids at positions | |
| | 109-114 with GLY-ASN | |
| TRBC/109-114 | Replacement of amino | Loop Trimming - FG |
| | acids at positions 109-114 | |
| | with LYS-PRO-SER-ASN | |
| TRBC/84.5-85.6 | Deletion of amino acids | Loop Trimming - DE |
| | at positions 84.5-85.6 | |
| TRBC/84.4-85.4 | Replacement of amino | Loop Trimming - DE |
| | acids at positions | |
| | 84.4-85.4 with GLY-ASN | |

### Example 4: Expression and Characterization of TCR Fusion Proteins Comprising Potential Stabilizing Mutations

To determine the effect of the potential stabilizing mutations identified in Example 3 on TCR stability, TCR fusion proteins comprising the mutations were constructed in a one-armed (GA) format with the β chain fused to the Fc as described in Example 1. The TCR fusion proteins were expressed and tested in vitro for stability to identify the mutations that provided the greatest improvement in expression, thermal stability and/or colloidal stability. A modified anti-gp100 TCR domain was used in the fusion proteins. All TCR fusion proteins contained the IC Disulfide (see Example 1).

HEK293-6E cells at a density of 1.5-2.2×10⁶ cells/ml were cultured at 37° C. in FreeStyle™ F17 medium (GIBCO Cat #A13835-01) supplemented with G418 sulfate (Wisent Bioproducts Cat #400-130-IG1), 4 mM glutamine and 0.1% Pluronic™ F-68 (GIIBCO Cat #24040-032). A total of 1 ug DNA (50% variant DNA, 5% GFP, 15% AKT, 30% ssDNA) per ml of HEK293-6E cells was transfected at a ratio of 40:30:30 for alpha chain, beta chain-Fc (A), and Fc (B) using PEI-max (Polysciences Cat #24765-2) at a DNA:PEI ratio of 1:2.5 and cells were incubated at 37° C. for 24 hours. Following incubation, 0.5 mM valproic acid (final concentration) and 0.5% w/v tryptone N1 (final concentration) were added to the cells. The cells were then transferred to 37° C. and incubated for 7 days prior to harvesting. Culture media was harvested by centrifugation and vacuum filtered using a Stericup® 0.22 μM filter (Millipore Cat #SCGPU05RE). Samples were initially tested for expression in 0.8 mL volume. Samples which showed protein bands on an SDS-PAGE gel were scaled up to 250-500 mL cultures.

To purify the TCR fusion protein, cells were first removed from the supernatants by centrifuging at 1000rcf for 15 minutes. Protein A Gravitrap™ columns were prepared by equilibration using 10 ml PBS, followed by application of protein supernatant in batches of 10 ml. Once all the supernatant had flowed through the column, the column was washed with 2×10 ml PBS. TCR fusion protein was eluted by the addition of 3 ml 0.1M glycine-HCl, pH 2.7. The eluted TCR fusion protein was then neutralized using 1M Tris-HCl, pH 9. Protein yield was quantitated based on absorbance at 280 nm (A280 nm) (in instances where precipitation was present upon sample neutralization, samples were centrifuged briefly prior to A280 nm measurements).

Homogeneity of the TCR fusion proteins was assessed by UPLC-SEC. UPLC-SEC was performed using a Waters ACQUITY BEH200 SEC column (2.5 mL, 4.6×150 mm, stainless steel, 1.7 m particles) (Waters Ltd, Mississauga, ON) set to 30° C. and mounted on a Waters ACQUITY UPLC H-Class Bio system with a Photodiode Array (PDA) detector. Run times were 7 min with a total volume per injection of 2.8 mL using a running buffer of Dulbecco's phosphate-buffered saline (DPBS) or DPBS with 0.02% Tween 20, pH 7.4, at 0.4 ml/min. Elution was monitored by UV absorbance in the range 210-500 nm and chromatograms were extracted at 280 nm. Peak integration was performed using Empower 3 software (Waters Ltd, Mississauga, ON).

Samples with acceptable homogeneity (>95%) were buffer exchanged into DPBS and aseptically filtered post protein-A purification. Samples with low homogeneity were subjected to SEC purification as follows. Samples were loaded onto a Superdex® 200 10/30 Increase column (GE Healthcare Life Sciences, Marlborough, MA) on an Akta™ Avant 25 Chromatography System (GE Healthcare Life Sciences, Marlborough, MA) in DBPS with a flow rate of 0.5 mL/min. Fractions of eluted protein were collected based on absorbance at 280 nm and the fractions were assessed by non-reducing and reducing High Throughput Protein Express assay using Caliper LabChip® GXII (Perkin Elmer, Waltham, MA). Procedures were carried out according to HT Protein Express LabChip® User Guide version2 and LabChip GXII User Manual, with the following modifications. TCR fusion protein samples at either 2 μl or 5 μl (concentration range 5-2000 ng/μl) were added to separate wells in 96 well plates (BioRad, Hercules, CA) along with 7 μl of HT Protein Express Sample Buffer (Perkin Elmer Cat #760328). TCR fusion protein samples were then denatured at 70° C. for 15 mins. The LabChip® instrument was operated using the HT Protein Express Chip (Perkin Elmer, Waltham, MA) and the Ab-200 assay setting.

Thermal stability of the TCR fusion proteins was determined by differential scanning calorimetry (DSC). Each purified TCR fusion protein was diluted to 1 mg/mL in PBS. A total of 950 μL was used for DSC analysis with a NanoDSC (TA Instruments, New Castle, DE). At the start of each DSC run, a buffer blank injection was performed to stabilize the baseline, and a buffer injection was placed before each TCR fusion protein injection for referencing. Each sample was scanned from 25° C. to 95° C. at a rate of 60° C./hr and 60 psi nitrogen pressure. The resulting thermograms were referenced and analyzed using NanoAnalyze (TA Instruments, New Castle, DE). Each peak on the DSC thermogram corresponds to a thermal transition. There are three expected thermal transitions: TCR, CH2 (˜71° C.) and CH3 (˜80° C.). The transition of the TCR includes the TRAV-TRBV and TRAC-TRBC interfaces.

The yield, homogeneity and Tm determined for each of the TCR fusion proteins are shown in Table 4.1. Note that the DNA ratio for alpha chain, beta chain-Fc (A), and Fc (B) used in the transfection step was not optimized which impacted the % correct species observed.

TABLE 4.1
Expression and Characterization of TCR Fusion Proteins
Comprising Potential Stabilizing Mutations

| | | | | HMW | Correct | TCR |
|---|---|---|---|---|---|---|
| | | Mutation | Yield | Species | Species | Tm |
| Variant # | Mutation¹ | Type | (mg/L) | (%)² | (%)² | (° C.) |
| v21230 | None | Control | 17.66 | 37.4 | 56.8 | 53.7 |
| v22703 | TRAC/3.ALA−>THR | Cavity | 18.19 | 39.3 | 29.4 | 50.7 |
| v22704 | TRAC/3.ALA−>SER | Cavity | 19.79 | 36.6 | 36.3 | 51.5 |
| v22705 | TRAC/4.VAL−>ILE | Cavity | 22.05 | 32.6 | 43.9 | 54.2 |
| v22706 | TRAC/26.THR−>ILE | Cavity | 19.72 | 39.6 | 30.9 | 56.7 |
| v22707 | TRAC/39.VAL−>ILE | Cavity | 23.59 | 29.9 | 48.4 | 56 |
| v22836 | TRAC/85.1.SER−> VAL | Interface | 16.28 | 59.8 | 21.4 | 51.7 |
| v22708 | TRAC/85.ALA−>THR | Cavity | No Ex.³ | — | — | — |
| v22709 | TRAC/85.ALA−>VAL | Cavity | 26.55 | 20.2 | 55.9 | 56.7 |
| v22710 | TRAC/87.ALA−>VAL | Cavity | No Ex. | — | — | — |
| v22711 | TRAC/107.ALA−>SER | Cavity | No Ex. | — | — | — |
| v22712 | TRBC/6.VAL−>LEU | Cavity | 33.37 | 25.4 | 62.2 | 54.6 |
| v28881 | TRBC/6.VAL−>ILE⁴ | Cavity | 31.2 | 10.5 | 89.5 | 56.4 |
| v22713 | TRBC/20.THR−>ILE | Interface | No Ex. | — | — | — |
| v22714 | TRBC/20.THR−>VAL | Interface | No Ex. | — | — | — |
| v22715 | TRBC/25.ALA−>VAL | Cavity | 16 | 44.4 | 29.8 | 52.8 |
| v22716 | TRBC/36.HIS−>PHE | Interface | 18.66 | 32.9 | 40 | 55.3 |
| v22717 | TRBC/36.HIS−>GLN | Interface | 13.94 | 45 | 29 | 51.9 |
| v22718 | TRBC/37.VAL−>ILE | Cavity | 17.97 | 37.2 | 38.2 | 53.6 |
| v22719 | TRBC/85.1.CYS−>VAL | Interface | 19.28 | 44.8 | 27.8 | 51.1 |
| v22720 | TRBC/85.1.CYS−>MET | Interface | 25.64 | 29.9 | 47 | 51.6 |
| v22721 | TRBC/86.SER−>VAL | Cavity | No Ex. | — | — | — |
| v22722 | TRBC/86.SER−>THR | Cavity | 23.66 | 34.7 | 46.9 | 53.9 |
| v22723 | TRBC/106.VAL−>MET | Cavity | No Ex. | — | — | — |
| v22724 | TRBC/106.VAL−>ILE | Cavity | 16.08 | 57.6 | 14.3 | —⁵ |
| v22837 | TRBC/45.3.VAL−>THR | Patch | 27.35 | 20.1 | 61 | 54 |
| v22838 | TRBC/91.VAL−>GLN | Patch | No Ex. | — | — | — |
| v22839 | TRBC/95.PHE−>TYR | Patch | 19.44 | 50.7 | 21.6 | 53.7 |
| v22840 | TRAC/105.ALA−>SER | Patch | 26.34 | 24.8 | 50.6 | 54 |
| v22841 | TRAC/115.ILE−>THR | Patch | No Ex. | — | — | — |
| v22842 | TRAC/120.PHE−>TYR | Patch | 16.64 | 52.6 | 46.1 | 54.1 |
| v22745 | TRBC/109-114−>GLY- | Loop | No Ex. | — | — | — |
| | ASN⁶ | Trimming - | | | | |
| | | FG | | | | |
| v22746 | TRBC/109-114−>LYS- | Loop | No Ex. | — | — | — |
| | PRO-SER-ASN⁷ | Trimming - | | | | |
| | | FG | | | | |
| v22772 | TRBC/84.5-85.6−>-⁸ | Loop | 21.2 | 27.6 | 50.5 | 54.6 |
| | | Trimming - | | | | |
| | | DE | | | | |
| v22748 | TRBC/84.4-85.4−>GLY- | Loop | 31.1 | 23 | 62 | 53 |
| | ASN⁹ | Trimming - | | | | |
| | | DE | | | | |

¹All variants included the IC Disulfide. Listed mutations are in addition to this disulfide.
²As determined by UPLC-SEC. Except where noted, the variants were expressed at 37° C. which resulted in higher amounts of HMW species (see Example 5).
³No Ex. = no expression
⁴This variant was expressed at 32° C., which was shown to reduce the amount of HMW species present in TCR preparations (see Example 5)
⁵Unable to determine Tm by DSC
⁶109-114−>GLY-ASN notation indicates replacement of amino acids at positions 109-114 with GLY-ASN
⁷109-114−>LYS-PRO-SER-ASN notation indicates replacement of amino acids at positions 109-114 with LYS-PRO-SER-ASN
⁸84.5-85.6−>- notation indicates deletion of amino acids at positions 84.5-85.6
⁹84.4-85.4−>GLY-ASN notation indicates replacement of amino acids at positions 84.4-85.4 with GLY-ASN.

Variants showing decreased high molecular weight (HMW) species (aggregation) or increased Tm compared to the control (v21230) were considered stabilized. As can be seen from Table 4.1, the TCR fusion proteins v22705, v22707, v22709, v22712, v22716, v22722, v22837, v22840 and v22772 showed both decreased HMW species and increased Tm when compared to the v21230 control. TCR fusion protein v28881 also showed decreased HMW species and increased Tm when compared to the v21230 control, however, this protein was expressed at 32° C., so was expected to show decreased HMW species. Several of the TCR fusion proteins showed reduced HMW species, with the greatest reduction in HMW species being observed for v22709 and v22837 (a reduction from 37.4% to 20.2% and 20.1%, respectively). Of the TCR fusion proteins that showed an improvement in Tm, the greatest increase was observed for v22706, v22709 and v28881. Both v22706 and v22709 showed an increase in Tm of 3° C. (from 53.7° C. to 56.7° C.), although v22706 did not show decreased HMW species, whereas v28881 showed an increase in Tm of 2.7° C. (from 53.7° C. to 56.4° C.).

As all the TCR fusion proteins contain identical sequences except for the point mutations described in Table 4.1, the observed improvements in homogeneity and/or Tm can be attributed to these point mutations.

### Example 5: Effect of Temperature and Cell Line on Expression of TCR Fusion Protein

The TCR fusion protein v21232 was produced under different expression conditions as described below in order to identify the conditions which resulted in the highest yields and lowest HMW species (aggregation).

A TCR bispecific beta fusion TCR fusion protein comprising an anti-gp100 TCR and an anti-CD3 scFv (v21232; see Example 2) was expressed in 2.5 mL of Expi293F™ cells (Thermo Fisher, Waltham, MA) or ExpiCHO™ cells (Thermo Fisher, Waltham, MA) with a H1:H2:L1 DNA ratio of 30:30:40.

For expression in Expi293F™ cells, cultures were prepared as described in Example 2 except a DNA ratio of 40:40:20 for alpha chain, beta chain-Fc(A), and Fc (B) was employed in the transfection step. After incubation at 37° C. for 18-22 hours, 15 μL of ExpiFectamine™ 293 Enhancer 1 and 0.25 mL of ExpiFectamine™ 293 Enhancer 2 (Thermo Fisher, Waltham, MA) were added to each culture. Cultures were then transferred to 37° C. or 32° C. for the remaining time of incubation prior to supernatant harvest.

For expression in ExpiCHO™ cells, cells were cultured at 37° C. in ExpiCHO™ expression medium (Thermo Fisher, Waltham, MA) on an orbital shaker rotating at 125 rpm in a humidified atmosphere of 8% CO₂. A volume of 2.5 ml of culture with a total cell count of 1.5×10⁸ cells was transfected with a total of 2 μg DNA with a DNA ratio of 40:40:20 for alpha chain, beta chain-Fc(A), and Fe (B). Prior to transfection, the DNA was diluted in 0.1 mL OptiPRO™ SFM (Thermo Fisher, Waltham, MA) to provide a DNA transfection mix. 8 μL of ExpiFectamine™ CHO reagent (Thermo Fisher, Waltham, MA) were diluted in a volume of 92 L OptiPRO™ SFM and, after incubation for one to five minutes, combined with the DNA transfection mix to a total volume of 0.2 mL. After one to five minutes, the DNA-ExpiFectamine™ CHO Reagent mixture was added to the cell culture. After incubation at 37° C. for 18-22 hours, 15 L of ExpiCHO™ Enhancer and 0.6 mL of ExpiCHO™ Feed (Thermo Fisher, Waltham, MA) were added to each culture. Cultures were then transferred to 37° C. or 32° C. and incubated for seven days. Supernatants were then harvested.

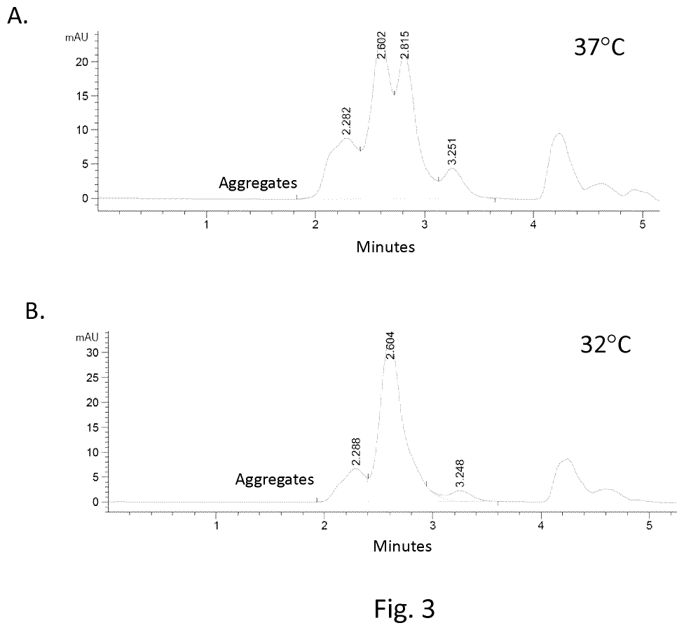

Titers of protein in the harvested supernatants were measured by Biolayer Interferometry as described in Example 2. Measured titers in Expi293F™ cells were significantly higher than in ExpiCHO™ cells (see Table 5.1), so the samples expressed in Expi293F™ cells were purified by protein A as described in Example 4. Homogeneity was determined by UPLC-SEC as described in Example 4. Peak area corresponding to the correct molecular weight was used to estimate the % of desired monodispersed species. The results are shown in Table 5.1 and FIG. 3.

TABLE 5.1
Effect of Temperature and Cell Line on Expression of v21232

| | Expression | | Desired | Estimated Total |
|---|---|---|---|---|
| | Temperature | Titer | Monodispersed | Desired Species |
| Cell Line | (° C.) | (mg/L) | Species¹ | (mg/L) |
| Expi293F ™ | 37 | 203 | 37.7% | 76.5 |
| ExpiCHO ™ | 37 | 28.3 | — | — |
| Expi293F ™ | 32 | 145 | 79.6% | 115.4 |
| ExpiCHO ™ | 32 | 49.1 | — | — |

¹As determined by UPLC-SEC.

As can be seen from Table 5.1, expression of variant v21232 in Expi293F™ cells at a reduced temperature of 32° C. produced the greatest amount of the desired protein. While higher temperature (37° C.) increased the total amount of protein produced, a significant portion of the protein formed high molecular weight species. As can be seen from Table 5.1 and FIG. 3, the TCR fusion protein produced at 32° C. contained a greatly reduced amount of high molecular species and an increased amount of desired protein. The Expi293F™ cells consistently produced more protein than the ExpiCHO™ cells. Accordingly, only samples produced from Expi293F™ were purified and characterized by UPLC-SEC.

### Example 6: Addition of a Hinge-Like Disulfide Bond to Stabilize TCR Fusion Proteins

To improve the stability of the TCR fusion proteins, fusion proteins comprising a disulfide bond that mimics the light chain-upper hinge disulfide bond found in IgG1 antibodies were prepared as follows.

The natural TCR sequence includes a C-terminal disulfide bond located in the linker region between the constant domains and the transmembrane domains. A potential stability enhancing modification was designed that introduced a disulfide bond between the natural C-terminal cysteine on the TCR alpha chain (referred to as position TRAC/128.CYS, which is typically omitted from soluble TCR constructs) and the natural cysteine located in the upper hinge region of the Fc (220.CYS (EU numbering)) in the beta-Fc fusion chain. The disulfide bond formed between these two cysteines (i.e. TRAC/128.CYS and hinge 220.CYS) is referred to herein as the “TRAC-Hinge Disulfide.”

The TRAC-Hinge Disulfide was initially tested in two TCR fusion proteins: an anti-gp100 TCR fused to an Fc and an anti-NY-ESO1 1G4-HA (high affinity) TCR fused to an Fc. Both TCR fusion proteins also contained the IC Disulfide. The TCR fusion proteins were constructed in a one-armed format with the beta chain fused to the Fc as described in Example 1. All sequences were preceded by the signal peptide: MRPTWAWWLFLVLLLALWAPARG [SEQ ID NO:22]. Vector inserts were prepared and cloned into the pTT5 vector for expression as described in Example 1.

The TCR fusion proteins were initially expressed in 0.8 mL of HEK293-6E cells as described in Example 4 and expression was confirmed by SDS-PAGE. The fusion protein was subsequently expressed in 250 mL of HEK293-6E cells as described in Example 4 but with a DNA ratio of 40:40:20 for alpha chain, beta chain-Fc(A), and Fc (B). After the initial 18-22 hour incubation at 37° C., the cells were incubated at 32° C. and subsequently purified and characterized by UPLC-SEC as described in Example 4.

Thermal stability of the TCR fusion proteins was determined by differential scanning calorimetry (DSC). Each purified TCR fusion protein was diluted to 0.4 mg/mL in PBS. A total of 400 μL was used for DSC analysis with a MicroCal™ VP-Capillary DSC (GE Healthcare Life Sciences, Chicago, IL). At the start of each DSC run, 5 buffer blank injections were performed to stabilize the baseline, and a buffer injection was placed before each TCR fusion protein injection for referencing. Each sample was scanned from 20° C. to 100° C. at a rate of 60° C./hr with low feedback, 8 sec filter, 5 min preTstat, and 70 psi nitrogen pressure. The resulting thermograms were referenced and analyzed using Origin 7 software (OriginLab Corporation, Northampton, MA). Each peak on the DSC thermogram corresponds to a thermal transition. There are three expected thermal transitions: TCR, CH2 (˜71° C.) and CH3 (˜80° C.). The transition of the TCR includes the TRAV-TRBV and TRAC-TRBC interfaces.

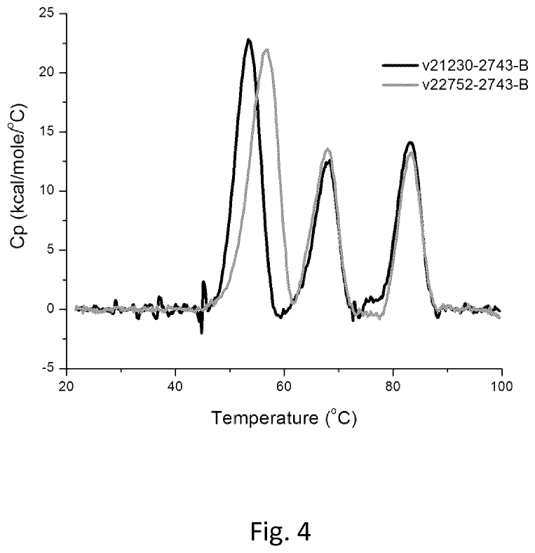

The results are shown in Table 6.1 and FIG. 4.

TABLE 6.1
Expression and Characterization of TCR Fusion
Proteins Comprising the TRAC-Hinge Disulfide

| | | TRAC- | | HMW | Correct | LMW | TCR |
|---|---|---|---|---|---|---|---|
| | | Hinge | Yield | Species | Species | Species | Tm |
| Variant # | TCR | Disulfide | (mg/L) | (%)¹ | (%)¹ | (%) | (° C.)² |
| v21230 | Anti-gp100 | No | 22.8 | 7.7 | 92.7 | 0.7 | 53.4 |
| v22752 | Anti-gp100 | Yes | 18.2 | 10.7 | 88.8 | 0.3 | 56.9 |
| v30930 | Anti-NY- | No | 19.2 | 3.9 | 60 | 36.1 | 62.2 |
| | ESO1 1G4- | | | | | | |
| | HA | | | | | | |
| v31185 | Anti-NY- | Yes | 17.6 | 5.4 | 89.4 | 5.2 | 64.4 |
| | ESO1 1G4- | | | | | | |
| | HA | | | | | | |

¹As determined by UPLC-SEC.
²As determined by DSC.

As can be seen in Table 6.1 and FIG. 4, the anti-gp100 TCR fusion protein comprising the TRAC-Hinge Disulfide showed an increase in Tm of >3° C. compared to the same fusion protein lacking this additional disulfide bond (compare variants v21230 and v22752). Similarly, the anti-NY-ESO1 1G4-HA TCR fusion protein comprising the TRAC-Hinge Disulfide showed an increase in Tm of ˜2° C. compared to the same fusion protein lacking this additional disulfide (compare variants v30930 and v31185). The anti-NY-ESO1 1G4-HA TCR fusion protein comprising the TRAC-Hinge Disulfide also showed an increase of 29.4% in correct species compared to the TCR fusion protein lacking the TRAC-Hinge Disulfide. These results show that the TRAC-Hinge Disulfide is able to stabilize TCR fusion proteins and is also transferable to different TCR fusion proteins.

### Example 7: Identification of Additional Disulfide Bonds for Stabilization of TCR Fusion Proteins

To improve the stability of TCR fusion proteins, mutations for inclusion in the TRAC and/or TRBC domains in order to introduce new disulfide bonds were identified by in silico modeling.

A TCR model as described in Example 3 was used to identify positions to introduce novel disulfide bonds. The Cβ-Cβ and Cα-Cα pairwise distance for every non-cysteine residue located in the TRAC and TRBC domains with every other residue in both the alpha and beta chains was calculated. Residue pairs with Cβ-Cβ distances <5 Å or Cα-Cα distances <7 Å that were separated in sequence space by more than five residues were considered positive hits for potential disulfide bonds. All flagged residue pairs were visually inspected to confirm that the two residues were in the correct orientation to generate a disulfide bond. Potential disulfide bonds which passed the above criteria were generated as a model in silico.

The lowest energy conformation of each disulfide bond was used to rank the models using the following criteria:
the disulfide dihedral angle energy function described by Katz & Kossiakoff, 1986, J Biol Chem, 261(33):15480-15485
the degree of structural perturbation as measured by backbone and sidechain root-mean-square deviation (RMSD)
the number of steric clashes in the structure, and
the changes in either folding energy or interaction energy upon the introduction of intrachain and interchain disulfide bonds, respectively.

The top hits which were further investigated experimentally are listed in Table 7.1.

TABLE 7.1
Top Disulfide Bonds Identified in silico

| | Cα_Cα | Cβ_Cβ | |
|---|---|---|---|
| | Distance | Distance | |
| Residue Pair | (Å) | (Å) | Interchain |
| TRAC/122.PRO_TRBC/12.ALA | 6.20 | 3.47 | Yes |
| TRAC/84.2.LEU_TRBC/79.SER | 4.66 | 3.70 | Yes |
| TRBC/36.HIS_TRBC/109.TYR | 5.41 | 3.72 | No |
| TRAC/3.ALA_TRAC/120.PHE | 4.18 | 3.72 | No |
| TRBC/9.PRO_TRBC/19.ALA | 5.15 | 3.74 | No |
| TRAC/10.SER_TRBC/121.ALA | 5.52 | 3.75 | Yes |
| TRAC/7.LEU_TRBC/7.PHE | 5.39 | 3.84 | Yes |
| TRAC/84.THR_TRBC/79.SER | 6.02 | 3.94 | Yes |
| TRAC/5.TYR_TRBC/10.SER | 6.43 | 4.01 | Yes |
| TRAC/26.THR_TRAC/85.1.SER | 4.31 | 4.16 | No |
| TRAC/22.VAL_TRAC/88.TRP | 4.83 | 4.32 | No |
| TRAC/39.VAL_TRAC/85.ALA | 7.37 | 4.34 | No |
| TRBC/7.PHE_TRBC/22.VAL | 5.11 | 4.37 | No |
| TRBC/21.LEU_TRBC/89.LEU | 5.16 | 4.59 | No |
| TRAC/86.VAL_TRBC/86.SER | 7.51 | 4.67 | Yes |
| TRAC/84.1.THR_TRBC/86.SER | 7.39 | 4.76 | Yes |
| TRAC/10.SER_TRBC/6.VAL | 5.88 | 4.79 | Yes |
| TRAC/84.5.ARG_TRBC/45.5.SER | 5.88 | 4.81 | Yes |
| TRAC/84.4.MET_TRBC/77.GLY | 4.18 | N/A | Yes |
| TRAV/127.ARG_TRAC/21.SER | 6.15 | 3.85 | No |

### Example 8: Production and Characterization of TCR Fusion Proteins Comprising New Interchain Disulfide Bonds

The effect on stability of the interchain disulfide bonds identified in Example 7 was investigated in TCR fusion proteins in a one-armed format by analyzing the expression, thermal stability and colloidal stability of the TCR fusion proteins.

Each of the top-ranked interchain disulfide bonds identified in Example 7 (see Table 7.1) was introduced into an anti-gp100 TCR fused to an Fc. The TCR fusion proteins were constructed in a one-armed format with the beta chain fused to the Fc as described in Example 1. All sequences were preceded by the signal peptide: MRPTWAWWLFLVLLLALWAPARG [SEQ ID NO:22]. Vector inserts were prepared and cloned into the pTT5 vector for expression as described in Example 1.

The TCR fusion proteins were initially tested for expression in 0.8 mL of HEK293-6E cells as described in Example 4. Expression was confirmed by SDS-PAGE and variants with observable protein expression bands were subsequently expressed in 250 mL of HEK293-6E cells as described in Example 4 but with a DNA ratio of 40:40:20 for alpha chain, beta chain-Fc(A), and Fc (B). After the initial 18-22 hour incubation, the cells were incubated at either 32° C. or 37° C. and subsequently purified and characterized by UPLC-SEC as described in Example 4 and DSC as described in Example 6.

The results are shown in Table 8.1.

TABLE 8.1
Expression of TCR Fusion Proteins Comprising Interchain Disulfide Bonds

| | | | HMW | Desired | LMW | TCR |
|---|---|---|---|---|---|---|
| | Disulfide Bond | Expression | Species | Species | Species | Tm |
| Variant # | Position | (mg/L) | (%)¹ | (%)¹ | (%)¹ | (° C.)² |
| v21230 | TRAC/84-TRBC/79 | 17³ | 37.4³ | 56.8³ | 5.8³ | 53.7 |
| | (IC Disulfide) | | (7.6)⁴ | (91.7)⁴ | (0.7)⁴ | |
| v22726 | TRAC/7-TRBC/7 | No Ex⁵ | — | — | — | — |
| v22729 | TRAC/84.2_TRBC/79 | 16³ | 48.6³ | 23.5³ | 27.9³ | 49.2 |
| v22734 | TRAC/22_TRBC/7 | No Ex⁵ | — | — | — | — |
| v22735 | TRAC/84.4_TRBC/77 | No Ex⁵ | — | — | — | — |
| v22736 | TRAC/86_TRBC/86 | No Ex⁵ | — | — | — | — |
| v22737 | TRAC/84.5_TRBC/45.5 | No Ex⁵ | — | — | — | — |
| v22738 | TRAC/10_TRBC/121 | No Ex⁵ | — | — | — | — |
| v31093 | TRAC/122_TRBC/12 | 21⁴ | 34.3⁴ | 37.8⁴ | 27.9⁴ | 51.4 |

¹As determined by UPLC SEC
²As determined by DSC
³Expression at 37° C.
⁴Expression at 32° C.
⁵No expression at 37° C.

Two of the identified interchain disulfide bonds were observed to express and to stabilize the TCR fusion proteins: TRAC/84.2_TRBC/79 (v22729) and TRAC/122_TRBC/12 (v31093). Both variants had a lower Tm and higher HMW species than v21230 (IC Disulfide).

### Example 9: Production and Characterization of TCR Fusion Proteins Comprising New Intrachain Disulfide Bonds

The effect on stability of the intrachain disulfide bonds identified in Example 7 was investigated in TCR fusion proteins in a one-armed format by analyzing the expression, thermal stability and colloidal stability of the TCR fusion proteins.

Each of the top-ranked intrachain disulfide bonds identified in Example 7 (see Table 7.1) was introduced into an anti-gp100 TCR fused to an Fc. The TCR fusion proteins were constructed in a one-armed format with the beta chain fused to the Fc as described in Example 1. All variants contained the TRAC/84.2_TRBC/79 disulfide (see Example 8) and the TRAC-Hinge Disulfide. All sequences were preceded by the signal peptide: MRPTWAWWLFLVLLLALWAPARG [SEQ ID NO:22]. Vector inserts were prepared and cloned into the pTT5 vector for expression as described in Example 1.

The TCR fusion proteins were initially tested for expression in 0.8 mL of HEK293-6E cells as described in Example 4. Expression was confirmed by SDS-PAGE and variants with observable protein expression bands were subsequently expressed in 250 mL of HEK293-6E cells as described in Example 4 but with a DNA ratio of 40:40:20 for alpha chain, beta chain-Fc(A), and Fe (B). After the initial 18-22 hour incubation, the cells were incubated at 32° C. and subsequently purified and characterized by UPLC-SEC as described in Example 4 and DSC as described in Example 6.

The results are shown in Table 9.1.

TABLE 9.1
Expression of TCR Fusion Proteins Comprising Intrachain Disulfide Bonds

| | | | HMW | Desired | LMW | TCR |
|---|---|---|---|---|---|---|
| | | Expression | Species | Species | Species | Tm |
| Variant # | Disulfide Bond Position | (mg/L) | (%)¹ | (%)¹ | (%)¹ | (° C.)² |
| 28902 | Control³ | 15 | 6.3 | 92 | 1.7 | 52.5 |
| 31084 | TRAC/3.ALA_TRAC/120.PHE | No Ex⁴ | | | | |
| 31085 | TRAC/39.VAL_TRAC/85.ALA | 19 | 19.9 | 80 | 0 | 57.8 |
| 31086 | TRAC/26.THR_TRAC/85.1.SER | 22 | 13.3 | 85.1 | 1.6 | 54.2 |
| 30933 | TRBC/9.PRO_TRBC/19.ALA | 18 | 15.6 | 77.1 | 7.3 | 52.5 |

¹As determined by UPLC-SEC
²As determined by DSC
³TRAC/84.2_TRBC/79 disulfide and TRAC-Hinge Disulfide alone
⁴No expression

As can be seen from Table 9.1, variant v31085, which comprises a combination of a disulfide at positions TRAC/39.VAL_TRAC/85.ALA with the TRAC/84.2.THR_TRBC/79.LEU interchain disulfide and TRAC-Hinge Disulfide showed an increase in Tm of ˜5° C.

Variant v31086, which comprises a combination of a disulfide at positions TRAC/26.THR_TRAC/85.1.SER with the TRAC/84.2_TRBC/79 disulfide (Example 8) and TRAC-Hinge Disulfide showed an increase in Tm of ˜2° C.

### Example 10: Binding of Stabilized TCR Fusion Proteins to Target Peptide-MHC Complex

TCR fusion proteins from Example 4 with a TCR Tm equal or greater than that of the parent TCR fusion protein as well as variants v22729 and v22730 (which include a new disulfide bond, see Example 8) were tested for binding to their target peptide-MHC complex by flow cytometry as follows.

T2 cells (ATCC CRL-1992) were cultured in RPMI-1640 10% FCS, 1% Penicillin-Streptomycin. Cells were then centrifuged, resuspended and mixed with 10 uM gp100 peptide (YLEPGPVTA [SEQ ID NO:24]). After incubation at 37° C. for two hours to allow peptide binding, cells were washed, resuspended in PBS 1% FCS and added to wells of a 96 V-well plate at 25 ul per well. TCR fusion proteins were prepared using a parallel plate using a 1:3 serial dilution starting from a 1:20 dilution of stock (range of stock concentrations 0.54-1.09 mg/ml) and extending for 11 samples (resulting in lowest concentrations of 3-8 pM). The cell plate was then spun down, supernatants were removed and the TCR fusion protein solution added. The plate was kept on ice for 30 minutes, followed by a single wash in PBS 1% FCS and resuspension in 1:200 anti-human IgG Alexa 647 conjugate (Jackson ImmunoResearch Laboratories, Inc., West Grove, PA). The plate was kept on ice for a further 30 minutes, followed by two washes with PBS 1% FCS. 100 ul of PBS 1% FCS was then added to the wells and the plate analyzed by flow cytometry using a BD Fortessa™ X-20 (BD Biosciences, San Jose, CA).

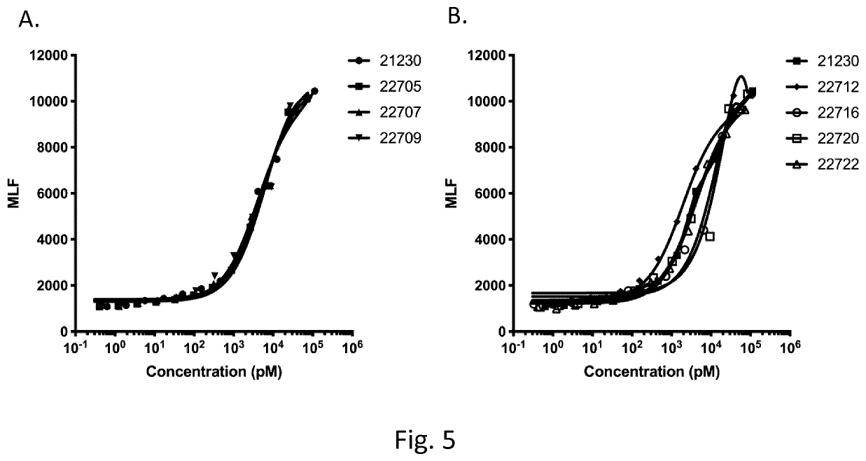

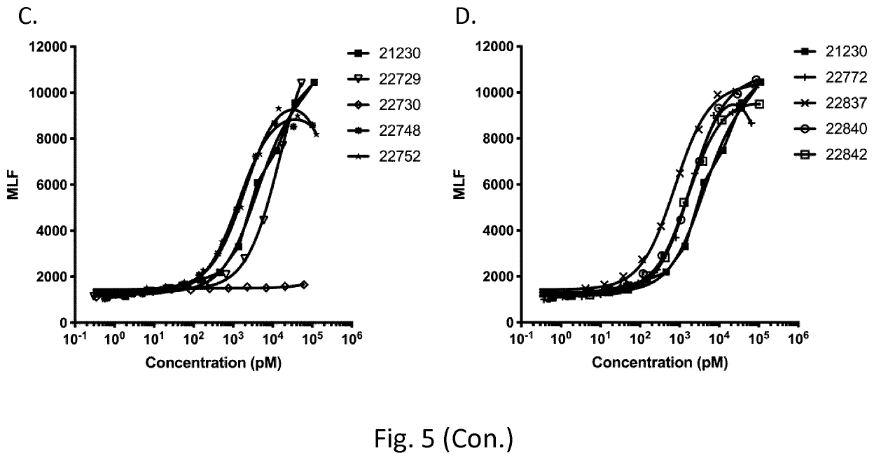

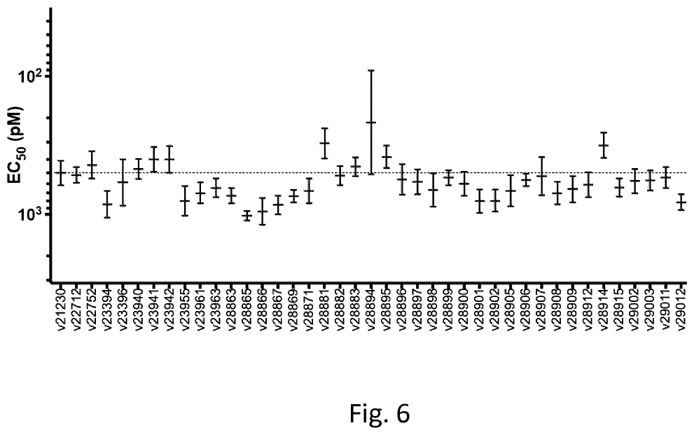

The results are shown in FIG. 5 and FIG. 6. As can be seen in FIG. 5, all TCR fusion proteins showed binding to the target peptide-MHC complex with the exception of variant v22730. However, the UPLC-SEC trace for variant v22730 suggested that most of the sample was not the desired species (see Example 8). The EC₅₀ for all other TCR fusion proteins was similar (see FIG. 6). These results demonstrate that the stabilizing mutations introduced into the TCR fusion proteins do not affect the binding of the protein to its target peptide-MHC complex.

### Example 11: TCR Fusion Proteins Comprising Combinations of Stabilizing Mutations

Examples 4 and 6-9 describe various mutations were identified that improved the thermal stability and/or colloidal stability of TCR fusion proteins. TCR fusion proteins comprising various combinations of these mutations were constructed to determine if the combined mutations could further improve thermal stability and/or colloidal stability of TCR fusion proteins.

The individual mutations identified in Examples 4 and 6-9 were combined as outlined in Table 11.1 and introduced into an anti-gp100 TCR fused to an Fc. The TCR fusion proteins were constructed in a one-armed format with the beta chain fused to the Fc as described in Example 1. All sequences were preceded by the signal peptide: MRPTWAWWLFLVLLLALWAPARG [SEQ ID NO:22]. Vector inserts were prepared and cloned into the pTT5 vector for expression as described in Example 1.

The TCR fusion proteins were initially tested for expression in 0.8 mL of HEK293-6E cells as described in Example 4. Expression was confirmed by SDS-PAGE and variants with observable protein expression bands were subsequently expressed in 250 mL of HEK293-6E cells as described in Example 4 but with a DNA ratio of 40:40:20 for alpha chain, beta chain-Fc(A), and Fc (B). After the initial 18-22 hour incubation, the cells were incubated at 32° C. for 5 days and subsequently purified and characterized by UPLC-SEC as described in Example 4 and DSC as described in Example 6. Binding of the TCR fusion proteins to the target peptide-MHC complex was confirmed using flow cytometry as described in Example 10.

The results are shown in Table 11.1. Also included in Table 12 are variants v33047 and v33048, which are discussed further in Example 12.

As can be seen in Table 11.1, TCR fusion proteins were successfully produced comprising various combinations of stabilizing mutations. The only unsuccessful combinations were those comprised by variants v23953, v28893, v28903 and v23398.

A number of stabilizing mutations were observed to improve the thermal stability when combined with the previously described IC Disulfide, with several variants that comprised combinations including the IC Disulfide demonstrating an increase in Tm of approximately 5° C. compared to the IC Disulfide alone. Specifically+:
v28897 (TRAC/85 Ala->Val, TRAC-Hinge Disulfide and IC Disulphide)
v29002 (TRAC/85 Ala->Val, TRBC/45.3 Val->Thr, TRAC-Hinge Disulfide and IC Disulphide)
v29003 (TRAC/85 Ala->Val, TRBC/6 Val->Leu, TRAC-Hinge Disulfide and IC Disulphide)
v28914 (TRAC/4 Val->Ile, TRAC/85 Ala->Val, TRBC/6 Val->Leu, TRAC-Hinge Disulfide and IC Disulphide)
v28915 (TRAC/4 Val->Ile, TRAC/85 Ala->Val, TRBC/6 Val->Leu, ΔTRBC/84.4-85.4->Gly-Asn, TRAC-Hinge Disulfide and IC Disulphide)
v29012 (TRAC/4 Val->Ile, TRAC/85 Ala->Val, TRAC/105 Ala->Ser, TRBC/6 Val->Leu, TRBC/36 His->Phe, TRBC/86 Ser->Thr, TRBC/45.3 Val->Thr, TRAC-Hinge Disulfide and IC Disulphide)

Combinations of stabilizing mutations with the TRAC/84.2_TRBC/79 interchain disulfide bond rather than the IC Disulfide also improved the thermal stability of TCR fusion proteins. As can be seen from Table 11.1, all TCR fusion proteins comprising the TRAC/84.2_TRBC/79 interchain disulfide bond and the TRAC-Hinge Disulfide in combination with 3 or more other stabilizing mutations showed a thermal stability greater than that of the IC Disulfide alone (see variants v29011, v31095, v31096, v31097, v31098, v31099, v31100, v31101, v31102, v31103, v31104 and v33048).

The TCR fusion proteins lacking the IC Disulfide that showed the greatest increase in thermal stability were:
v31099 (TRAC/39 Val->Ile, TRAC/85 Ala->Val, TRBC/36 His->Phe, TRAC/84.2 Leu->Cys_TRBC/79 Ser->Cys (disulfide) and TRAC-Hinge Disulfide)
v33048 (TRBC/6 Val->Ile, TRBC/36 His->Phe, TRAC/84.2 Leu->Cys_TRBC/79 Ser->Cys (disulfide), TRAC-Hinge Disulfide and TRAC/39 Val->Cys_TRAC/85 Ala->Cys (disulfide)).

TABLE 11.1
Characteristics of TCR Fusion Proteins Comprising Combinations of Stabilizing Mutations

| | Point Mutations | | | | | | | | | |
|---|---|---|---|---|---|---|---|---|---|---|
| | TRAC | TRAC | TRAC | TRAC | TRBC | TRBC | TRBC | TRBC | TRBC | Deletions |
| Variant | 4.VAL−> | 39.VAL−> | 85.ALA−> | 105.ALA−> | 6.VAL−> | 6.VAL−> | 36.HIS−> | 86.SER−> | 45.3.VAL−> | Δ_TRBC |
| Number | ILE | ILE | VAL | SER | LEU | ILE | PHE | THR | THR | 84.5-85.6 |
| v21230 | | | | | | | | | | |
| v28906 | x | | x | | | | | | x | |
| v23940 | x | | | | x | | | | | |
| v28908 | x | x | | | x | | | | | |
| v23941 | | x | | | x | | | | | |
| v28894 | | x | x | | | | | | | |
| v28895 | | x | | x | | | | | | |
| v23953 | | x | | | | | | | x | |
| v23961 | | x | | | | | | | | |
| v28907 | | | x | x | | | | | x | |
| v28909 | | | x | x | | | | | | |
| v23942 | | | x | | x | | | | | |
| v28896 | | | x | x | | | | | | |
| v22712 | | | | | x | | | | | |
| v28898 | | | | | x | | | | x | |
| v28899 | | | | | x | | | | | |
| v28912 | | | | | x | | | | x | |
| v28901 | | | | | | | x | | x | |
| v23955 | | | | x | | | | | x | |
| v23963 | | | | x | | | | | | |
| v22752 | | | | | | | | | | |
| v28893 | x | | | | | | | | | |
| v28914 | x | | x | | x | | | | | |
| v28915 | x | | x | | x | | | | | |
| v29012 | x | | x | x | x | | x | x | x | |
| v28897 | | | x | | | | | | | |
| v29002 | | | x | | | | | | x | |
| v29003 | | | x | | x | | | | | |
| v28900 | | | | | x | | | | | |
| v28903 | | | | | | | | | x | |
| v28905 | | | | | | | | | | |
| v23394 | | | | | x | | | | | |
| v23396 | | | | | x | | | x | | |
| v23398 | | | | | | | | x | | |
| v28902² | | | | | | | | | | |
| v31131 | | | | | | | | | | |
| v30934³ | | | | | | | | | | |
| v29011⁴ | x | | x | x | x | | x | x | x | |
| v31095 | | x | x | | x | | x | | | |
| v31096 | | x | x | | | x | x | | | |
| v31098 | | x | | | | x | x | | | |
| v31099 | | x | x | | | | x | | | |
| v31100 | | x | x | | | x | | | | |
| v31101 | | x | x | | | x | x | | x | |
| v31102 | | x | x | x | | x | x | | | |
| v31103 | | x | x | | | x | x | | | x |
| v31104 | | x | x | | | x | x | | | |
| v31097 | | | x | | | x | x | | | |
| v33047⁵ | | | | | | x | x | | | |
| v33048⁵ | | | | | | x | x | | | |

| | | Disulfide Bonds | | | | | | | |
|---|---|---|---|---|---|---|---|---|---|
| | Deletions | IC | | | | Measured | | | |
| | Δ_TRBC 84.4- | Disulfide | Disulfide | TRAC- | Disulfide | TCR | HMW | pA | Binding - |
| Variant | 85.4−>GLY- | (TRAC 84 - | TRAC 84.2 - | Hinge | TRAC 39 - | Tm | Species | Yield | EC50 |
| Number | ASN | TRBC 79) | TRBC 79 | Disulfide | TRAC 85 | (° C.) | (%) | (mg/L) | (pM) |
| v21230 | | x | | | | 53.4 | 7.7 | 17.7 | 500 |
| v28906 | | x | | | | 56.1 | 7.5 | 21.7 | 562 |
| v23940 | | x | | | | 54.7 | 12.8 | 26.4 | 468 |
| v28908 | | x | | | | 54.9 | 11.8 | 20.6 | 701 |
| v23941 | | x | | | | 55.1 | 13 | 27.8 | 399 |
| v28894 | | x | | | | 56 | 9.4 | 29.1 | 215 |
| v28895 | | x | | | | 55 | 10.3 | 28 | 382 |
| v23953 | | x | | | | N/D¹ | N/D | N/D | N/D |
| v23961 | x | x | | | | 54.6 | 11.4 | 22.7 | 700 |
| v28907 | | x | | | | 56 | 4.5 | 14.9 | 528 |
| v28909 | x | x | | | | 55.3 | 4.8 | 22.8 | 656 |
| v23942 | | x | | | | 56.1 | 10.3 | 23.2 | 399 |
| v28896 | | x | | | | 55.6 | 5.9 | 22.3 | 557 |
| v22712 | | x | | | | 54 | 10.7 | 33.4 | 517 |
| v28898 | | x | | | | 54.6 | 7.4 | 21.3 | 665 |
| v28899 | x | x | | | | 53.6 | 7.4 | 22.2 | 543 |
| v28912 | x | x | | | | 54.1 | 6 | 20.6 | 610 |
| v28901 | | x | | | | 54.8 | 10.6 | 14.7 | 802 |
| v23955 | | x | | | | 53.9 | 9.8 | 21.9 | 798 |
| v23963 | x | x | | | | 53.6 | 11 | 20.9 | 641 |
| v22752 | | x | | x | | 56.9 | 11.2 | 18.2 | 438 |
| v28893 | | x | | x | | N/D | N/D | N/D | N/D |
| v28914 | | x | | x | | 58.6 | 9.6 | 15 | 315 |
| v28915 | x | x | | x | | 58.1 | 10.3 | 17.5 | 640 |
| v29012 | x | x | | x | | 58.5 | 10.2 | 16.9 | 816 |
| v28897 | | x | | x | | 58.5 | 10.5 | 17.6 | 580 |
| v29002 | | x | | x | | 58.5 | 6 | 17.5 | 574 |
| v29003 | | x | | x | | 58.6 | 5.8 | 19.9 | 567 |
| v28900 | | x | | x | | 57.1 | 10.3 | 19.4 | 599 |
| v28903 | | x | | x | | N/D | N/D | N/D | N/D |
| v28905 | x | x | | x | | 56.6 | 7.9 | 17.9 | 674 |
| v23394 | | | x | | | 49.8 | 11.2 | 20.2 | 845 |
| v23396 | | | x | | | 49.8 | 11.8 | 19 | 586 |
| v23398 | | | x | | | N/D | N/D | N/D | N/D |
| v28902² | | | x | | | 52.5 | 6.3 | 14.8 | 796 |
| v31131 | | | x | x | | 53.3 | 10.4 | 24.1 | 520 |
| v30934³ | | | x | x | | 53.8 | 14.6 | 28.6 | 363 |
| v29011⁴ | x | | x | x | | 54.3 | 10.3 | 22.4 | 553 |
| v31095 | | | x | x | | 55.9 | 20 | 18.5 | 312 |
| v31096 | | | x | x | | 55.5 | 13.4 | 20.1 | 708 |
| v31098 | | | x | x | | 55 | 10.9 | 17.8 | 1225 |
| v31099 | | | x | x | | 56.2 | 17.7 | 17.8 | 802 |
| v31100 | | | x | x | | 54.6 | 12.9 | 23.4 | 3542 |
| v31101 | | | x | x | | 55.8 | 13.6 | 21.4 | 843 |
| v31102 | | | x | x | | 55.5 | 14.5 | 17 | 810 |
| v31103 | | | x | x | | 55.5 | 14.1 | 19 | 907 |
| v31104 | x | | x | x | | 54.8 | 16.2 | 17.9 | 543 |
| v31097 | | | x | x | | 55 | 9.45 | 24.1 | 913 |
| v33047⁵ | | | x | | x | 54.8 | 11.2 | 23 | —⁶ |
| v33048⁵ | | | x | x | x | 58.7 | 6.6 | 32.4 | —⁶ |

¹N/D = no data, expression level too low to purify
²This variant includes the mutations TRAC/1.5.GLN−>LYS and TRBC/97.GLN−>ASP as described in Example 1
³This variant is based on the TRBC1 constant framework (all other variants are based on the TRBC2 constant framework)
⁴Values provided are averaged from 3 different expressions of this variant
⁵See Example 12
⁶Not determined

### Example 12: TCR Fusion Proteins Comprising Alternative Disulfide Bonds to the TRAC-Hinge Disulfide

Example 11 describes various combinations of mutations capable of improving thermal stability and/or colloidal stability of the TCR fusion proteins in the absence of the IC Disulfide. All the stabilizing combinations identified included the TRAC Hinge Disulfide. As certain formats of TCR fusion protein may not accommodate the TRAC-Hinge Disulfide, combinations of mutations using alternative disulfide bonds were investigated to identify those capable of improving the thermal and/or colloidal stability of TCR fusion proteins.

TCR fusion proteins were constructed that comprised replacements for the TRAC-Hinge Disulfide that mimicked an IgG4 disulfide together with combinations of stabilizing point mutations. The replacement disulfide consisted of TRBC/11.CYS acting as an equivalent to the IgG4 CH1 cysteine and one of TRAC/124, 125, 126, 127 or 128 acting as an equivalent to the light-chain (LC) cysteine.

The disulfide introduced at positions TRAC/122.PRO_TRBC/12.ALA described in Example 8 is also located near the C-terminus of the TCR and was tested with combinations of stabilizing point mutations as another alternative to the TRAC-Hinge Disulfide.

Also tested were variants comprising the intrachain disulfide TRAC/39.VAL_TRAC/85.ALA (see Example 9) with combinations of stabilizing point mutations, both with and without the TRAC-Hinge Disulfide (variants v33048 and v33047, respectively). These two variants acted as controls as described in more detail below.

All TCR fusion proteins comprised the TRAC/84.2_TRBC/79 disulfide. The combinations of mutations tested are shown on Table 12.1.

Each combination of mutations was introduced into an anti-gp100 TCR fused to an Fc. The TCR fusion proteins were constructed in a one-armed format with the beta chain fused to the Fc as described in Example 1. All sequences were preceded by the signal peptide: MRPTWAWWLFLVLLLALWAPARG [SEQ ID NO:22]. Vector inserts were prepared and cloned into the pTT5 vector for expression as described in Example 1.

The TCR fusion proteins were initially tested for expression in 0.8 mL of HEK293-6E cells as described in Example 4. Expression was confirmed by SDS-PAGE and variants with observable protein expression bands were subsequently expressed in 250 mL of HEK293-6E cells as described in Example 4 but with a DNA ratio of 40:40:20 for alpha chain, beta chain-Fc(A), and Fc (B). After the initial 18-22 hour incubation, the cells were incubated at 32° C. for 5 days and subsequently purified and characterized by UPLC-SEC as described in Example 4 and DSC as described in Example 6. Binding of the TCR fusion proteins to the target peptide-MHC complex was confirmed using flow cytometry as described in Example 10.

The results are shown in Table 12.1. As can be seen from Table 12.1, all tested combinations were successfully expressed as TCR fusion proteins. TCR fusion proteins comprising the tested combinations were compared against variant v33048 which contains the TRAC-Hinge Disulfide. The expression yield for the TCR fusion proteins comprising alternative disulfide bonds was generally lower than that for variant v33048. The amount of HMW species observed for TCR fusion proteins comprising alternative disulfide bonds was comparable to that for variant v33048, with the amounts observed for variants v33049 and v33055 being the most similar. All other variants comprised under 15% HMW species.

All TCR fusion proteins comprising an alternative C-terminal disulfide bond (i.e. variants v33048, v33049, v33050, v33051, v33052, v33053, v33054, v33056 and v33057) showed an increase in Tm compared to variant v33047 which lacked an alternative C-terminal disulfide bond. Additionally, several of the variants comprising alternative C-terminal disulfide bonds demonstrated an increase in Tm compared to variant v33048 which comprises the TRAC-Hinge Disulfide (see variants v33050, v33053, v33054, v33056 and v33057 in Table 12.1).

In summary, all the alternative C-terminal disulfide bonds tested were successful in improving the stability of the TCR fusion protein and can be used as an alternative to the TRAC-Hinge Disulfide.

TABLE 12.1
Characteristics of TCR Fusion Proteins Comprising Alternative C-Terminal Disulfide Bonds

| | Interchain Disulfides | | | | | | | | |
|---|---|---|---|---|---|---|---|---|---|
| | | Disulfide | | Disulfide | Disulfide | Disulfide | Disulfide | Disulfide | Disulfide |
| Variant | IC | TRAC 84.2 - | Hinge | TRAC 122 - | TRAC 124 - | TRAC 125 - | TRAC 126 - | TRAC 127 - | TRAC 128 |
| Number | Disulfide | TRBC 79 | Disulfide | TRCB 12 | TRCB 11 | TRCB 11 | TRCB 11 | TRCB 11 | TRCB 11 |
| v21230 | x | | | | | | | | |
| v33048 | | x | x | | | | | | |
| v33047 | | x | | | | | | | |
| v33049 | | x | | | | | | x | |
| v33050 | | x | | | | | | x | |
| v33051 | | x | | | x | | | | |
| v33052 | | x | | | | x | | | |
| v33053 | | x | | | | | x | | |
| v33054 | | x | | | | | | | x |
| v33055 | | x | | x | | | | | |
| v33056 | | x | | x | | | | | |
| v33057 | | x | | x | | | | | |

| | Intrachain | | | | | | |
|---|---|---|---|---|---|---|---|
| | Disulfide | Point Mutations | | | Measured | | |
| | Disulfide | TRBC | TRBC | TRAC | TCR | HMW | pA |
| Variant | TRAC 39 | 6.VAL −> | 36.HIS −> | 26.THR−> | Tm | Species | Yield |
| Number | TRAC 85 | ILE | PHE | ILE | (° C.) | (%) | (mg/L) |
| v21230 | | | | | 53.5 | 3.8 | 33.6 |
| v33048 | x | x | x | | 58.7 | 6.6 | 32.4 |
| v33047 | x | x | x | | 54.8 | 11.2 | 23.0 |
| v33049 | | | | | 55.5 | 6.5 | 20.0 |
| v33050 | x | x | x | | 60.3 | 11.8 | 17.2 |
| v33051 | x | x | x | | 57.2 | 9.5 | 17.6 |
| v33052 | x | x | x | | 58.5 | 11.3 | 16.8 |
| v33053 | x | x | x | | 60.2 | 10.1 | 16.8 |
| v33054 | x | x | x | | 60.0 | 10.2 | 16.4 |
| v33055 | | | | | 55.7 | 6.2 | 14.4 |
| v33056 | x | x | x | | 60.4 | 11.5 | 11.5 |
| v33057 | x | x | x | x | 59.6 | 14.3 | 14.3 |

### Example 13: Bispecific TCR Fusion Proteins Comprising Stabilizing Mutations

This Example describes the construction, expression and characterization of CD3-engaging bispecific TCR fusion proteins comprising an anti-CD3 scFv or Fab and one or more TCR.

The TCR fusion proteins were expressed as heterodimers with one or two TCR components and one anti-CD3 component. The anti-CD3 component was either a humanized OKT3 or UCHT-1 as paratope in an scFv or a canonical Fab format (see International Patent Publication Nos. WO 2017/008169 and WO 2010/133828).

The TCR component was either an anti-gp100 TCR or an anti-NY-ESO1 1G4-HA with substitutions in the CDRs to produce different affinities as shown in Table 13.1 (see Li et al., 2005, Nature Biotechnology, 23(3):349-354 and International Patent Publication No. WO 2011/001152).

All TCR components comprised the following stabilizing mutations in the TCR constant domain: TRAC/4.VAL->ILE, TRAC/85.ALA->VAL, TRAC/105.ALA->SER TRBC/6.VAL->LEU, TRBC/36.HIS->PHE, TRBC/86.SER->THR, TRBC/45.3->THR, Δ_TRBC/84.4-85.4->GLY-ASN, TRAC/84.2->CYS_TRBC/79.SER->CYS (disulfide) and TRAC-Hinge Disulfide.

The TCR fusion proteins comprised a human IgG1 heterodimeric Fc comprising CH3 domain amino acid substitutions promoting the formation of a heterodimeric Fc as described in Example 1. All bispecific variants included the following CH2 domain amino acid substitutions which knock out FcTR binding: L234A, L235A and D265S.

The following TCR fusion protein formats were employed: One-armed beta fusion (“OA”; see FIG. 1H), Bispecific beta-fusion (“Hybrid”; see FIG. 1L), 2×1 Bispecific beta-fusion (“2×1”; see FIG. 10) and Bispecific tandem beta-fusion (“1×1 Tandem”; see FIG. 1N).

TABLE 13.1
CDR Sequences and Affinities of Modified Anti-gp100 and Anti-NY-ESO1 TCRs

| | | | | | | | | | Reported |
|---|---|---|---|---|---|---|---|---|---|
| | CDR Sequence | | | | | | | | Affinity |
| | | SEQ | | SEQ | | SEQ | | SEQ | for Target |
| TCR | CDR2α | ID NO | CDR3α | ID NO | CDR2B | ID NO | CDR3β | ID NO | (pM) |
| gp100-A | IRSNERE | 25 | ATDGSTPMQ | 26 | SWAQGD | 27 | ASSWGAPY | 28 | 0.04 |
| gp100-B | IRSNERE | 25 | ATDGDTPLV | 29 | SWAQGD | 27 | ASSHGAPY | 30 | 0.065 |
| gp100-C | IRSNERE | 25 | ATDGDTPLV | 29 | SWGTGD | 31 | ASSHGAPY | 30 | 0.31 |
| gp100-D | IRSNERE | 25 | ATDGDTPLV | 29 | SWAVGN | 32 | ASSIGGPY | 33 | 1.2 |
| NY-ESO1-A | ITPWQRE | 34 | AVRPLLDGTYIPT | 35 | SVAIQT | 36 | ASSYLGNTGELF | 37 | 0.026 |
| NY-ESO1-B | IQSSQRE | 38 | AVRHTSNGYFPPT | 39 | SVGAGT | 40 | ASSYLGNTGELF | 37 | 0.98 |
| NY-ESO1-C | ISPWQRE | 41 | AVRPLLDGTYIPT | 35 | SVAIQT | 36 | ASSYVGDTGELF | 42 | 8.4 |

Vector inserts encoding each bispecific TCR fusion protein were prepared and cloned into the pTT5 vector for expression as described in Example 1. All sequences were preceded by the signal peptide: MRWTWAWWLFLVLLLALWAPARG [SEQ ID NO:22].

The TCR fusion proteins were initially tested for expression in 0.8 mL of HEK293-6E cells as described in Example 4. Expression was confirmed by SDS-PAGE and variants with observable protein expression bands were subsequently expressed in 250 mL of HEK293-6E cells as described in Example 4. After the initial 18-22 hour incubation, the cells were incubated at 32° C. and purified as described in Example 4. Amounts of 11HMW species were measured by UPLC-SEC as described in Example 4. Binding of the anti-gp 100 TCR fusion proteins to the target peptide-MHC complex was measured by flow cytometry as described in Example 10.

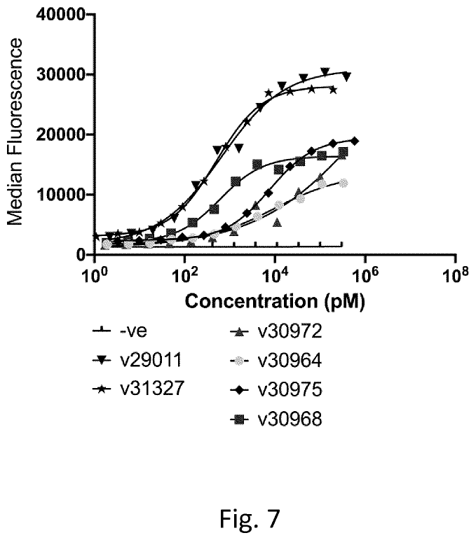

The results are shown in Table 13.2 and FIG. 7.

TABLE 13.2
Characteristics of Bispecific TCR Fusion Proteins

| | | | | | | Anti- |
|---|---|---|---|---|---|---|
| | Fusion | | Antibody | Protein A | HMW | gp100 |
| | Protein | TCR | Component | Yield | Species | EC₅₀ |
| Variant # | Format | Component | (Format) | (mg/L) | (%)¹ | (pM) |
| v29011 | OA | gp100-A | — | 28.8 | 14.4 | 463 |
| v30973 | OA | gp100-B | — | 36.8 | 14.9 | 704 |
| v30974 | OA | gp100-C | — | 35.2 | 13.9 | 685 |
| v30975 | OA | gp100-D | — | 46.4 | 14.2 | 7790 |
| v31327 | Hybrid | gp100-A | OKT3 (scFv) | 24 | 21.6 | 503 |
| v31328 | Hybrid | gp100-A | UCHT1 (scFv) | 22.4 | 23.9 | 790 |
| v30962 | Hybrid | gp100-B | OKT3 (scFv) | 24.8 | 16.3 | 633 |
| v30963 | Hybrid | gp100-C | OKT3 (scFv) | 32 | 16 | 479 |
| v30964 | Hybrid | gp100-D | OKT3 (scFv) | 39.2 | 16.1 | 9330 |
| v31307 | 2 × 1 | gp100-A | OKT3 (Fab) | 28.8 | 9.8 | 384 |
| v30966 | 2 × 1 | gp100-B | OKT3 (Fab) | 28 | 7.8 | 338 |
| v30967 | 2 × 1 | gp100-C | OKT3 (Fab) | 42.4 | 6.7 | 330 |
| v30968 | 2 × 1 | gp100-D | OKT3 (Fab) | 47.2 | 7.6 | 605 |
| v31308 | 1 × 1 | gp100-A | OKT3 (Fab) | 32 | 9.5 | 1271 |
| | tandem | | | | | |
| v30970 | 1 × 1 | gp100-B | OKT3 (Fab) | 37.6 | 7.0 | 796 |
| | tandem | | | | | |
| v30971 | 1 × 1 | gp100-C | OKT3 (Fab) | 42.4 | 8.7 | 1100 |
| | tandem | | | | | |
| v30972 | 1 × 1 | gp100-D | OKT3 (Fab) | 41.6 | 8.6 | >10000 |
| | tandem | | | | | |
| v30976 | OA | — | OKT3 (scFv) | 45.6 | 8.3 | No |
| | | | | | | binding |
| v31181 | 2 × 1 | NY-ESO1-A | OKT3 (Fab) | 40 | 5.2 | — |
| v31182 | 2 × 1 | NY-ESO1-B | OKT3 (Fab) | 54.4 | 3.7 | — |
| v31183 | 2 × 1 | NY-ESO1-C | OKT3 (Fab) | 33.6 | 5.0 | — |

¹As determined by UPLC-SEC

As can be seen from Table 13.2, all tested formats expressed successfully, with protein A purified yields ranging from 24 mg/L to 54 mg/L. The amount of high molecular weight species present ranged from 3.700 to 23.90%. Formats comprising a Fab or a second TCR maintained similar expression yields and HMW species.

All variants comprising an anti-gp100 TCR maintained binding to the target MHC-peptide complex. The GA variant v29011 and the bispecific variant v31327 had almost identical binding curves (see FIG. 7), suggesting that adding a second binding moiety (such as an anti-CD3 component) to the TCR fusion protein does not affect the binding of the TCR component to its target MHC-peptide complex. FIG. 7 also shows that an expected reduced response in binding was observed with the variants comprising the lower affinity anti-gp 100 TCR component: v30972, v30964, v30975 and v30968, when compared to the higher affinity anti-gp 100 TCR controls: v29011 and v31327.

Increasing the number of TCR moieties on the fusion protein was observed to improve the binding response. As can be seen in Table 13.2, the 2×1 variants (v31307, v30966, v30967 and v30968) all showed an improvement in affinity over the corresponding variants comprising a single TCR component. The 2×1 variant v30968 comprising two copies of the weakest affinity anti-gp100 TCR component showed a decrease in EC₅₀ compared to variants v30975, 30964, and 30972, which comprise a single copy of this TCR component (see FIG. 7).

### Example 14: Effect of Different TCR Germline Sequences on Expression of TCR Fusion Proteins

To determine if germline sequence affects expression and stability of TCR fusion proteins, the CDRs from an anti-gp100 TCR were grafted onto different TCR germline framework sequences as described below.

In order to determine the effect of different germline sequences on TCR stability only the framework region in the variable domains of the TCR component of the TCR fusion proteins was varied. The CDR boundaries were selected based on IMGT definitions and the CDR sequences from the alpha and beta chains of the anti-gp100 TCR were used to replace the corresponding CDR regions in alternative germline sequences. The germline sequences of the top five alpha and top five beta sequences were identified based on frequency determined using the IMGT/GeneFrequency tool accessed through the International ImMunoGeneTics Information System (IMGT®) website. The top five alpha sequences were identified as TRAV19, TRAV14/DV4, TRAV17, TRAV9-2 and TRAV8-4, and the top five beta sequences were identified as TRBV28, TRBV30, TRBV19, TRBV27 and TRBV5-1. The natural germline sequences in the anti-gp100 TCR are TRAV17 and TRBV19.

The alpha and beta sequences comprising the grafted CDR sequences were then combined so that each alpha sequence was matched with each beta sequence for a total of 25 combinations. TCR fusion proteins comprising each combination were constructed in a one-armed format with the beta chain fused to the Fc as described in Example 1. All TCR fusion proteins contained the IC Disulfide and the TRAC-Hinge Disulfide. All sequences were preceded by the signal peptide: MRPTWAWWLFLVLLLALWAPARG [SEQ ID NO:22]. Vector inserts were prepared and cloned into the pTT5 vector for expression as described in Example 1.

The TCR fusion proteins were initially tested for expression in 0.8 mL of HEK293-6E cells as described in Example 4. Expression was confirmed by SDS-PAGE and variants with observable protein expression bands were subsequently expressed in 250 mL of HEK293-6E cells as described in Example 4 but with a DNA ratio of 40:40:20 for alpha chain, beta chain-Fc(A), and Fc (B). After the initial 18-22 hour incubation, the cells were incubated at 32° C. and subsequently purified and characterized by UPLC-SEC as described in Example 4 and DSC as described in Example 6.

The results are shown in Table 14.1.

TABLE 14.1
Expression of TCR Fusion Proteins Comprising Different
Germline Sequences

| | | | Protein A | HMW | |
|---|---|---|---|---|---|
| Variant | Alpha | Beta | Yield | Species | TCR |
| # | Germline | Germline | (mg/L) | (%)¹ | Tm (° C.) |
| v31114 | TRAV19 | TRBV28 | 8.9 | 48.9 | N/D² |
| v31115 | TRAV19 | TRBV30 | No Ex³ | — | — |
| v31116 | TRAV19 | TRBV19 | No Ex | — | — |
| v31117 | TRAV19 | TRBV27 | No Ex | — | — |
| v31118 | TRAV19 | TRBV5-1 | No Ex | — | — |
| v31124 | TRAV14/DV4 | TRBV28 | 14 | 56.3 | N/D |
| v31125 | TRAV14/DV4 | TRBV30 | No Ex | — | — |
| v31126 | TRAV14/DV4 | TRBV19 | No Ex | — | — |
| v31127 | TRAV14/DV4 | TRBV27 | 9.0 | 71.8 | N/D |
| v31128 | TRAV14/DV4 | TRBV5-1 | 7.6 | 82.2 | N/D |
| v31129 | TRAV17 | TRBV28 | 24.2 | 14.6 | 53.5 |
| v31130 | TRAV17 | TRBV30 | No Ex | — | — |
| v31131 | TRAV17 | TRBV19 | 24.1 | 10.4 | 53.3 |
| v31132 | TRAV17 | TRBV27 | 10.9 | 71.4 | N/D |
| v31133 | TRAV17 | TRBV5-1 | 13.5 | 78.4 | N/D |
| v31134 | TRAV8-4 | TRBV28 | 7.5 | 42.9 | N/D |
| v31135 | TRAV8-4 | TRBV30 | No Ex | — | — |
| v31136 | TRAV8-4 | TRBV19 | No Ex | — | — |
| v31137 | TRAV8-4 | TRBV27 | No Ex | — | — |
| v31138 | TRAV8-4 | TRBV5-1 | No Ex | — | — |
| v31144 | TRAV9-2 | TRBV28 | 7.9 | 55.3 | N/D |
| v31145 | TRAV9-2 | TRBV30 | No Ex | — | — |
| v31146 | TRAV9-2 | TRBV19 | No Ex | — | — |
| v31147 | TRAV9-2 | TRBV27 | No Ex | — | — |
| v31148 | TRAV9-2 | TRBV5-1 | No Ex | — | — |

¹As determined by UPLC-SEC
²N/D = not determined due to high levels of HMW species
³No Ex = no observable expression by SDS-PAGE

As can be seen from Table 14.1, only two variants produced soluble protein which was monodispersed: v31129 and v31131. Variant v31131 comprises the germline sequences TRAV17 and TRBV19, which are natural to the anti-gp100 TCR and, as such, was expected to express. Variant v31129 comprises the germline sequences TRAV17 and TRBV28 and therefore includes the same TRAV sequence as the natural anti-gp100 TCR. The amount of HMW species observed for variant v31129 were slightly higher than for v31131, but the thermal stability was similar: 53.5° C. vs 53.3° C. TRBV28 therefore appears to be compatible with this set of TCR CDRs and TRAV17. Overall, the results in Table 14.1 suggest that the set of CDRs from this anti-gp100 TCR are not compatible with most other germlines and that the natural germline sequences likely provide optimal stability for a given TCR sequence.

### Example 15: LONG-TERM STABILITY OF STABILIZED TCR FUSION PROTEINS

In order to assess the long-term stability of TCR fusion protein, select variants described in Examples 4, 6, 9, 11 and 14 were incubated at 40° C. and the amount of HMW species was assessed by UPLC-SEC as described below.

The TCR fusion proteins tested are listed in Table 15.1. The protein concentration of these selected stabilized TCR fusion proteins was adjusted to 1 mg/mL in PBS buffer. 200 ul of each variant solution were sealed in an Eppendorf tube and incubated in an incubator set at 40° C. for 14 or 30 days. A 40 ul sample of each variant was taken at the following timepoints: 0, 3, 7, 10 and 14 days (for 14-day incubations) or 0, 5, 20 and 30 days (for 30-day incubations). Samples were immediately frozen at −80° C. At the completion of the incubation period all samples were thawed and the amount of HMW species was assessed by UPLC-SEC as described in Example 4.

The results are shown in Table 15.1.

TABLE 15.1
Change in Amount of HMW Species for TCR Fusion Proteins Incubated at 40° C.

| | | Change in HMW | Change in Desired |
|---|---|---|---|
| Variant | | Species | Species |
| # | Stabilizing Mutations | (% increase/day) | (% loss/day) |
| v21230 | IC Disulfide | 0.89 | 0.92 |
| v22712 | TRBC/6.VAL->LEU | 0.56 | 0.55 |
| | IC Disulfide | | |
| v22752 | IC Disulfide, TRAC-Hinge Disulfide | 0.08 | 0.08 |
| v28881 | TRBC/6.VAL->ILE | 0.12 | 0.12 |
| | IC Disulfide | | |
| v28897 | TRAC/85.ALA->VAL | 0.01 | 0.01 |
| | IC Disulfide, TRAC-Hinge Disulfide | | |
| v28902¹ | TRAC/84.2.LEU->CYS-TRBC/79.SER- | 0.79 | 0.87 |
| | >CYS (Disulfide), TRAC-Hinge Disulfide | | |
| v28907 | TRAC/85.ALA->VAL, TRBC/45.3.VAL- | 0.30 | 0.35 |
| | >THR, TRAC/105.ALA->SER | | |
| | IC Disulfide | | |
| v29011 | TRAC/4.VAL->ILE, TRAC/85.ALA->VAL, | 0.46 | 0.48 |
| | TRBC/6.VAL->LEU, TRBC/36.HIS->PHE, | | |
| | TRBC/86.SER->THR, TRBC/45.3.VAL- | | |
| | >THR, TRAC/105.ALA->SER, | | |
| | ATRBC/84.4-85.4 | | |
| | TRAC-Hinge Disulfide, IC Disulfide | | |
| v31085 | TRAC/84.2.LEU->CYS-TRBC/79.SER- | 0.15 | 0.19 |
| | >CYS (Disulfide), TRAC-Hinge Disulfide, | | |
| | TRAC/39.VAL->CYS-TRAC/85.ALA- | | |
| | >CYS (Disulfide) | | |
| v31097 | TRAC/85.ALA->VAL, TRBC/6.VAL->ILE, | 0.0 | 0.0 |
| | TRBC/36.HIS->PHE, | | |
| | IC Disulfide, TRAC-Hinge Disulfide | | |
| v31099 | TRAC/39.VAL->ILE, TRAC/85.ALA- | 0.03 | 0.03 |
| | >VAL, TRBC/36.HIS->PHE | | |
| | IC Disulfide, TRAC-Hinge Disulfide | | |
| v31129² | IC Disulfide, TRAC-Hinge Disulfide | 0.70 | 0.19 |
| v31131 | TRAC/84.2.LEU->CYS-TRBC/79.SER- | 0.18 | 0.18 |
| | >CYS (Disulfide), TRAC-Hinge Disulfide | | |

¹This variant includes the mutations TRAC/1.5.GLN->LYS and TRBC/97.GLN->ASP as described in Example 1
²This variant comprises the TRAV17 and TRBV28 germline sequences

The % change in each species was determined by the difference between the first measurement (Day 0) and the final measurement (Day 14 or 30) and averaged over the length of incubation. For example, a reported increase in HMW species of 0.70% is equivalent to an increase of 9.8% after a 14-day incubation.

As can be seen from Table 15.1, the TCR fusion protein containing only the IC Disulfide (v21230) showed the greatest rate of increase of HMW species (aggregation) of all variants tested. All TCR fusion proteins comprising combinations of stabilizing mutations showed reduced amounts of HM/1W species compared to variant v21230, with variants v28897, v31097 and v31099 showing a more than 10-fold decrease in the rate of formation of HMW species compared to v21230.

The results shown in Table 15.1 indicate that including combinations of the stabilization mutations described in the preceding Examples in a TCR fusion protein increases the long-term colloidal stability of the fusion protein as compared to a TCR fusion protein comprising the IC Disulfide alone.

### Example 16: Production and Characterization of Multivalent TCR Fusion Proteins

Increasing the number of TCR moieties on a TCR fusion protein could improve binding to the target peptide-MHC complex (as shown in Example 13) or provide additional biological function by allowing binding to multiple peptide-MHC complexes simultaneously. Multivalent TCR fusion proteins with a valency of up to four TCRs were produced and characterized as described below.

TCR fusion proteins were constructed in the following formats:
(1) Bispecific beta-fusion (“1×1”; see FIG. 1L)
(2) 2×1 Bispecific beta-fusion (“2×1”; see FIG. 1O)
(3) 3×1 Bispecific beta-fusion (“3×1”; see FIG. 1P)
(4) 4×1 Bispecific C-terminal beta-fusion (“4×1 tandem”; see FIG. 1Q)
(5) 4×1 Bispecific light chain beta-fusion (“4×1 LC”; see FIG. 1R).

The antibody component in each case was an anti-CD3 Fab or scFv. The TCR components were wildtype 1G4-WT anti-NY-ESO1 TCR (“NY-ESO1-WT”), affinity matured 1G4-33A anti-NY-ESO1 TCR (“NY-ESO1-33A”) (Li, et al., 2005, Nat. Biotechnol., 23:349-354) or anti-PRAME TCR as outlined in Table 16.1. All TCR sequences contained the TRAC/84.2_TRBC/79 disulfide, TRAC/39.VAL->CYS_TRAC/85.ALA->CYS (disulfide) and the TRAC-Hinge Disulfide stabilizing mutations.

Vector inserts encoding each TCR fusion protein were prepared and cloned into the pTT5 vector for expression as described in Example 1. All sequences were preceded by the signal peptide

[SEQ ID NO: 22]
MRPTWAWWLFLVLLLALWAPARG.

The TCR fusion proteins were initially tested for expression in 0.8 mL of HEK293-6E cells as described in Example 4. Expression was confirmed by SDS-PAGE and variants with observable protein expression bands were subsequently expressed in 250 mL of HEK293-6E cells as described in Example 4. After the initial 18-22 hour incubation, the cells were incubated at 32° C. and purified as described in Example 4. Monodispersity was measured by UPLC-SEC as described in Example 4.

The results are shown in Table 16.1.

TABLE 16.1
Expression and Monodispersity of Multimeric TCR Fusion Proteins

| | | | Protein A | |
|---|---|---|---|---|
| | | | Expression | Monodispersity |
| Variant # | TCR Target | Format | (mg/L) | post-SEC (%) |
| v32545¹ | NY-ESO1 | 1 × 1 | 16.2 | 100 |
| v32546¹ | NY-ESO1 | 2 × 1 | 18.0 | 96.6 |
| v32547¹ | NY-ESO1 | 3 × 1 | 16.1 | 98.0 |
| v32548¹ | NY-ESO1 | 4 × 1 tandem | 6.6 | 100 |
| v32549¹ | NY-ESO1 | 4 × 1 LC | 5.9 | 100 |
| v32550² | NY-ESO1 | 1 × 1 | 16.6 | 97.2 |
| v32551² | NY-ESO1 | 2 × 1 | 17.2 | 98.4 |
| v32552² | NY-ESO1 | 3 × 1 | 18.4 | 96.7 |
| v32553² | NY-ESO1 | 4 × 1 tandem | 8.7 | 96.0 |
| v32554² | NY-ESO1 | 4 × 1 LC | 8.9 | 95.6 |
| v32569 | PRAME | 1 × 1 | 5.4 | 97.8 |
| v32570 | PRAME | 2 × 1 | —³ | — |
| v32571 | PRAME | 3 × 1 | —³ | — |
| v32572 | PRAME | 4 × 1 tandem | —³ | — |
| v32573 | PRAME | 4 × 1 LC | —³ | — |

¹Wildtype 1G4-WT anti-NY-ESO1 TCR
²Affinity matured 1G4-33A anti-NY-ESO1 TCR
³No observable expression

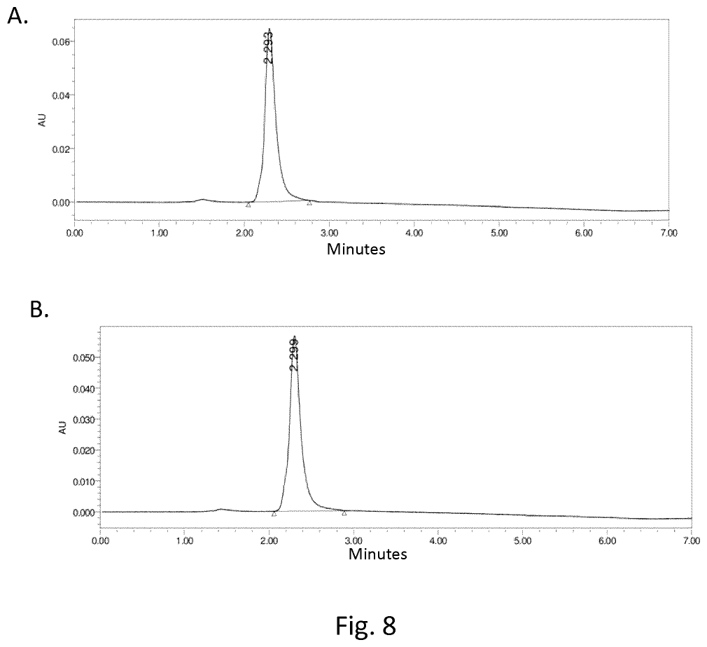

As can be seen from Table 16.1, TCR fusion proteins comprising the WT anti-NY-ESO1 TCR, the high affinity anti-NY-ESO1 TCR or the anti-PRAME TCR in a lxi format expressed successfully. TCR fusion proteins comprising up to four anti-NY-ESO1 TCRs were expressed in sufficient quantities to be analyzed and subsequently purified as monodispersed samples. Exemplary UPLC-SEC traces for two 4×1 TCR fusion proteins, v32548 and v32549, are shown in FIG. 8. The fusion proteins comprising anti-PRAME TCRs appeared to be generally less stable than the fusion proteins comprising anti-NY-ESO1 TCRs. While the stabilizing mutations sufficiently stabilized the anti-PRAME TCR to allow successful expression of the lxi format, expression levels were lower than those for either of the anti-NY ES01 TCRs in the same lxi format, indicating that the anti-PRAME TCR is likely an inherently low-expressing TCR.

### Example 17: T2 T-Cell Dependent Cytotoxicity Assay of Stabilized TCR Fusion Proteins

TCR fusion proteins from Example 16 that are specific for the NY-ESO1 peptide were tested for cell killing against cells having their target peptide-MHC complex in a T2 T-cell dependent cytotoxicity assay (TDCC). The TCR fusion proteins tested are listed in Table 17.1.

T2 cells (ATCC CRL-1992) were cultured in RPMI1640+10% FBS in T75 flasks at 37° C.+5% CO₂ for at least two passages before use. Cells were pulsed by resuspension in culture media with 10 μM NY-ESO-1 peptide (SLLMWITQC [SEQ ID NO:23]) and 100 ng/mL β-2-microglobulin (Sino Biological, Beijing, China) and incubated for 24 hours at 37° C.+5% CO₂. A TDCC assay was prepared in RPMI-1640+10% FBS+1% Penicillin/Streptomycin assay media in 96-well U-bottom plates. TCR fusion proteins were prepared by serial dilutions of 1:5 with concentration ranges of 1 μM-0.05 μM, 60 μL/well. The T2 cells were stained with 2 μM carboxyfluorescein succinimidyl ester (CFSE) using the manufacturer's protocol (ThermoFisher, Waltham, MA). Post-staining, T2 cells were resuspended in RPMI1640+10% FBS and mixed with freshly thawed T-cells (BioIVT, Westbury, NY) at 5:1 ratio. The cell mixture was added at 60 μL/well and the plates were incubated at 37° C.+5% CO₂ for 48 hours. Post-incubation, samples were transferred to 96-well V-bottom plates and washed 2× with FACS buffer (PBS+2% FBS). Samples were resuspended in 2 μg/mL of 7-aminoactinomycin D (7-AAD) (BioLegend, San Diego, CA) at 50 μL/well. The plate was incubated at room temperature for 15 minutes, followed by two washes with FACS buffer. Samples were resuspended in 50 μL/well FACS buffer and the plates were analyzed by flow cytometry using a BD Fortessa™ X-20 (BD Biosciences, San Jose, CA).

The measured EC₅₀ values are summarized in Table 17.1. See also FIG. 10. The TCR fusion proteins contain either the NY-ESO1-WT paratope (affinity: 32000 nM) or the NY-ESO1-33A paratope (affinity: 254 nM) as noted in Table 17.1.

TABLE 17.1
EC₅₀ of Multimeric TCR Fusion Proteins in T2 T Cell Dependent
Cytotoxicity Assay

| | | | NY-ESO1 |
|---|---|---|---|
| Variant # | Format | EC₅₀ (nM) | Paratope |
| v31185 | One-Armed beta- | No Activity | NY-ESO-WT |
| | fusion - DS | | |
| v32545 | Bispecific beta-fusion | Minimal Activity¹ | NY-ESO1-WT |
| v32546 | 2 × 1 Bispecific beta- | Minimal Activity¹ | NY-ESO1-WT |
| | fusion | | |
| v32547 | 3 × 1 Bispecific beta- | Minimal Activity¹ | NY-ESO1-WT |
| | fusion | | |
| v32548 | 4 × 1 Bispecific | Minimal Activity¹ | NY-ESO1-WT |
| | C-terminal beta-fusion | | |
| v32549 | 4 × 1 Bispecific light | Minimal Activity¹ | NY-ESO1-WT |
| | chain beta-fusion | | |
| v32550 | Bispecific beta-fusion | 28.3 | NY-ESO1-33A |
| v32551 | 2 × 1 Bispecific beta- | 8.4 | NY-ESO1-33A |
| | fusion | | |
| v32552 | 3 × 1 Bispecific beta- | 2.0 | NY-ESO1-33A |
| | fusion | | |
| v32553 | 4 × 1 Bispecific C- | 2.8 | NY-ESO1-33A |
| | terminal beta-fusion | | |
| v32554 | 4 × 1 Bispecific light | Minimal Activity¹ | NY-ESO1-33A |
| | chain beta-fusion | | |

¹Activity observed only at μM concentrations

As can be seen from Table 17.1 and FIG. 10, TCR-anti-CD3 bispecific proteins were able to selectively kill target cells displaying the desired MHC-peptide complex. Targeted cell killing occurred only with the bispecific molecule—the one-armed TCR molecule that lacked the anti-CD3 paratope (v31185) produced no killing effect at any observed concentration. This demonstrates that the multivalent format is required for cell killing. Additionally, there was about a 10-fold increase in cell killing from the 1× variant to the 3× variant suggesting an avidity affect with the 3× variant providing the greatest enhancement in cell killing. No further increase in EC₅₀ was observed with the 4× variant, which may be due to a maximum potency having already been achieved or the 4× format may be a non-optimal format for the NY-ESO1 TCR. The variants comprising the lower affinity TCR (NY-ESO1-WT) all showed a minimal response regardless of the valency of the TCRs.

Overall, these results demonstrate the TCR-anti-CD3 bispecific proteins can selectively kill target cells and this activity can be increased through increasing the valency of the TCR components.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Modifications of the specific embodiments described herein that would be apparent to those skilled in the art are intended to be included within the scope of the following claims.

SEQUENCE TABLE

| SEQ ID | | |
|---|---|---|
| NO | Description | Sequence |
| 1 | Human TCR alpha chain constant | NIQNPDPAVYQLRDSKSSDKSVCLFTDFDSQTN |
| | region (TRAC) | VSQSKDSDVYITDKTVLDMRSMDFKSNSAVA |
| | | WSNKSDFACANAFNNSIIPEDTFFPSPESS |
| 2 | Human TCR beta chain constant | EDLNKVFPPEVAVFEPSEAEISHTQKATLVCLA |
| | region (TRBC1) | TGFFPDHVELSWWVNGKEVHSGVSTDPQPLKE |
| | | QPALNDSRYCLSSRLRVSATFWQNPRNHFRCQ |
| | | VQFYGLSENDEWTQDRAKPVTQIVSAEAWGR |
| | | AD |
| 3 | Human TCR beta chain constant | EDLKNVFPPEVAVFEPSEAEISHTQKATLVCLA |
| | region (TRBC2) | TGFYPDHVELSWWVNGKEVHSGVSTDPQPLK |
| | | EQPALNDSRYCLSSRLRVSATFWQNPRNHFRC |
| | | QVQFYGLSENDEWTQDRAKPVTQIVSAEAWG |
| | | RAD |
| 4 | Human TCR alpha chain constant | NIQNPDPAVYQLRDSKSSDKSVCLFTDFDSQTN |
| | region (TRAC) including the | VSQSKDSDVYITDKTVLDMRSMDFKSNSAVA |
| | cysteine residue at position | WSNKSDFACANAFNNSIIPEDTFFPSPESSC |
| | TRAC/128 | |
| 5 | TCRα hinge sequence | ESSCDVKLVEKSFET |
| 6 | TCRß hinge sequence | DCGFTS |
| 7 | TCRγ hinge sequence | DVITMDPKDNCSKDAN |
| 8 | TCRδ hinge sequence | DHVKPKETENTKQPSKSCHKPK |
| 9 | IgG1 hinge sequence | EPKSCDKTHTCPPCP |
| 10 | IgG2 hinge sequence | ERKCCVECPPCP |
| 11 | IgG3-H1 hinge sequence | ELKTPLGDTTHTCPRCP |
| 12 | IgG3-H2, -H3 & -H4 hinge sequence | EPKSCDTPPPCPRCP |
| 13 | IgG4 hinge sequence | ESKYGPPCPSCP |
| 14 | IgA2 hinge sequence | VPPPPP |
| 15 | IgG1 full hinge sequence | EPKSCDKTHTCPPCPAPELLGG |
| 16 | IgG1 upper hinge sequence | EPKSCDKTHT |
| 17 | IgG1 core hinge sequence | CPPCP |
| 18 | IgG1 lower hinge sequence | APELLGG |
| 19 | IgG1 partial hinge sequence | EPKSC |
| 20 | Human IgG1 Fc sequence 231-447 | APELLGGPSVFLFPPKPKDTLMISRTPEVTCVV |
| | (EU-numbering) | VDVSHEDPEVKFNWYVDGVEVHNAKTKPREE |
| | | QYNSTYRVVSVLTVLHQDWLNGKEYKCKVSN |
| | | KALPAPIEKTISKAKGQPREPQVYTLPPSRDELT |
| | | KNQVSLTCLVKGFYPSDIAVEWESNGQPENNY |
| | | KTTPPVLDSDGSFFLYSKLTVDKSRWQQGNVF |
| | | SCSVMHEALHNHYTQKSLSLSPGK |
| 21 | IgG1 partial hinge sequence | EPKSCDKTHTCPPCP |
| 22 | Signal sequence | MRPTWAWWLFLVLLLALWAPARG |
| 23 | NY-ESO1 peptide | SLLMWITQC |
| 24 | gp100 peptide | YLEPGPVTA |
| 25 | anti-gp100 CDR2a | IRSNERE |
| 26 | anti-gp100 CDR3a | ATDGSTPMQ |
| 27 | anti-gp100 CDR2b | SWAQGD |
| 28 | anti-gp100 CDR3b | ASSWGAPY |
| 29 | anti-gp100 CDR3a | ATDGDTPLV |
| 30 | anti-gp100 CDR3b | ASSHGAPY |
| 31 | anti-gp100 CDR2b | SWGTGD |
| 32 | anti-gp100 CDR2b | SWAVGN |
| 33 | anti-gp100 CDR3b | ASSIGGPY |
| 34 | anti-NY-ESO1 CDR2a | ITPWQRE |
| 35 | anti-NY-ESO1 CDR3a | AVRPLLDGTYIPT |
| 36 | anti-NY-ESO1 CDR2b | SVAIQT |
| 37 | anti-NY-ESO1 CDR3b | ASSYLGNTGELF |
| 38 | anti-NY-ESO1 CDR2a | IQSSQRE |
| 39 | anti-NY-ESO1 CDR3a | AVRHTSNGYFPPT |
| 40 | anti-NY-ESO1 CDR2b | SVGAGT |
| 41 | anti-NY-ESO1 CDR2a | ISPWQRE |
| 42 | anti-NY-ESO1 CDR3b | ASSYVGDTGELF |
| 43 | Human TCR beta chain constant | EDLNKVFPPEVAVFEPSEAEISHTQKATLVCLA |
| | region (TRBC1) including Cys → | TGFFPDHVELSWWVNGKEVHSGVSTDPQPLKE |
| | Ala mutation at position 85.1 | QPALNDSRYALSSRLRVSATFWQNPRNHFRCQ |
| | | VQFYGLSENDEWTQDRAKPVTQIVSAEAWGR |
| | | AD |

We claim:

1. A TCR construct comprising a TCR alpha chain polypeptide and a TCR beta chain polypeptide, the TCR alpha chain polypeptide comprising a variable alpha (Vα) domain and a constant alpha (Cα) domain and the TCR beta chain polypeptide comprising a variable beta (Vβ) domain and a constant beta (Cβ) domain,
wherein the Cα domain and Cβ domain comprise stabilizing mutations, the stabilizing mutations comprising a first interchain disulfide bond between the Cα domain and the Cβ domain and one or more additional stabilizing mutations, the one or more additional stabilizing mutations selected from:
a) an interchain disulfide bond formed between: i) a cysteine residue comprised by an amino acid extension at the C-terminus of the Cβ domain of the TCR beta chain polypeptide, wherein the amino acid extension is 1 to about 10 amino acids in length, and ii) a cysteine residue at position TRAC 128 in the Cα domain of the TCR alpha chain polypeptide;
b) an interchain disulfide bond between cysteine residue substitutions at positions TRAC 84.2 and TRBC 79;
c) an interchain disulfide bond between cysteine residue substitutions at positions TRAC 122 and TRBC 12;
d) an interchain disulfide bond between cysteine residue substitutions at positions TRAC 124 and TRBC 11;
e) an interchain disulfide bond between cysteine residue substitutions at positions TRAC 125 and TRBC 11;
f) an interchain disulfide bond between cysteine residue substitutions at positions TRAC 126 and TRBC 11;
g) an interchain disulfide bond between cysteine residue substitutions at positions TRAC 127 and TRBC 11;
h) an interchain disulfide bond between cysteine residue substitutions at positions TRAC 128 and TRBC 11;
i) an intrachain disulfide bond between cysteine residue substitutions at positions TRAC 39 and TRAC 85;
j) an intrachain disulfide bond between cysteine residue substitutions at positions TRAC 26 and TRAC 85.1;
k) an amino acid substitution at position TRAC 4 from Val to Ala, Thr, Ile, Leu or Met;
l) an amino acid substitution at position TRAC 26 from Thr to Ala, Val, Ile, Leu or Met;
m) an amino acid substitution at position TRAC 39 from Val to Ala, Thr, Ile, Leu or Met;
n) an amino acid substitution at position TRAC 85 from Ala to Ser, Thr, Val, Ile or Met;
o) an amino acid substitution at position TRAC 105 from Ala to Ser, Thr, Glu, Gln, Asp, Asn, His, Lys or Arg;
p) an amino acid substitution at position TRAC 120 from Phe to Tyr or His;
q) an amino acid substitution at position TRBC 6 from Val to Ala, Thr, Ile, Leu or Met;
r) an amino acid substitution at position TRBC 36 from His to Phe, Tyr or Trp;
s) an amino acid substitution at position TRBC 86 from Ser to Ala or Thr;
t) an amino acid substitution at position TRBC 45.3 from Val to Ser, Thr, Glu, Gln, Asp, Asn, His, Lys or Arg;
u) a deletion of 1 to 4 consecutive amino acids of the DE loop in the Cβ domain of the TCR construct, and
v) a replacement of the amino acids at positions TRBC 84.4 to 85.4 with an amino acid sequence of 2 to 4 amino acids, wherein the amino acid sequence allows for formation of a beta-turn,
wherein the numbering of amino acids is IMGT numbering,
and wherein the TCR construct has an increased TCR melting temperature (Tm) as compared to a corresponding TCR construct comprising the first non-naturally occurring disulfide bond alone.

2. The TCR construct according to claim 1, wherein the first interchain disulfide bond is selected from:
a) an interchain disulfide bond formed between: i) a cysteine residue comprised by an amino acid extension at the C-terminus of the Cβ domain of the TCR beta chain polypeptide, wherein the amino acid extension is 1 to about 10 amino acids in length, and ii) a cysteine residue at position TRAC 128 in the Cα domain of the TCR alpha chain polypeptide;
b) an interchain disulfide bond between cysteine residue substitutions at positions TRAC 84 and TRBC 79;
c) an interchain disulfide bond between cysteine residue substitutions at positions TRAC 84.2 and TRBC 79;
d) an interchain disulfide bond between cysteine residue substitutions at positions TRAC 122 and TRBC 12;
e) an interchain disulfide bond between cysteine residue substitutions at positions TRAC 124 and TRBC 11;
f) an interchain disulfide bond between cysteine residue substitutions at positions TRAC 125 and TRBC 11;
g) an interchain disulfide bond between cysteine residue substitutions at positions TRAC 126 and TRBC 11;
h) an interchain disulfide bond between cysteine residue substitutions at positions TRAC 127 and TRBC 11, and
i) an interchain disulfide bond between cysteine residue substitutions at positions TRAC 128 and TRBC 11,
and wherein the first interchain disulfide bond and any additional interchain disulfide bonds are different.

3. The TCR construct according to claim 1, wherein the first interchain disulfide bond is selected from:
a) an interchain disulfide bond formed between: i) a cysteine residue comprised by an amino acid extension at the C-terminus of the Cβ domain of the TCR beta chain polypeptide, where the amino acid extension is 1 to about 10 amino acids in length, and ii) a cysteine residue at position TRAC 128 in the Cα domain of the TCR alpha chain polypeptide;
b) an interchain disulfide bond between cysteine residue substitutions at positions TRAC 84 and TRBC 79, and
c) an interchain disulfide bond between cysteine residue substitutions at positions TRAC 84.2 and TRBC 79,
and wherein the first interchain disulfide bond and any additional interchain disulfide bonds are different.

4. The TCR construct according to claim 1, wherein the first interchain disulfide bond is selected from:
a) an interchain disulfide bond formed between: i) a cysteine residue comprised by an amino acid extension at the C-terminus of the Cβ domain of the TCR beta chain polypeptide, where the amino acid extension is 1 to about 10 amino acids in length, and ii) a cysteine residue at position TRAC 128 in the Cα domain of the TCR alpha chain polypeptide, and
b) an interchain disulfide bond between cysteine residue substitutions at positions TRAC 84.2 and TRBC 79,
and wherein the first interchain disulfide bond and any additional interchain disulfide bonds are different.

5. The TCR construct according to any one of claims 1 to 4, wherein the one or more additional stabilizing mutations comprise an additional interchain disulfide bond selected from:
a) an interchain disulfide bond formed between: i) a cysteine residue comprised by an amino acid extension at the C-terminus of the Cβ domain of the TCR beta chain polypeptide, where the amino acid extension is 1 to about 10 amino acids in length, and ii) a cysteine residue at position TRAC 128 in the Cα domain of the TCR alpha chain polypeptide;
b) an interchain disulfide bond between cysteine residue substitutions at positions TRAC 84.2 and TRBC 79;
c) a disulfide bond between cysteine residue substitutions at positions TRAC 122 and TRBC 12;
d) a disulfide bond between cysteine residue substitutions at positions TRAC 124 and TRBC 11;
e) a disulfide bond between cysteine residue substitutions at positions TRAC 125 and TRBC 11;
f) a disulfide bond between cysteine residue substitutions at positions TRAC 126 and TRBC 11;
g) a disulfide bond between cysteine residue substitutions at positions TRAC 127 and TRBC 11, and
h) a disulfide bond between cysteine residue substitutions at positions TRAC 128 and TRBC 11.

6. The TCR construct according to claim 1, wherein the first non-naturally occurring interchain disulfide bond is a disulfide bond between cysteine residue substitutions at positions TRAC 84.2 and TRBC 79.

7. The TCR construct according to claim 6, wherein the one or more additional stabilizing mutations comprise an additional interchain disulfide bond selected from:
a) an interchain disulfide bond formed between: i) a cysteine residue comprised by an amino acid extension at the C-terminus of the Cβ domain of the TCR beta chain polypeptide, where the amino acid extension is 1 to about 10 amino acids in length, and ii) a cysteine residue at position TRAC 128 in the Cα domain of the TCR alpha chain polypeptide;
b) a disulfide bond between cysteine residue substitutions at positions TRAC 122 and TRBC 12;
c) a disulfide bond between cysteine residue substitutions at positions TRAC 124 and TRBC 11;
d) a disulfide bond between cysteine residue substitutions at positions TRAC 125 and TRBC 11;
e) a disulfide bond between cysteine residue substitutions at positions TRAC 126 and TRBC 11;
f) a disulfide bond between cysteine residue substitutions at positions TRAC 127 and TRBC 11, and
g) a disulfide bond between cysteine residue substitutions at positions TRAC 128 and TRBC 11.

8. The TCR construct according to any one of claims 1 to 7, wherein the amino acid extension at the C-terminus of the Cβ domain of the TCR beta chain polypeptide is 5 amino acids or less in length.

9. The TCR construct according to any one of claims 1 to 8, wherein the amino acid extension at the C-terminus of the Cβ domain of the TCR beta chain polypeptide comprises all or a part of a sequence of an IgG1 upper hinge region.

10. The TCR construct according to claim 9, wherein the amino acid extension comprises the sequence:
[SEQ ID NO: 19]
EPKSC.

11. The TCR construct according to claim 9, wherein the amino acid extension comprises the sequence:
[SEQ ID NO: 16]
EPKSCDKTHT.

12. The TCR construct according to any one of claims 1 to 11, wherein the one or more additional stabilizing mutations comprise an intrachain disulfide bond selected from:
an intrachain disulfide bond between cysteine residue substitutions at positions TRAC 39 and TRAC 85, and
an intrachain disulfide bond between cysteine residue substitutions at positions TRAC 26 and TRAC 85.1.

13. The TCR construct according to any one of claims 1 to 12, wherein the one or more additional stabilizing mutations comprise:
a) an amino acid substitution at position TRAC 26 from Thr to Ala, Val, Ile, Leu or Met;
b) an amino acid substitution at position TRAC 85 from Ala to Ser, Thr, Val, Ile or Met;
c) an amino acid substitution at position TRBC 6 from Val to Ala, Thr, Ile, Leu or Met, and
d) an amino acid substitution at position TRBC 36 from His to Phe, Tyr or Trp.

14. A TCR construct comprising a TCR alpha chain polypeptide and a TCR beta chain polypeptide, the TCR alpha chain polypeptide comprising a variable alpha (Vα) domain and a constant alpha (Cα) domain and the TCR beta chain polypeptide comprising a variable beta (Vβ) domain and a constant beta (Cβ) domain,
wherein the Cα domain and/or Cβ domain comprise one or more stabilizing mutations selected from:
a) an interchain disulfide bond formed between: i) a cysteine residue comprised by an amino acid extension at the C-terminus of the Cβ domain of the TCR beta chain polypeptide, wherein the amino acid extension is 1 to about 10 amino acids in length, and ii) a cysteine residue at position TRAC 128 in the Cα domain of the TCR alpha chain polypeptide;
b) an interchain disulfide bond between cysteine residue substitutions at positions TRAC 84.2 and TRBC 79;
c) an interchain disulfide bond between cysteine residue substitutions at positions TRAC 124 and TRBC 11;
d) an interchain disulfide bond between cysteine residue substitutions at positions TRAC 125 and TRBC 11;
e) an interchain disulfide bond between cysteine residue substitutions at positions TRAC 126 and TRBC 11;
f) an interchain disulfide bond between cysteine residue substitutions at positions TRAC 127 and TRBC 11;
g) an interchain disulfide bond between cysteine residue substitutions at positions TRAC 128 and TRBC 11;
h) an intrachain disulfide bond between cysteine residue substitutions at positions TRAC 39 and TRAC 85;
i) an intrachain disulfide bond between cysteine residue substitutions at positions TRAC 26 and TRAC 85.1;
j) an amino acid substitution at position TRAC 4 from Val to Ala, Thr, Ile, Leu or Met;
k) an amino acid substitution at position TRAC 26 from Thr to Ala, Val, Ile, Leu or Met;
l) an amino acid substitution at position TRAC 39 from Val to Ala, Thr, Ile, Leu or Met;
m) an amino acid substitution at position TRAC 85 from Ala to Ser, Thr, Val, Ile or Met;
n) an amino acid substitution at position TRAC 105 from Ala to Ser, Thr, Glu, Gln, Asp, Asn, His, Lys or Arg;
o) an amino acid substitution at position TRAC 120 from Phe to Tyr or His;
p) an amino acid substitution at position TRBC 6 from Val to Ala, Thr, Ile, Leu or Met;
q) an amino acid substitution at position TRBC 36 from His to Phe, Tyr or Trp;
r) an amino acid substitution at position TRBC 86 from Ser to Ala or Thr;
s) an amino acid substitution at position TRBC 45.3 from Val to Ser, Thr, Glu, Gln, Asp, Asn, His, Lys or Arg;
t) a deletion of 1 to 4 consecutive amino acids of the DE loop in the Cβ domain of the TCR construct, and
u) a replacement of the amino acids at positions TRBC 84.4 to 85.4 with an amino acid sequence of 2 to 4 amino acids, wherein the amino acid sequence allows for formation of a beta-turn,
wherein the numbering of amino acids is IMGT numbering,
and wherein the TCR construct has an increased TCR melting temperature (Tm) as compared to a corresponding TCR construct that does not comprise the one or more stabilizing mutations.

15. The TCR construct according to claim 14, further comprising an interchain disulfide bond selected from:
an interchain disulfide bond between cysteine residue substitutions at positions TRAC 84 and TRBC 79, and
an interchain disulfide bond between cysteine residue substitutions at positions TRAC 122 and TRBC 12.

16. The TCR construct according to claim 14 or 15, wherein the stabilizing mutations comprise a first interchain disulfide bond selected from:
a) an interchain disulfide bond formed between: i) a cysteine residue comprised by an amino acid extension at the C-terminus of the Cβ domain of the TCR beta chain polypeptide, wherein the amino acid extension is 1 to about 10 amino acids in length, and ii) a cysteine residue at position TRAC 128 in the Cα domain of the TCR alpha chain polypeptide;
b) an interchain disulfide bond between cysteine residue substitutions at positions TRAC 84.2 and TRBC 79;
c) an interchain disulfide bond between cysteine residue substitutions at positions TRAC 124 and TRBC 11;
d) an interchain disulfide bond between cysteine residue substitutions at positions TRAC 125 and TRBC 11;
e) an interchain disulfide bond between cysteine residue substitutions at positions TRAC 126 and TRBC 11;
f) an interchain disulfide bond between cysteine residue substitutions at positions TRAC 127 and TRBC 11, and
g) an interchain disulfide bond between cysteine residue substitutions at positions TRAC 128 and TRBC 11.

17. The TCR construct according to claim 16, wherein the stabilizing mutations comprise a second interchain disulfide bond selected from:
a) an interchain disulfide bond formed between: i) a cysteine residue comprised by an amino acid extension at the C-terminus of the Cβ domain of the TCR beta chain polypeptide, wherein the amino acid extension is 1 to about 10 amino acids in length, and ii) a cysteine residue at position TRAC 128 in the Cα domain of the TCR alpha chain polypeptide;
b) an interchain disulfide bond between cysteine residue substitutions at positions TRAC 84.2 and TRBC 79;
c) an interchain disulfide bond between cysteine residue substitutions at positions TRAC 124 and TRBC 11;
d) an interchain disulfide bond between cysteine residue substitutions at positions TRAC 125 and TRBC 11;
e) an interchain disulfide bond between cysteine residue substitutions at positions TRAC 126 and TRBC 11;
f) an interchain disulfide bond between cysteine residue substitutions at positions TRAC 127 and TRBC 11, and
g) an interchain disulfide bond between cysteine residue substitutions at positions TRAC 128 and TRBC 11,
wherein the first and second interchain disulfide bonds are different.

18. The TCR construct according to any one of claims 14 to 17, wherein the amino acid extension at the C-terminus of the Cβ domain of the TCR beta chain polypeptide is 5 amino acids or less in length.

19. The TCR construct according to any one of claims 14 to 18, wherein the amino acid extension at the C-terminus of the Cβ domain of the TCR beta chain polypeptide comprises all or a part of a sequence of an IgG1 upper hinge region.

20. The TCR construct according to claim 19, wherein the amino acid extension comprises the sequence:
[SEQ ID NO: 19]
EPKSC.

21. The TCR construct according to claim 19, wherein the amino acid extension comprises the sequence:
[SEQ ID NO: 16]
EPKSCDKTHT.

22. A TCR construct comprising a TCR alpha chain polypeptide and a TCR beta chain polypeptide, the TCR alpha chain polypeptide comprising a variable alpha (Vα) domain and a constant alpha (Cα) domain and the TCR beta chain polypeptide comprising a variable beta (Vβ) domain and a constant beta (Cβ) domain,
wherein the Cα domain and Cβ domain together comprise two or more stabilizing mutations selected from:
a) an interchain disulfide bond formed between: i) a cysteine residue comprised by an amino acid extension at the C-terminus of the Cβ domain of the TCR beta chain polypeptide, wherein the amino acid extension is 1 to about 10 amino acids in length, and ii) a cysteine residue at position TRAC 128 in the Cα domain of the TCR alpha chain polypeptide;
b) an interchain disulfide bond between cysteine residue substitutions at positions TRAC 84 and TRBC 79;
c) an interchain disulfide bond between cysteine residue substitutions at positions TRAC 84.2 and TRBC 79;
d) an interchain disulfide bond between cysteine residue substitutions at positions TRAC 124 and TRBC 11;
e) an interchain disulfide bond between cysteine residue substitutions at positions TRAC 122 and TRBC 12;
f) an interchain disulfide bond between cysteine residue substitutions at positions TRAC 125 and TRBC 11;
g) an interchain disulfide bond between cysteine residue substitutions at positions TRAC 126 and TRBC 11;
h) an interchain disulfide bond between cysteine residue substitutions at positions TRAC 127 and TRBC 11;
i) an interchain disulfide bond between cysteine residue substitutions at positions TRAC 128 and TRBC 11;
j) an intrachain disulfide bond between cysteine residue substitutions at positions TRAC 39 and TRAC 85;
k) an intrachain disulfide bond between cysteine residue substitutions at positions TRAC 26 and TRAC 85.1;
l) an amino acid substitution at position TRAC 4 from Val to Ala, Thr, Ile, Leu or Met;
m) an amino acid substitution at position TRAC 26 from Thr to Ala, Val, Ile, Leu or Met;
n) an amino acid substitution at position TRAC 39 from Val to Ala, Thr, Ile, Leu or Met;
o) an amino acid substitution at position TRAC 85 from Ala to Ser, Thr, Val, Ile or Met;
p) an amino acid substitution at position TRAC 105 from Ala to Ser, Thr, Glu, Gln, Asp, Asn, His, Lys or Arg;
q) an amino acid substitution at position TRAC 120 from Phe to Tyr or His;
r) an amino acid substitution at position TRBC 6 from Val to Ala, Thr, Ile, Leu or Met;
s) an amino acid substitution at position TRBC 36 from His to Phe, Tyr or Trp;
t) an amino acid substitution at position TRBC 86 from Ser to Ala or Thr;
u) an amino acid substitution at position TRBC 45.3 from Val to Ser, Thr, Glu, Gln, Asp, Asn, His, Lys or Arg;
v) a deletion of 1 to 4 consecutive amino acids of the DE loop in the Cβ domain of the TCR construct, and
w) a replacement of the amino acids at positions TRBC 84.4 to 85.4 with an amino acid sequence of 2 to 4 amino acids, wherein the amino acid sequence allows for formation of a beta-turn,
wherein the numbering of amino acids is IMGT numbering,
and wherein the TCR construct has an increased TCR melting temperature (Tm) as compared to a corresponding TCR construct that does not comprise the two or more stabilizing mutations.

23. The TCR construct according to claim 22, wherein the stabilizing mutations comprise a first interchain disulfide bond and a second interchain disulfide bond, wherein the first and second interchain disulfide bonds are different.

24. The TCR construct according to claim 22 or 23, wherein the amino acid extension at the C-terminus of the Cβ domain of the TCR beta chain polypeptide is 5 amino acids or less in length.

25. The TCR construct according to any one of claims 22 to 24, wherein the amino acid extension at the C-terminus of the Cβ domain of the TCR beta chain polypeptide comprises all or a part of a sequence of an IgG1 upper hinge region.

26. The TCR construct according to claim 25, wherein the amino acid extension comprises the sequence:
[SEQ ID NO: 19]
EPKSC.

27. The TCR construct according to claim 25, wherein the amino acid extension comprises the sequence:
[SEQ ID NO: 16]
EPKSCDKTHT.

28. A TCR construct comprising a TCR alpha chain polypeptide and a TCR beta chain polypeptide, the TCR alpha chain polypeptide comprising a variable alpha (Vα) domain and a constant alpha (Cα) domain and the TCR beta chain polypeptide comprising a variable beta (Vβ) domain and a constant beta (Cβ) domain, the TCR construct comprising a combination of amino acid mutations selected from the combinations of amino acid mutations set forth for any one of the variants shown in Table 2, wherein the numbering of amino acids is IMGT numbering.

29. A TCR construct comprising a TCR alpha chain polypeptide and a TCR beta chain polypeptide, the TCR alpha chain polypeptide comprising a variable alpha (Vα) domain and a constant alpha (Cα) domain and the TCR beta chain polypeptide comprising a variable beta (Vβ) domain and a constant beta (Cβ) domain, the TCR construct comprising a combination of amino acid mutations selected from the combinations of amino acid mutations set forth for any one of the variants shown in Table 3, wherein the numbering of amino acids is IMGT numbering.

30. The TCR construct according to any one of claims 1 to 29, wherein the TCR alpha chain polypeptide or the TCR beta chain polypeptide is fused to an immunoglobulin (Ig) Fc region.

31. The TCR construct according to claim 30, wherein the Ig Fc region is an IgG Fc region.

32. The TCR construct according to claim 31, wherein the IgG Fc region is an IgG1 Fc region.

33. The TCR construct according to any one of claims 30 to 32, wherein the TCR beta chain polypeptide is fused to the Ig Fc region.

34. A TCR fusion protein comprising one or more TCR constructs according to any one of claims 1 to 29 and a scaffold, wherein at least one of the TCR constructs is fused to the scaffold.

35. The TCR fusion protein according to claim 34, wherein the scaffold comprises an immunoglobulin (Ig) Fc region.

36. The TCR fusion protein according to claim 35, wherein the Ig Fc region is an IgG Fc region.

37. The TCR fusion protein according to claim 36, wherein the IgG Fc region is an IgG1 Fc region.

38. The TCR fusion protein according to any one of claims 34 to 37, wherein the TCR fusion protein comprises one, two, three or four TCR constructs.

39. The TCR fusion protein according to any one of claims 34 to 37, wherein the TCR fusion protein comprises two or more TCR constructs.

40. The TCR fusion protein according to claim 39, wherein two of the TCR constructs are fused in tandem.

41. The TCR fusion protein according to any one of claims 34 to 40, wherein at least one of the TCR constructs is fused to the scaffold via the TCR beta chain polypeptide.

42. The TCR fusion protein according to any one of claims 34 to 41, wherein the TCR fusion protein further comprises one or more additional biologically active moieties.

43. The TCR fusion protein according to claim 42, wherein the one or more additional biologically active moieties comprise an antigen-binding domain.

44. The TCR fusion protein according to claim 43, wherein the antigen-binding domain is an scFv or a Fab.

45. The TCR fusion protein according to any one of claims 42 to 44, wherein at least one of the biologically active moieties is fused to the scaffold.

46. A pharmaceutical composition comprising a TCR construct according to any one of claims 1 to 33 and a pharmaceutically acceptable carrier or diluent.

47. A pharmaceutical composition comprising a TCR fusion protein according to any one of claims 34 to 45 and a pharmaceutically acceptable carrier or diluent.

48. A polynucleotide or set of polynucleotides encoding a TCR construct according to any one of claims 1 to 33.

49. A polynucleotide or set of polynucleotides encoding a TCR fusion protein according to any one of claims 34 to 45.

50. A method of preparing a TCR construct according to any one of claims 1 to 33 comprising transfecting a cell with a polynucleotide or set of polynucleotides according to claim 48, and culturing the cell under conditions suitable for expression of the TCR construct.

51. A method of preparing a TCR fusion protein according to any one of claims 34 to 45 comprising transfecting a cell with a polynucleotide or set of polynucleotides according to claim 49, and culturing the cell under conditions suitable for expression of the TCR construct.

52. The method according to claim 50 or 51, wherein the cell is cultured at a temperature of 30° C. to 35° C.

53. A method of treating a disease or disorder in a subject in need thereof, the method comprising administering to the subject an effective amount of a TCR construct according to any one of claims 1 to 33, or a TCR fusion protein according to any one of claims 34 to 45.

54. A TCR construct according to any one of claims 1 to 33 for use in therapy.

55. A TCR fusion protein according to any one of claims 34 to 45 for use in therapy.


SEQUENCE LISTING (43 sequences)

SEQ ID NO: 1 - protein, 94 aa, Artificial Sequence
  Human TCR alpha chain constant region (TRAC)
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 2 - protein, 130 aa, Artificial Sequence
  Human TCR beta chain constant region (TRBC1)
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 3 - protein, 130 aa, Artificial Sequence
  Human TCR beta chain constant region (TRBC2)
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 4 - protein, 95 aa, Artificial Sequence
  Human TCR alpha chain constant region (TRAC) including the cysteine residue at position TRAC/128
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSC

SEQ ID NO: 5 - protein, 15 aa, Artificial Sequence
  TCRalpha hinge sequence
ESSCDVKLVEKSFET

SEQ ID NO: 6 - protein, 6 aa, Artificial Sequence
  TCRbeta hinge sequence
DCGFTS

SEQ ID NO: 7 - protein, 16 aa, Artificial Sequence
  TCRgamma hinge sequence
DVITMDPKDNCSKDAN

SEQ ID NO: 8 - protein, 22 aa, Artificial Sequence
  TCRdelta hinge sequence
DHVKPKETENTKQPSKSCHKPK

SEQ ID NO: 9 - protein, 15 aa, Artificial Sequence
  IgG1 hinge sequence
EPKSCDKTHTCPPCP

SEQ ID NO: 10 - protein, 12 aa, Artificial Sequence
  IgG2 hinge sequence
ERKCCVECPPCP

SEQ ID NO: 11 - protein, 17 aa, Artificial Sequence
  IgG3-H1 hinge sequence
ELKTPLGDTTHTCPRCP

SEQ ID NO: 12 - protein, 15 aa, Artificial Sequence
  IgG3-H2, -H3 & -H4 hinge sequence
EPKSCDTPPPCPRCP

SEQ ID NO: 13 - protein, 12 aa, Artificial Sequence
  IgG4 hinge sequence
ESKYGPPCPSCP

SEQ ID NO: 14 - protein, 6 aa, Artificial Sequence
  IgA2 hinge sequence
VPPPPP

SEQ ID NO: 15 - protein, 22 aa, Artificial Sequence
  IgG1 full hinge sequence
EPKSCDKTHTCPPCPAPELLGG

SEQ ID NO: 16 - protein, 10 aa, Artificial Sequence
  IgG1 upper hinge sequence
EPKSCDKTHT

SEQ ID NO: 17 - protein, 5 aa, Artificial Sequence
  IgG1 core hinge sequence
CPPCP

SEQ ID NO: 18 - protein, 7 aa, Artificial Sequence
  IgG1 lower hinge sequence
APELLGG

SEQ ID NO: 19 - protein, 5 aa, Artificial Sequence
  IgG1 partial hinge sequence
EPKSC

SEQ ID NO: 20 - protein, 217 aa, Artificial Sequence
  Human IgG1 Fc sequence 231-447 (EU-numbering)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 15 aa, Artificial Sequence
  IgG1 partial hinge sequence
EPKSCDKTHTCPPCP

SEQ ID NO: 22 - protein, 23 aa, Artificial Sequence
  Signal sequence
MRPTWAWWLFLVLLLALWAPARG

SEQ ID NO: 23 - protein, 9 aa, Artificial Sequence
  NY-ESO1 peptide
SLLMWITQC

SEQ ID NO: 24 - protein, 9 aa, Artificial Sequence
  gp100 peptide
YLEPGPVTA

SEQ ID NO: 25 - protein, 7 aa, Artificial Sequence
  anti-gp100 CDR2a
IRSNERE

SEQ ID NO: 26 - protein, 9 aa, Artificial Sequence
  anti-gp100 CDR3a
ATDGSTPMQ

SEQ ID NO: 27 - protein, 6 aa, Artificial Sequence
  anti-gp100 CDR2b
SWAQGD

SEQ ID NO: 28 - protein, 8 aa, Artificial Sequence
  anti-gp100 CDR3b
ASSWGAPY

SEQ ID NO: 29 - protein, 9 aa, Artificial Sequence
  anti-gp100 CDR3a
ATDGDTPLV

SEQ ID NO: 30 - protein, 8 aa, Artificial Sequence
  anti-gp100 CDR3b
ASSHGAPY

SEQ ID NO: 31 - protein, 6 aa, Artificial Sequence
  anti-gp100 CDR2b
SWGTGD

SEQ ID NO: 32 - protein, 6 aa, Artificial Sequence
  anti-gp100 CDR2b
SWAVGN

SEQ ID NO: 33 - protein, 8 aa, Artificial Sequence
  anti-gp100 CDR3b
ASSIGGPY

SEQ ID NO: 34 - protein, 7 aa, Artificial Sequence
  anti-NY-ESO1 CDR2a
ITPWQRE

SEQ ID NO: 35 - protein, 13 aa, Artificial Sequence
  anti-NY-ESO1 CDR3a
AVRPLLDGTYIPT

SEQ ID NO: 36 - protein, 6 aa, Artificial Sequence
  anti-NY-ESO1 CDR2b
SVAIQT

SEQ ID NO: 37 - protein, 12 aa, Artificial Sequence
  anti-NY-ESO1 CDR3b
ASSYLGNTGELF

SEQ ID NO: 38 - protein, 7 aa, Artificial Sequence
  anti-NY-ESO1 CDR2a
IQSSQRE

SEQ ID NO: 39 - protein, 13 aa, Artificial Sequence
  anti-NY-ESO1 CDR3a
AVRHTSNGYFPPT

SEQ ID NO: 40 - protein, 6 aa, Artificial Sequence
  anti-NY-ESO1 CDR2b
SVGAGT

SEQ ID NO: 41 - protein, 7 aa, Artificial Sequence
  anti-NY-ESO1 CDR2a
ISPWQRE

SEQ ID NO: 42 - protein, 12 aa, Artificial Sequence
  anti-NY-ESO1 CDR3b
ASSYVGDTGELF

SEQ ID NO: 43 - protein, 130 aa, Artificial Sequence
  Human TCR beta chain constant region (TRBC1) including Cys -> Ala mutation at position 85.1
EDLNKVFPPEVAVFEPSEAEISHTQKATLVCLATGFFPDHVELSWWVNGKEVHSGVSTDPQPLKEQPALNDSRYALSSRLRVSATFWQNPRNHFRCQVQFYGLSENDEWTQDRAKPVTQIVSAEAWGRAD